(19) United States
(12) Patent Application Publication
(10) Pub. No.: US 2013/0095071 A1
(43) Pub. Date: Apr. 18, 2013

(54) METHOD OF TREATING OR PREVENTING HEARING LOSS

(71) Applicants: Manohar BANCE, Halifax (CA); George ROBERTSON, Halifax (CA); Jian WANG, Halifax (CA)

(72) Inventors: Manohar BANCE, Halifax (CA); George ROBERTSON, Halifax (CA); Jian WANG, Halifax (CA)

(73) Assignee: Audigen Inc., Halifax (CA)

(21) Appl. No.: 13/528,053

(22) Filed: Jun. 20, 2012

Related U.S. Application Data
(63) Continuation of application No. PCT/CA2010/002037, filed on Dec. 21, 2010.
(60) Provisional application No. 61/288,526, filed on Dec. 21, 2009.

(51) Int. Cl. A61K 47/42

(52) U.S. Cl. CPC A61K 47/42
USPC 424/93.2

(57) ABSTRACT

Disclosed herein is a method of delivering a mutated tyrosine adeno-associated viral vector or a pharmaceutically active agent to an inner ear. The method comprises contacting the round window membrane with the vector or the pharmaceutically active agent, in which the permeability of the round window membrane having been enhanced to allow transport of the vector or the pharmaceutically active agent across it so as to deliver the vector or the pharmaceutically active agent to the inner ear. Also disclosed are methods to prevent or treat hearing loss and impaired balance in human subjects using the delivery method.

### FIELD OF THE INVENTION

The present relates to a method of treating or preventing hearing loss using gene therapy.

### BACKGROUND

Age-related hearing loss (AHL), or presbycusis, is a common neurodegenerative disorder in aged adults, which affects approximately 40% of the population by the age of 65 (NIDCD, 1995). The process of aging interacts with many other factors, such as noise exposure and miscellaneous ototoxic insults which are hazardous to the receptor hair cells (HCs) and the spiral ganglion neurons (SGNs) in the cochlea. In many cases, it is difficult to distinguish between the effects of aging per se and the effects of other hazardous factors on cell death in the cochlea. Permanent hearing loss resulting from the loss of HCs and SGNs is irreversible because the cells are terminally developed and cannot be replaced by mitosis. Although great efforts have been and continue to be made to regenerate lost HCs and SGNs in mammals, these efforts have been largely unsuccessful.

A large body of evidence implicates apoptosis in aging associated cochlea cell death or damage. During the process of aging, apoptosis can be triggered by many different factors that result in caspase activation (Spicer and Schulte, 2002). Activation of these proteases in the cochlea causes the death of HCs and SGNs (Zheng et al., 1998). Caspase inhibitors such as z-DEVD-fmk and z-LEHD-fmk have been shown to protect cochlea HCs from cisplatin-induced death. Direct caspase inhibitor application in the inner ear also greatly enhances vestibular HC survival after an aminoglycoside treatment (Matsui et al., 2003). Other methods shown to partially prevent ototoxin-induced HC loss include the use of minocycline (Wei et al., 2005), neurotrophins (Zheng et al., 1995; Ernfors et al., 1996; Ding et al., 1999a), calpain inhibitors (Wang et al., 1999) and antioxidant therapy (Garetz et al., 1994; Lautermann et al., 1995; Ohinata et al., 2003). A common feature in these treatments is that they all block apoptosis. Unfortunately, the short duration of action of these treatments limits their utility in the treatment of presbycusis.

Members of the inhibitor of apoptosis proteins (IAP) such as X-linked IAP (XIAP), human-IAP1 (HIAP1) and human-IAP2 (HIAP2) inhibit both apoptosis initiators (e.g., caspase-9 by XIAP; caspase-8 by HIAP1 and HIAP2) and apoptosis effectors (caspases-3 and -7 by XIAP) by triggering ubiquitin-mediated degradation of these (Deveraux et al., 1997; Roy et al., 1997; Deveraux et al., 1998; Suzuki et al., 2001). As a result, manipulations that increase IAP expression increase the survival of multiple cell types in response to a variety of apoptotic triggers (e.g., (Liston et al., 1996; Robertson et al., 2000)). For example, virally mediated over-expression of XIAP reduces the loss of CA1 hippocampal neurons and preserves spatial navigation memory after transient forebrain ischemia (Xu et al., 1999) and also delays the death of cultured cerebellar granule neurons following potassium withdrawal (Simons et al., 1999). In hepatocytes, over-expression of HIAP2 inhibits the apoptosis induced by various cytokines (Schoemaker et al., 2002). Blocking caspase activity by IAP over-expression has at least two advantages over the use of exogenous inhibitors. Firstly, virally-mediated IAP expression in the inner ear produces prolonged caspase inhibition (Cooper et al., 2006; Chan et al., 2007), much longer than the duration of inhibition produced by exogenous, small molecule inhibitors. Secondly, XIAP also blocks non-caspase mediated cell death, such as that produced by activation of the c-Jun terminal kinase pathway. This makes XIAP the most potent of all known inhibitors of apoptosis (Deveraux and Reed, 1999; Deveraux et al., 1999a; Kaur et al., 2005). Another advantage over small molecule inhibitors of caspases such as zVAD-fmk or DEVD-fmk is that these are not specific for caspases, they also inhibit other cysteinyl proteases such as calpains and cathepsins (Schotte et al., 1999) and therefore, could interfere with the other cellular functions of these proteases.

XIAP is the prototypical IAP characterized by three baculoviral IAP repeats (BIRs) and the ring zinc finger motif. XIAP has been shown to bind and inhibit caspases-3, -7 and -9 (Deveraux et al., 1997; Roy et al., 1997; Deveraux et al., 1998; Takahashi et al., 1998; Sanna et al., 2002b; Sanna et al., 2002a). XIAP contains three BIR domains. BIR1 and BIR2 are located towards the N-terminus of XIAP and are sufficient to protect cells from Fas-induced apoptosis by binding to caspase-3 and -7. However, the degree of protection is less than that provided by full length XIAP. BIR3 is close to the RING domain that is located towards the c-terminus of XIAP (Deveraux et al., 1999b). It has been found that BIR3 alone is sufficient to inhibit the apoptotic initiator caspase-9 (Takahashi et al., 1998; Sun et al., 1999). XIAP binding to pro-caspases-3 and -7 prevents these proteins from being activated by caspase-8. XIAP can also directly inhibit activation of caspases-3 and -7, and accelerate the degradation of these proteins (Deveraux and Reed, 1999; Suzuki et al., 2001). In the intrinsic pathway, XIAP prevents activation of the initiator caspase-9 by cytochrome c. Moreover, XIAP blocks the feedback activation of caspases-8 and -9 by activate caspase-3. From this evidence, we can infer that XIAP is able to block or reduce apoptosis occurring through both the extrinsic and extrinsic pathways.

During recent years, XIAP based gene therapy has been evaluated in many different settings. For example, over-expression of XIAP through genetic manipulation has been demonstrated to be sufficient to prevent neuronal death in models of stroke and Parkinson's disease (Xu et al., 1999; Crocker et al., 2003; Trapp et al., 2003). The survival advantage offered by XIAP over-expression is conferred by inhibition of at least two cell death pathways: inhibition of caspase-3 (Deveraux and Reed, 1999) and c-Jun N-terminal kinase (JNK) (Igaki et al., 2002) as well as being critically involved in the reduction of damaging reactive oxygen species (ROS) by the up-regulation antioxidant genes thereby reducing ROS-mediated cell death (Resch et al., 2008) Resch U, Schichl Y M, Sattler S, de Martin R (2008) Biochem Biophys Res Commun. October 10; 375(1):156-61. Epub 2008 Aug. 8.

Gene therapy may therefore be used to prevent the death of HCs and SGNs by delivering anti-apoptotic genes such as XIAP to these sensory cells. The best anatomical approach to these cells is through the round window membrane (RWM) of the cochlea. Viral vectors are the most effective method to achieve gene transfer in the auditory system. Three groups of viral vectors have been tested including lentivirus, adenovirus and adeno-associated virus (AAV). Among these, AAV vectors appear to be the best choice for inner ear gene therapy because they produce long lasting expression of transfected genes (Cooper et al, 2006) Cooper L B, Chan D K, Roediger F C, Shaffer B R, Fraser J F, Musatov S, Selesnick S H, Kaplitt M G. AAV-mediated delivery of the caspase inhibitor XIAP protects against cisplatin ototoxicity. Otol Neurotol. 2006 June; 27(4):484-90. and have the lowest risk of pathogenic reactions (Kaplitt et al., 1994). Kaplitt M G, Leone P, Samulski R J, Xiao X, Pfaff D W, O'Malley K L, During M J. Long-term gene expression and phenotypic correction using adeno-associated virus vectors in the mammalian brain. Nat. Genet. 1994 October; 8(2):148-54. The RWM is not permeable to AAV requiring the development of new methodologies to safely (no risk of hearing loss) increase the permeability of the RWM to these vectors.

However, despite the aforesaid advances, the effectiveness of XIAP for the treatment of hearing loss remains to be proved and the appropriate methods for this application remain to be explored.

### SUMMARY OF THE INVENTION

We have discovered a novel method to enhance the permeability of the round window membrane (RWM). Our method now allows mutated tyrosine adeno-associated viral expression vectors or pharmaceutically active agents to be transported across the membrane to deliver the vector or the agent into the inner ear. Our method addresses a significant unmet medical need and provides surgeons with a minimally invasive procedure by which to deliver viral expression vectors or pharmaceutically active agents to the inner ear so as to treat or prevent hearing loss or impaired balance in subjects.

Accordingly, in one aspect, there is provided a method of delivering a mutated tyrosine adeno-associated viral vector or a pharmaceutically active agent to an inner ear, the method comprising: contacting the round window membrane with the mutated tyrosine adeno-associated viral vector or the pharmaceutically active agent, the permeability of the round window membrane having been enhanced to allow transport of the mutated tyrosine adeno-associated viral vector or the pharmaceutically active agent across the round window membrane so as to deliver the mutated tyrosine adeno-associated viral vector or the pharmaceutically active agent to the inner ear.

Accordingly in another aspect, there is provided a method of treating or preventing hearing loss in a subject, the method comprising: contacting the round window membrane with a mutated tyrosine adeno-associated viral vector or a pharmaceutically active agent, the permeability of the round window membrane having been enhanced to allow transport of the mutated tyrosine adeno-associated viral vector or the pharmaceutically active agent across the round window membrane so as to deliver the mutated tyrosine adeno-associated viral vector or the pharmaceutically active agent to an inner ear thereby treating or preventing the hearing loss.

Accordingly, in another aspect, there is provided a method of treating hereditary hearing loss in a subject, the method comprising: contacting the round window membrane of the subject with a mutated tyrosine adeno-associated viral expression vector expressing a gene responsible for hereditary hearing loss, the permeability of the round window membrane having been enhanced to allow transport of the vector across the round window membrane, the gene responsible for hereditary hearing loss being positioned in the mutated tyrosine adeno-associated expression vector for expression in an inner ear organ, or associated neural structures, of the subject so as to treat or prevent the hearing loss.

Accordingly, there is provided a method of treating or preventing impaired balance or impaired vestibular function in a subject, the method comprising: contacting the round window membrane of the subject with a mutated tyrosine adeno-associated viral expression vector, the permeability of the round window membrane having been enhanced to allow transport of the mutated tyrosine adeno-associated viral vector across the round window membrane so as to deliver the mutated tyrosine adeno-associated viral expression vector to a cell of the vestibular organ, or associated neural structures, thereby treating or preventing impaired balance or impaired vestibular function.

In yet another aspect, there is provided a method of reducing inner ear cell loss, the method comprising: contacting the inner ear cell with a mutated tyrosine adeno-associated viral vector encoding X-linked inhibitor of apoptosis protein (XIAP), the mutated tyrosine adeno-associated viral vector having been transported across the round window membrane, the permeability of the round window membrane having been enhanced, the XIAP being positioned in the mutated tyrosine adeno-associated viral vector for expression in the inner ear cell, or associated neural structures, so as to reduce the loss of the inner ear cell or associated neural structures.

In yet another aspect, there is provided a method of reducing hair cell loss, the method comprising: contacting the hair cell with a mutated tyrosine adeno-associated viral vector encoding X-linked inhibitor of apoptosis protein (XIAP), the mutated tyrosine adeno-associated viral vector having been transported across the round window membrane, the permeability of the round window membrane having been enhanced, the XIAP being positioned in the mutated tyrosine adeno-associated viral vector for expression in the hair cell, or associated neural structures, so as to reduce loss of the hair cell or associated neural structures.

In another aspect, there is provided a method of reducing spiral ganglion neuron loss, the method comprising: contacting the spiral ganglion neuron with a mutated tyrosine adeno-associated viral vector encoding X-linked inhibitor of apoptosis protein (XIAP), the mutated tyrosine adeno-associated viral vector having been transported across the round window membrane, the permeability of the round window membrane having been enhanced, the XIAP being positioned in the mutated tyrosine adeno-associated viral vector for expression in the spiral ganglion neuron so as to reduce loss thereof.

In one aspect, there is provided a method of treating or preventing degeneration of the vestibular organ or associated neural structures in a subject, the method comprising: contacting a cell of the vestibular organ with a mutated tyrosine adeno-associated viral vector encoding X-linked inhibitor of apoptosis protein (XIAP), the mutated tyrosine adeno-associated viral vector having been transported across the round window membrane, the permeability of the round window membrane having been enhanced, the XIAP being positioned in the mutated tyrosine adeno-associated viral vector for expression in the cell of the vestibular organ or associated neural structures so as to treat or prevent the vestibular organ degeneration.

In still another aspect, there is provided a method of slowing the development of impaired balance in a subject, the method comprising: contacting a cell of the vestibular organ or associated neural structures with a mutated tyrosine adeno-associated viral vector encoding X-linked inhibitor of apoptosis protein (XIAP), the mutated tyrosine adeno-associated viral vector having been transported across the round window membrane, the permeability of the round window membrane having been enhanced, the XIAP being positioned in the mutated tyrosine adeno-associated viral vector for expression in the cell of the vestibular organ or associated neural structures so as to slow the development of the impaired balance.

In one example, the mutated tyrosine adeno-associated viral expression vector expresses an ototoregenerative gene or an ototoprotective gene, the ototoregenerative gene or the ototoprotective gene being positioned in the mutated tyrosine adeno-associated viral vector for expression in an inner ear organ, or associated neural structures.

In another example, the permeability of the round window membrane is enhanced by contacting it with a protease or a biocompatible detergent for a time sufficient to cause the round window membrane to become partially disrupted to permit the mutated tyrosine adeno-associated viral vector or the pharmaceutically active agent to be transported thereacross. The protease partially digests the membrane. The protease is selected from the group consisting of: serine proteases (chymotrypsin, trypsin, elastase), threonine proteases (proteasome hydrolases), cysteine proteases (actinidain, bromelain, calpains, caspases, cathepsins, Mir1-CP, papain), aspartate proteases (cathepsin D, pepsin, chymosin), metalloproteases (collagenase, elastase, gelatinase), and glutamic acid proteases. The biocompatible detergent is selected from the group consisting of: Triton X-100, Triton X-114, NP-40, Brij-35; Brij-58, Tween 20, Tween 80, Octyl glucoside, Octyl thioglucoside, SDS, CHAPS, CHAPSO, Pluronic F-127, and surfactants (Teepol, Lissapol, Alconox).

In another example, the permeability of the round window membrane is enhanced by disruption thereof using electroporation or electropermeabilization.

In another example, the permeability of the round window membrane is enhanced by contacting it with a solution containing an agent that promotes lipid peroxidation for a time sufficient to cause the round window membrane to become partially disrupted.

In another example, the permeability of the round window membrane is enhanced by irrigating the round window membrane with artificial perilymph for a time sufficient to cause the round window membrane to become partially disrupted.

In another example, the permeability of the round window membrane is enhanced by contacting the round window membrane with hyperosmolar or hyposmolar liquids or solids for a time sufficient to cause the round window membrane to become partially disrupted.

In another example, the permeability of the round window membrane is enhanced by passing air over it causing a mild drying effect.

In another example, the mutated tyrosine adeno-associated viral vector is mutated at one or more surface-exposed tyrosine residues on capsid proteins. The mutated tyrosine adeno-associated viral vector is selected from the group consisting of: Tyr252 to Phe272 (Y252F), Tyr272 to Phe272 (Y272F), Tyr444 to Phe444 (Y444F), Tyr500 to Phe500 (Y500F), Tyr700 to Phe700 (Y700F), Tyr704 to Phe704), Tyr730 to Phe730 (Y730F), and Tyr 733 to Phe733 (Y733F). The mutated tyrosine adeno-associated viral vector is Tyr 733 to Phe733 (Y733F).

In one example, the otoprotective gene is an anti-apoptotic gene, a gene encoding anti-oxidant enzymes belonging to the superoxide dismutase (SOD) family, a gene encoding neurotrophic/neuroprotective factors, a gene encoding anti-inflammatory proteins, or a gene that promotes hair cell regeneration in the vestibular system. The otoprotective gene is selected from the group consisting of: Birc1a (NAIP), Birc2 (c-IAP1/HIAP-2), Birc3 (cIAP-2/HIAP-1), Birc4 (XIAP), Birc5 (survivin), Birc6 (apollon), Birc7 (livin), Birc8 (TsIAP); members of the Bcl-2 family: Bcl-2, Bcl-XL, Bcl-w, Mcl-1, Bcl-2L10, BFL-1; endogenous inhibitors of the c-Jun N-terminus kinase (JNK) known as Jun-interacting protein (JIP), JIP-1, JIP-2, JIP-3, JIP-4; SOD1, SOD2; catalase; peroxiredoxin-1, peroxiredoxin-2, glutathione preoxidase 1 (Gpx1), Gpx2, Gpx3, or Gpx4; NGF, BDNF, CNTF, GDNF, Growth/differentiation factor-15 (GDF-15), erythropoietin or vascular endothelial growth factor (VEGF); interleukin-10 (IL-10); glutathione S-transferase, Annexin-1 (ANXA1), or inhibitor of NF-κB (IκB); and ATOH-1. The ototoprotective gene is full length human XIAP. A ubiquitin promoter is used to drive expression of XIAP in cochlea cells.

In one example, the hearing loss is presbycusis.

In another example, the hearing loss is high-frequency hearing loss. The high-frequency hearing loss is at 2 kHz and above.

In yet another example, the hearing loss is due to ototoxicity, noise induced hearing loss, viral infections of the inner ear, autoimmune inner ear diseases, genetic hearing losses, inner ear barotrauma; physical trauma, or surgical trauma; or inflammation. The ototoxicity results from cisplatin treatment of the subject suffering from cancer.

In one example, the inner ear organ includes the inner ear hair cell and the outer ear hair cell. The inner ear cell is a hair cell, a supporting cell, inner ear mechanical structure or a spiral ganglion neuron.

In one example, the gene is selected from the group consisting of: ACTG1, ATP2B2, CDH23, CLDN14, COCH, COL11A2, DFNA5, DFNB31, DFNB59, ESPN, EYA4, GJB2, GJB3, GJB6, KCNQ4, LHFPL5, MT-RNR1, MT-TS1, MYO1A, MYO6, MYO7A, MYO15A, OTOF, PCDH15, POU3F4, SLC26A4, STRC, TECTA, TMC1, TMIE, TMPRSS3, TRIOBP, USH1C and WFS1.

In one example, the hereditary hearing loss is Usher's I syndrome, Usher's II syndrome or Usher's III syndrome.

In one example, the impaired balance is in a subject who is aging.

In another example, the impaired vestibular function is result of vestibular organ degeneration. The vestibular organ regeneration is due to ototoxicity, viral infections of the inner ear, autoimmune inner ear diseases, genetic vestibular losses, inner ear barotraumas; or physical trauma, or surgical trauma.

In one example, the subject is human.

### BRIEF DESCRIPTION OF THE DRAWINGS

In order that the present may be readily understood, embodiments are illustrated by way of example in the accompanying drawings.

FIG. 1 illustrates ABR threshold audiograms in young (2-6 month old) WT and TG mice. A: ABR audiograms for TG and WT mice at months of age; B: ABR audiograms for TG and WT mice at 6 months of age. C: Aging-related hearing loss at 2 and 6 months of age in WT littermates. D: Aging-related hearing loss at 2 and 6 months of age in TG mice. Each circle represents mean±SEM of 15-17 animals. Asterisks in A and B indicate the frequencies at which the differences were statistically significant between the two groups, p<0.05. Hearing status is evaluated with ABR and compared between transgenic (TG) mice in which XIAP is over-expressed and wild type (WT) littermates.

FIG. 2 illustrates ABR threshold audiograms at 10-14 months in WT and TG mice. A: At 10 months hearing thresholds across all frequencies were superior for TG relative to WT mice. B: At 12 months, hearing thresholds for WT and TG mice are similar at low frequencies (2, 4 and 8 kHz). C: At 14 months of age, the averaged ABR thresholds were similar for WT and TG mice at the three low frequencies (2, 4 and 8 kHz) tested while TG mice still displayed superior ABR sensitivities at the higher frequencies. In B and C, asterisks indicate the frequencies at which TG mice displayed superior ABRs (p<0.05). Each circle represents the mean±SEM of 15-17 animals.

FIG. 3 illustrates ABR threshold audiograms showing a comparison of aging-related hearing loss in WT littermates and TG mice. A: The ABR thresholds were averaged into two distinct frequency segments: 2-8 kHz as the low-frequency (LF) region (solid symbols) and 16-64 kHz as the high-frequency (HF) region (open symbols). B: The ABR-threshold audiogram from WT group at 6 months (dashed line, open circle) is compared with that from TG group at 14 months (solid line, solid circle). In A, each point represents the mean of 15-17 animals. In B, each circle represents the mean±SEM of 15-17 animals.

FIG. 4 are graphs showing hair cell loss as a percentage for both TG and WT mice (n=19 per group). Filled and open circles represent the mean±SEM for hair cell loss in TG mice and WT littermates, respectively.

FIG. 5 are representative hair cell loss images from one TG cochlea (Left) and one WT cochlea. The samples were treated with SDH staining. The images were taken from matched spots in the basal turns of the two cochleae. “Basal-1” is about 0.5 mm to the basal end of the cochlea (92% from the apex), while “basal-2” is located 1.2 mm from the basal end.

FIG. 6 is a representative Western blot showing Myc-XIAP and endo-XIAP levels in the ear and brain (temporal lobe) of 2 and 14 month old WT and TG animals. Endo-XIAP levels were higher in the ear than brain, particularly in older age mice at 14 months (14 mo).

FIG. 7 is a histogram showing the impact of all three factors (genotype, tissue and age) on the levels of endo-XIAP. Bars represent mean±SEM. Endogenous-XIAP levels were found to be higher in ears than in brains in both genotypes at 14 months compared to 2 months of age. The difference was statistically significant at 14 months of age. *p<0.05 relative to brain.

FIG. 8 is a histogram showing quantification of Myc-XIAP levels in brain and ears at 2 and 14 months of age. Each bar represents the mean±SEM. At both ages, Myc-XIAP was significantly higher in brain than ear (p<0.05).

FIG. 9 is a TEM image of a cross section from a normal round window membrane. Method of cochlear gene transfection mediated by AAV vector through round window membrane (RWM) is explored. RWM is not permeable to AAV.

FIG. 10 is a photograph showing transfection of inner ear cells seen in surface preparation. GFP positive cells were seen in IHC region, not in the OHC region.

FIG. 11 are SEM images of RWM surface facing middle ear.

FIG. 12 shows the TEM images of damaged RWM at the surface to middle ear, immediately after the treatment of the enzyme. Digesti on of RWM with collagenase makes RWM permeable to AAV.

FIG. 13 is a TEM image of RWM immediately after the digestion showing damage to the epithelia cell.

FIG. 14 are TEM (left) and SEM (right) images from RWM 3 weeks after the digestion treatment, which show no difference from normal control sample. The damage to RWM by the treatment is temporary.

FIG. 15 shows the transfection of a cochlea to which none-tyrosine mutant AAV2 is used with the help of an enzyme. However, the expression of GFP is limited to inner hair cells (IHCs).

FIG. 16 shows the transfection of a cochlea to which AAV8 with tyrosine mutation at 733 is used. Both OHCs and IHCs are transfected. Thus, tyrosine-mutant AAV enhances the transfection of outer hair cells.

FIG. 17: Comparison of threshold shifts between WT and TG mice at 1 and 4 weeks after the noise exposure at 125 dB for 6 hours. All frequency points measured were significantly different between TG and WT mice (p<0.05). The vertical bars represent SEM.

FIG. 18: Mean of IHC and OHC loss (±S.E.M.) measured from the percent distance of the apex along the basilar membrane.

FIG. 19. Representative images of cochlear HC loss from a control animal (left panel) and a transgenic animal (right panel). The upper panels represent the apex of the cochlea and the lowers panels the basal end. Note the greater proportion of missing OHCs and IHCs in the control animal concentrated in the basal region. Scattered HC loss located in the apical region above the lesion in the basal area may be due to the mechanical preparation process.

FIG. 20A and FIG. 20B illustrate a cross-section of a mouse cochlea. A: along the modiolus to show the Rosenthal canal and SGNs; B: sections along the a-a line in A to show the Hebanular perforates. The noise-induced loss of fibers was seen in this cochlea as indicated by the arrow in B.

FIG. 21 illustrates images of the Hebanular perforate in a control cochlea receiving no noise exposure.

FIG. 22 illustrates images of the Hebanular perforate in the noise-damaged cochlea from both the basal turn (upper panel) and apical turn (lower panel).

FIG. 23 illustrates images of SGNs from cochlear cross-sections of the Rosenthal canal from both a normal control cochlea (left) and a noise-damaged cochlea from a WT mouse (right) from the apical (upper panel) and basal turn (lower panel).

FIG. 24 illustrates HC transfection using rAAV-8-GFP with a mutation at tyrosine residue 733. The titer of the vector was 7.5×10¹³. A and B: HC transfection at both basal (A) and 2^{nd} turn (B) via RWM approach. C: transfection of HC at 2″ turn in a cochlea with transfection via cochleostomy.

FIG. 25 illustrates inner (green cells on left side of side of Figure) and outer sensory hair cells (weakly stained cells on the right side of the Figure) of the cochlea that are expressing XIAP-XIAP derived from the AAV8-mut-XIAP-6myc vector that was delivered through the RWM using his enzymatic method as described herein. The AAV8 vector used was the AAV8-733 in which tyrosine 733 is substituted for alanine. The vector strength was 1×10E14 particles/ml used in exactly the same way as before. An antibody against 6myc s used for staining thereby demonstrating that the vector has crossed through the RWM using the methods described herein and infected the inner hair cells and outer hair cells with XIAP.

Further details and advantages will be apparent from the detailed description included below.

### DETAILED DESCRIPTION

In the following description of the embodiments, references to the accompanying drawings are by way of illustration of an example by which the discovery may be practiced. It will be understood that other embodiments may be made without departing from the scope of the discovery disclosed.

Unless otherwise specified, the following definitions apply:

The singular forms “a”, “an” and “the” include corresponding plural references unless the context clearly dictates otherwise.

As used herein, the term “comprising” is intended to mean that the list of elements following the word “comprising” are required or mandatory but that other elements are optional and may or may not be present.

As used herein, the term “consisting of” is intended to mean including and limited to whatever follows the phrase “consisting of”. Thus, the phrase “consisting of” indicates that the listed elements are required or mandatory and that no other elements may be present.

As used herein, the term “subject” or “patient” is intended to mean humans and non-human mammals such as primates, cats, dogs, swine, cattle, sheep, goats, horses, rabbits, rats, mice and the like.

As used herein, the term “mutated tyrosine adeno-associated viral vector” or “mutated AAV” is intended to mean an AAV vector which is mutated at one or more surface-exposed tyrosine residues on capsid proteins. These mutated vectors avoid degradation by the proteasome, and thus significantly increase the transduction efficiency thereof. The vector is an adeno-associated viral expression vector is selected from AAV1, AAV2, AAV3, AAV4, AAV5, AAV6, AAV7 and AAV8. In one example, the adeno-associated viral expression vector is AAV2. In another example, the adeno-associated viral expression vector is a modified serotype-2 or -8 AAV vector. Specific examples of mutated tyrosine adeno-associated viral vector include, for example, but not limited to, mutations of Tyr252 to Phe272 (Y252F), Tyr272 to Phe272 (Y272F), Tyr444 to Phe444 (Y444F), Tyr500 to Phe500 (Y500F), Tyr700 to Phe700 (Y700F), Tyr704 to Phe704), Tyr730 to Phe730 (Y730F), and Tyr 733 to Phe733 (Y733F). In one specific example, the mutated AAV vector is Tyr 733 to Phe733 (Y733F).

As used herein, the term “associated neural structures” when used in conjunction with the inner ear cell associated neural structures, is intended to mean the neural processes, both efferent and afferent that contact or influence the inner ear hair cell function and transmit hair cell activity centrally to the brain, or from the brain to the inner ear.

As used herein, the term “ototoprotective genes” are genes which, when expressed within sensory hair cells and spiral ganglion neurons of the cochlea and vestibular system using tyrosine mutant AAVs, will prevent cell death associated with hearing loss and balance disorders. Examples of ototoprotective genes include anti-apoptotic genes, which include members of the inhibitor of apoptosis family: Birc1a (NAIP), Birc2 (c-IAP1/HIAP-2), Birc3 (cIAP-2/HIAP-1), Birc4 (XIAP), Birc5 (survivin), Birc6 (apollon), Birc7 (livin), Birc8 (TsIAP); members of the Bcl-2 family: Bcl-2, Bcl-XL, Bcl-w, Mcl-1, Bcl-2L10, BFL-1; endogenous inhibitors of the c-Jun N-terminus kinase (JNK) known as Jun-interacting protein (JIP), JIP-1, JIP-2, JIP-3, JIP-4. Also included are genes which encode anti-oxidant enzymes belonging to the superoxide dismutase (SOD) family: SOD1, SOD2; catalase; peroxiredoxin-1, peroxiredoxin-2, glutathione preoxidase 1 (Gpx1), Gpx2, Gpx3, Gpx4. Also included are genes which encode neurotrophic/neuroprotective factors such as NGF, BDNF, CNTF, GDNF, Growth/differentiation factor-15 (GDF-15), erythropoietin and vascular endothelial growth factor (VEGF). Also included are genes which encode anti-inflammatory proteins such as interleukin-10 (IL-10); glutathione S-transferase, Annexin-1 (ANXA1), inhibitor of NF-κB (IκB).

As used herein, the term “ototoregenerative genes” is intended to mean genes that promote hair cell regeneration in the vestibular system such as TGF-Beta or cochlea and includes genes such as ATOH-1.

As used herein, the term “gene responsible for hereditary hearing loss” is a gene that is responsible for hereditary forms of deafness includes the following genes ACTG1, ATP2B2, CDH23, CLDN14, COCH, COL11A2, DFNA5, DFNB31, DFNB59, ESPN, EYA4, GJB2, GJB3, GJB6, KCNQ4, LHFPL5, MT-RNR1, MT-TS1, MYO1A, MYO6, MYO7A, MYO15A, OTOF, PCDH15, POU3F4, SLC26A4, STRC, TECTA, TMC1, TMIE, TMPRSS3, TRIOBP, USH1C or WFS1 (see: http://ghr.nlm.nih.qov/condition=nonsyndromicdeafness)

As used herein, the term “XIAP” is X-linked inhibitor of apoptosis protein and is intended to mean any polypeptide having the activity of full-length human XIAP protein. to This activity is characterized by inhibition of apoptosis and/or binding caspase 3. Examples of XIAP includes full length XIAP, including human XIAP (e.g., genbank accession numbers aac50373, cab95312, aah32729, np.sub.-001158, aaw62257, aac50518, aax29953, Q9R016, aah71665, and cai42584), and XIAP xenologues. Examples of XIAP xenologues are mouse XIAP (e.g., genbank accession numbers q60989 and np.sub.-033818), rat XIAP (e.g., genbank accession numbers aag22969, aag41193, and aag41192), domestic cow (e.g., genbank accession numbers xp.sub.-583068 and np.sub.-001030370), zebrafish (e.g., genbank accession numbers np.sub.-919377, aah55246, and xp.sub.-689837), chimpanzee (e.g., genbank accession number xp.sub.-529138), dog (e.g., genbank accession number abb03778), chicken (e.g., genbank accession number np.sub.-989919), frog (e.g., genbank accession number np.sub.-001025583 and bad98268), orangutan (e.g., genbank accession number cah91479), and catfish (e.g., genbank accession number aax35535).

The term “XIAP” also means any functional XIAP fragment, or any fusion of functional XIAP fragments. Examples of these fragments include those that consist of, consist essentially of, or include (i) BIRs 1-3, (ii) BIR3 and the RZF, (iii) BIR 3 (or a conformationally stabilized BIR of Ts-IAP, TIAP, hILP-2, or birc8), (iv) BIR2-3, (v) BIR2 and the RZF, (vi) BIR1-2, or (vii) BIR2 alone. Furthermore, “XIAP” embraces any of these fragments having an additional amino terminal methionine.

The term “XIAP” also means any fusion of full length XIAP, or a functional fragment thereof, with another polypeptide. These fusions include, but are not limited to, GST-XIAP, HA tagged XIAP, or Flag tagged XIAP. These additional polypeptides may be linked to the N-terminus and/or C-terminus of XIAP.

The term “XIAP” also includes any chimeric XIAP protein. By “chimeric XIAP” is meant a protein comprising a fusion of a XIAP domain or domains with a portion of another protein, wherein the chimeric XIAP retains the properties of human XIAP. Examples of chimeric XIAP proteins include the fusion of any of the above XIAP domains, or fragments thereof, to any domain or fragment of the following proteins such that the family has been termed Baculoviral inhibitor of apoptosis repeat-containing (Birc); Bird (NAIP1); Birc2 (cIAP1); Birc3 (cIAP2); Birc4 (XIAP); Birc5 (Survivin); Birc6 (apollon); Birc7 (livin); Birc8 (TsIAP).

The term “XIAP” is meant to include any protein with at least 70% sequence identity with human XIAP. The term also includes any conservative substitutions of amino-acid residues in XIAP. The term “conservative substitution” refers to replacement of an amino acid residue by a chemically similar residue, e.g., a hydrophobic residue for a separate hydrophobic residue, a charged residue for a separate charged residue, etc. Examples of conserved substitutions for non-polar R groups are alanine, valine, leucine, isoleucine, proline, methionine, phenylalanine, and tryptophan. Examples of substitutions for polar, but uncharged R groups are glycine, serine, threonine, cysteine, asparagine, or glutamine. Examples of substitutions for negatively charged R groups are aspartic acid or glutamic acid. Examples of substitutions for positively charged R groups are lysine, arginine, or histidine. Furthermore, the term XIAP includes conservative substitutions with non-natural amino-acids.

As used herein, the term “pharmaceutically active agent” means a compound that causes a pharmacological effect in a subject. Typically, the pharmacological effect is treating or preventing hearing loss or impaired balance in the subject. The pharmaceutically active agent can include a drug in its biologically active form, a pro-drug in a form such that the biologically active drug form is created in vivo in the subject, a drug metabolite, a pharmaceutically acceptable salt or ester of a biologically active drug, another therapeutically acceptable form of a biologically active drug, or some combination thereof. In the present context, the pharmaceutically active agent may be decadron or other corticosteroids which, when applied to a gelfoam pad, will result in higher perilymph concentrations after they are applied to the round window membrane, the permeability of which has been enhanced using partial enzyme degradation/digestion. Similarly, neuroprotective small molecules such as caspase inhibitors, JNK inhibitors, calpain inhibitors, glutamate receptor antagonists or ion channel blockers, when applied to a gelfoam pad, will also result in higher perilymph concentrations after they are applied to the round window membrane, the permeability of which has been enhanced using partial enzyme degradation/digestion.

As used herein, the term “treating” is intended to mean the administration of a therapeutically effective amount of one of the AAV vectors described herein to a subject who is experiencing loss or impairment of hearing, loss or impairment of balance, or injury to or loss of vestibular hair cells, neurons, supporting cells, or dark cells, in order to minimize, reduce, or completely prevent or restore, the loss of hearing, the loss of balance function or of hair cells, neurons or dark cells of the vestibular portion of the inner ear. Treatment is intended to also include the possibility of inducing, causing or facilitating regeneration of the cellular elements of the inner ear including hair cells, supporting cells, dark cells, neurons and subcellular organelles of these cells including, synapses, stereocilia bundles, kinocilia, mitochondria and other cell organelles, or mechanical and functional supporting structures such as otoconia, cupula and crista of the inner ear. Treatment is also intended to prevent recurrent degeneration after regeneration of cellular elements of the inner ear, including hair cells, supporting cells, dark cells, neurons and subcellular organelles of these cells including synapses, stereocilia bundles, kinocilia, mitochondria and other cell organelles, or mechanical and functional supporting structures such as otoconia, cupula and crista of the inner ear. Treatment is also intended to mean the partial or complete restoration of hearing or balance function regardless of the cellular mechanisms involved.

As used herein, “loss of balance” or “impairment to the sense balance”, “impaired balance”, “loss of balance function” and “balance disorders” are terms that are intended to refer to a deficit in the vestibular system including associated neural structures, or vestibular function of a subject compared to the system of a normally functioning human. This deficit may completely or partially impair a subject's ability to maintain posture, spatial orientation, locomotion and any other functions associated with normal vestibular function. Balance disorders also include intermittent attacks of vertigo, such as those seen in Meniere's Disease, or other inner ear disorders.

As used herein, the term “administration” is intended to include, but is not limited to, the following delivery methods: topical, including topical delivery to the round window membrane of the cochlea, oral, parenteral, subcutaneous, transdermal, and transbuccal administration. In one example, the permeability of the round window membrane is enhanced by partially digesting it using a protease prior to transfection of the inner ear cells with an AAV vector described herein.

As used herein the term “hearing loss” is intended to mean any reduction in a subject's ability to detect sound. Hearing loss is defined as a 10 decibel (dB) standard threshold shift or greater in hearing sensitivity for two of 6 frequencies ranging from 0.5-6.0 (0.5, 1, 2, 3, 4, and 6) kHz (cited in Dobie, R. A. (2005) Audiometric Threshold Shift Definitions: Simulations and Suggestions, Ear and Hearing 26(1) 62-77). Hearing loss can also be only high frequency, and in this case would be defined as 5 dB hearing loss at two adjacent high frequencies (2-6 kHz), or 10 dB at any frequency above 2 kHz. One example of hearing loss is age-related (or aging-related) hearing loss, which is the gradual onset of hearing loss with increasing age.

As used herein, the term “prevention”, in the context of the loss of or impairments to the sense of balance, death or injury of vestibular hair cells, death or injury of vestibular neurons, injury to functionally important mechanical structures such as the ototoconia or cupula, death or injury of vestibular dark cells and the like refers to minimizing, reducing, or completely eliminating the loss or impairment of balance function or damage, death or loss of those cells through the administration of an effective amount of one of the vectors described herein, ideally before an oxidatively stressful insult, or less ideally, shortly thereafter.

Alternatively, the term “prevention” or “preventing” in the context of hearing loss is intended to refer to a significant decrease is the loss of hearing sensitivity within the aforesaid frequency range, particularly at the high frequency range above 3-4 kHz.

### I. Enhancing the Round Window Membrane Permeability

The present concerns a method of transporting (or delivering) a vector, such as a mutated tyrosine adeno-associated viral expression vector capable of expressing an ototoprotective or an ototoregenerative gene, or a pharmaceutically active agent, across the round window membrane. For this to occur, the permeability of the round window membrane is enhanced to allow transport of the vector or the pharmaceutically active agent across the membrane. The vector or the agent contacts the permeability enhanced membrane, at a sufficient concentration and for a sufficient time, to allow its diffusion and transport thereacross. The vector or the agent is delivered to the inner ear cell where it contacts an inner ear organ, or associated neural structures, of subject so as to treat or prevent the hearing loss.

The permeability of the round window membrane is enhanced by contacting same with a protease or a biocompatible detergent for a time sufficient to cause the round window membrane to become partially disrupted to permit the vector or the pharmaceutically active agent to be transported thereacross.

The permeability of the round window membrane can be enhanced using enzymatic degradation which partially digests the membrane. Proteases such as endopeptidases or exopeptidase that catalyzes the hydrolytic breakdown of proteins into peptides or amino acids are particularly useful. Specific examples of proteases include serine proteases (chymotrypsin, trypsin, elastase); threonine proteases (proteasome hydrolases); cysteine proteases (actinidain, bromelain, calpains, caspases, cathepsins, Mir1-CP, papain); aspartate proteases (cathepsin D, pepsin, chymosin); and metalloproteases (collagenase, elastase, gelatinase), Glutamic acid proteases. Collagenase enzymes are particularly useful examples.

The permeability of the round window membrane may also be enhanced by the use of biocompatible detergent (ionic, nonionic and zwitterionic such as Triton X-100; Triton X-114; NP-40; Brij-35; Brij-58; Tween 20; Tween 80; Octyl glucoside; Octyl thioglucoside; SDS; CHAPS; CHAPSO; Pluronic F-127) or surfactants (Teepol, Lissapol, Alconox) to enhance penetration of the AAV across the RWM. All detergents can be applied at a concentration of 0.1-3% in artificial perilymph composed of (mM) NaCl (127.5), KCl (3.5), NaHCO₃ (25), CaCl₂ (1.3), MgCl₂ (1.2), NaH₂PO₄ (0.75), Glucose (11).

The permeability of the round window membrane may be enhanced by disruption thereof using electroporation or electropermeabilization—a significant increase in the electrical conductivity and permeability of the cell plasma membrane caused by an externally applied electrical field.

Vectors, for example an AAV vector, can be transported across the round window membrane using pore-forming proteins derived from bacteria such as streptolysin-O or tetanolysin is contemplated (Bhakdi et al. Med Microbiol Immunology 1993 182:167-175) or virus such as myristoylated peptide μ1N derived from reovirus outer capsid (Tijana Ivanovic et al. EMBO J. 2008 Apr. 23; 27(8): 1289-1298).

The permeability of the round window membrane is enhanced by contacting same with a solution containing an agent that promotes lipid peroxidation for a time sufficient to cause the round window membrane to become partially disrupted to permit the vector or the pharmaceutically active agent to be transported thereacross.

Application of solutions containing agents that promote lipid peroxidation such hydrogen peroxide, transition metal ions (copper, iron, zinc) or reducing agents (electron donator) to the surface of the round window membrane in artificial perilymph may also be useful to enhance the permeability of the round window membrane.

The permeability of the round window membrane may also be enhanced by irrigating the round window membrane with artificial perilymph with final composition (mM) NaCl (127.5), KCl (3.5), NaHCO₃ (25), CaCl₂ (1.3), MgCl₂ (1.2), NaH₂PO₄ (0.75), Glucose (11) containing benzyl alcohol (10 mg/ml) at a rate of 5 ul/min for 40 min. At the end of irrigation, a matrix (ie gelfoam) containing a vector can be applied to the round window membrane.

The permeability of the round window membrane may also be enhanced by passing air over the RWM causing a mild drying effect (Milkulec et al. Otol Neurotol 2008 Oct. 29(7): 1020-1026). A #3 French suction is placed near the round window niche to avoid direct trauma to the RW membrane, and suction applied for a period of 2 minutes.

To enhance delivery of the vector, a matrix containing the vector can be applied to the round window membrane. The matrix in which the vector can be delivered to the round window membrane can be a synthetic biodegradable polymer composed of polysaccharides (starch, cellulose); protein (gelatin (GELFOAM®), casein, silk, wool); polyesters (polyhydroxyalkanoates); others (lignin, shellac, natural rubber).

Microencapsulation of a pharmaceutically active agent, for example a small molecule therapeutic (a steroid or a neuroprotective compound) in a cationic liposome (an aqueous compartment enclosed by a bimolecular phospholipid membrane consisting of 1-palmitoyl-2-oleolyl-sn-glycero-3-phosphocoline/cholestrylimidazole/dimethyldioctadecylammonium bromide) can be used to enhance delivery of the agent and may be used in conjunction with partial protease degradation/digestion of the round window membrane.

The aforesaid methods of enhancing the permeability of the round window membrane may be used singly or in combination with each other. One example is contemplated in which the round window membrane is partially digested together with any of the above non-protease methods.

### II. Gene Therapy

The present features a method of treating human patients with hearing loss using full length X-linked inhibitor of apoptosis protein (XIAP), a protein that blocks apoptosis. The XIAP can be administered through gene therapy using an adeno-associated viral expression vector encoding XIAP, in which the XIAP is positioned in the vector for expression in the cells of the inner ear organ. The hearing loss is the result of inner ear organ degeneration over time, as is commonplace with aging subjects.

Broadly speaking, the inner ear organ includes both the hearing and the vestibular organs (including the semicircular canals and the otolith organs (utricle and saccule). These organs have hair cells, which include 1) hearing related sensory cells and supporting cells, including outer hair cells; 2) sensory cells and supporting cells and matrix and mechanical structures for sensing vestibular function (both rotation, linear motion and gravity); and 3) associated neural structures and spiral ganglion cells.

In addition to age-related hearing loss, we also contemplate that other types of hearing loss may be treatable using the expression vector described herein. Examples of other types of hearing loss include, for example: 1) ototoxicity caused by chemical or pharmaceutical agents, for example, antineoplastic agents such as cisplatinum or related compounds, aminoglycosides, antineoplastic agents, and other chemical ototoxic agents; 2) noise induced hearing loss, either from acoustic trauma or blast injury; 3) therapeutic radiation; 4) viral infections of the inner ear, such as Herpes Simplex, cytomegalovirus or other viruses or infectious agents (such as Lyme Disease) that can cause inner ear hearing loss; 5) autoimmune inner ear diseases; 6) genetic hearing losses that may have an apoptotic component; 7) inner ear barotrauma such as diving or acute pressure changes; 8) physical trauma such as that caused by head injury, or surgical trauma from surgical intervention in the inner ear; 9) inflammation or other response to administration of other inner ear regenerative compounds or gene therapy techniques; 10) ischemic damage to the inner ear such as in vasculitis, or following acoustic neuroma surgery; and 11) idiopathic or vestibular disorders such as sudden sensurineural hearing loss.

One type of hearing loss that is of interest to us is Usher's syndrome, which is a relatively rare genetic disorder, which is the leading cause of deaf-blindness.

Usher's syndrome is characterized by deafness and a gradual vision loss. The hearing loss is associated with a defective inner ear, whereas the vision loss is associated with retinitis pigmentosa (RP). Usher's syndrome has three clinical subtypes, known as I, II and III. People with Usher I are born profoundly deaf, and begin to lose their vision in the first decade of life. They also exhibit balance difficulties and learn to walk slowly as children, due to problems in their vestibular system. People with Usher II are not born deaf, but do have hearing loss. They do not seem to have noticeable problems with balance; they also begin to lose their vision later (in the second decade of life) and may preserve some vision even into middle age. People with Usher syndrome III are not born deaf, but experience a gradual loss of their hearing and vision; they may or may not have balance difficulties. The hearing impairment associated with Usher syndrome is better understood and is known to be due to damaged hair cells in the cochlea of the inner ear, which inhibit electrical impulses from reaching the brain.

Other syndromes which may exhibit signs similar to Usher syndrome, include Alport syndrome, Alstrom syndrome, Bardet-Biedl syndrome, Cockayne syndrome, spondyloepiphyseal dysplasia congenita, Flynn-Aird syndrome, Friedreich ataxia, Hurler syndrome (MPS-1), Kearns-Sayre syndrome (CPEO), Norrie syndrome, osteopetrosis (Albers-Schonberg disease), Refsum's disease (phytanic acid storage disease), and Zellweger syndrome (cerebro-hepato-renal syndrome).

In addition to treating hearing loss caused by inner ear cell loss using XIAP, we also contemplate that XIAP will also treat or prevent vestibular (balance) organ degeneration. Thus, XIAP gene therapy may be used to slow vestibular organ degeneration associated with aging. Vestibular loss may or may not start at the same time as hearing loss, thus XIAP may be used to simultaneously treat vestibular end organs. Vestibular organ degeneration may also result from trauma or non-trauma to the vestibular organ. Specifically, the vestibular organ degeneration may be due to ototoxicity, viral infections of the inner ear, autoimmune inner ear diseases, recognised inner ear diseases such as Meniere's disease, Delayed Endolymphatic Hydrops, Vestibular neuronitis, Sudden Hearing Loss with Vertigo, and benign positional vertigo; genetic vestibular losses, inner ear barotraumas; or physical trauma, or surgical trauma.

In general, there are two approaches to gene therapy in humans. For in vivo gene therapy, a vector encoding the gene of interest can be administered directly to the patient. Alternatively, in ex vivo gene therapy, cells are removed from the patient and treated with a vector to express the gene of interest. In the ex vivo method of gene therapy, the treated cells are then re-administered to the patient.

Numerous different methods for gene therapy are well known in the art. These methods include, but are not limited to, the use of DNA plasmid vectors as well as DNA and RNA viral vectors. In the present, these vectors are engineered to express XIAP when integrated into patient cells.

Adenoviruses are able to transfect a wide variety of cell types, including non-dividing cells. The discovery includes the use of any one of more than 50 serotypes of adenoviruses that are known in the art, including the most commonly used serotypes for gene therapy: type 2 and type 5. In order to increase the efficacy of gene expression, and prevent the unintended spread of the virus, genetic modifications of adenoviruses have included the deletion of the E1 region, deletion of the E1 region along with deletion of either the E2 or E4 region, or deletion of the entire adenovirus genome except the cis-acting inverted terminal repeats and a packaging signal (Gardlik et al., Med Sci Monit. 11: RA110-121, 2005).

Adeno-associated virus (AAV) vectors can achieve latent infection of a broad range of cell types, exhibiting the desired characteristic of persistent expression of a therapeutic gene in a patient. The discovery includes the use of any appropriate type of adeno-associated virus known in the art including, but not limited to AAV1, AAV2, AAV3, AAV4, AAV5, AAV6 and AAV7 (Lee et al., Biochem J. 387: 1-15, 2005). Previous experiments have shown that genetic modification of the AAV capsid protein can be achieved to direct infection towards a particular tissue type (Lieber, Nature Biotechnology. 21: 1011-1013, 2003). Modified serotype-2 and -8 AAV vectors in which tyrosine residues in the viral envelope have been substituted for alanine residues that cannot be phosphorylated are also contemplated. In the case of tyrosine mutant serotype-2, tyrosine 444 is substitute with alanine (t2 mut 444). In the case of serotype 8, tyrosine 733 is substituted with an alanine reside (t8 mut 733). The titer for t2 mut 444 is 4.89E+12 and that for t8 mut 733 is 7.50E+13.

AAV vectors include those with a mutation of one or more surface-exposed tyrosine residues on capsid proteins. These mutated vectors avoid degradation by the proteasome, and significantly increase the transduction efficiency of these vectors. Mutation of one or more of the tyrosine residues on the outer surface of the capsid proteins including, for example, but not limited to, mutation of Tyr252 to Phe272 (Y252F), Tyr272 to Phe272 (Y272F), Tyr444 to Phe444 (Y444F), Tyr500 to Phe500 (Y500F), Tyr700 to Phe700 (Y700F), Tyr704 to Phe704), Tyr730 to Phe730 (Y730F) and Tyr733 to Phe733 (Y733F) provides improved transduction efficiency of the AAV vectors when compared to wild-type.

The modified vectors may facilitate penetration of the vector across the round window membranes, which would allow for non-invasive delivery of the vectors to the hair cells/spiral ganglion neurons of the cochlea. The EGFR-PTK (epidermal growth factor receptor—protein tyrosine kinase) phosphorylates tyrosine residues on the surface of the capsid targeting them for ubiquitinylation and degradation by the proteosome (Zhong, L, Zhao, W, Wu, J, Li, B, Zolotukhin, S, Govindasamy, L et al. (2007) A dual role of EGFR protein tyrosine kinase signaling in ubiquitination of AAV2 capsids and viral second-strand DNA synthesis. Mol Ther 15: 1323-1330). Using t2 mut 444 or t8 mut 733 it is possible to increase gene transfer by up to 10,000 fold decreasing the amount of AAV necessary to infect the sensory hair cells of the cochlea.

Using ex vivo gene therapy, an individual skilled in the art can be assured that XIAP protein will only be expressed in the desired tissue. In these applications, as well as applications where tissue specific expression of XIAP is not a concern, the above vectors can be constructed to constitutively express XIAP protein. Numerous constitutive regulator elements are well known in the art. Often, elements present in the native viruses described above are used to constitutively express a gene of interest. Other examples of constitutive regulatory elements are the chicken.beta-actin, EF1, EGR1, eIF4A1, FerH, FerL, GAPDH, GRP78, GRP94, HSP70, beta-Kin, ROSA, and ubiquitin B promoters.

For in vivo applications of gene therapy, the above vectors may be modified to include regulatory elements that confine the expression of XIAP to certain tissue types. Numerous examples of regulatory elements specific to certain tissue types are well known in the art. Of particular interest to the discovery are elements that direct gene expression in the hair cells of the cochlea.

In some examples, it may be desirable to direct XIAP expression in an inducible fashion. Several methods of inducible transgene expression are widely used. These methods consist of the transfection of the patient's cells with multiple viral or plasmid vectors. Typically, a first vector expresses the gene of interest under the control of a regulatory element that is responsive to the expression product of a second vector. The activity of this expression product is controlled by the addition of a pharmacological compound or some other exogenous stimulation. Examples of these systems are those that respond to tetracycline, mifepristone, ponasterone A, papamycin, tamoxifen, radiation, and heat shock (Robson et al., J. Biomed. Biotechnol. 2: 110-137, 2003)

We have discovered the protective action of XIAP over-expression in the inner ear in slowing the development of presbycusis, as investigated by using a transgenic mouse in which the expression of human xiap gene is under control of the ubiquitin promoter. Our proof of concept was done using a transgene mouse model, which is engineered to produce XIAP that contains a 6-Myc tag (XIAP-Myc), and is therefore ubiquitously expressed, being present in most cells types in the cochlea. We evaluated levels of both endogenous XIAP (endo-XIAP) and the XIAP-Myc derived from the ub-xiap transgene in different tissues at distinct chronologic intervals to determine if there were any age-related changes in the levels of these proteins, and to examine any potential effects of over-expression of the ub-xiap transgene on the expression of endo-XIAP.

C57BL/6J mice are well known to express early onset (2-3 months of age) and progressive sensorineural hearing loss with aging (Mikaelian, 1979; Henry and Chole, 1980; Willott, 1986; Hunter and Willott, 1987; Li and Borg, 1991; Spongr et al., 1997). Moreover, the aging process of this species appears to be more rapid in the inner ear than in the brain, resulting in “old ears” connected to a young brain at the relatively early-to-middle life span of the animals (Willott, 1986; Parham and Willott, 1988). The reason(s) for this faster aging specifically in the auditory system remains to be explored. Up to three major genes have been identified as major contributors to AHL in mice, and each mouse strain examined may contain one to three of these genes (Erway et al., 1993). For example, a major AHL gene has been mapped in C57BL/6J mice at chromosome 10 and the same gene has been found to be a major contributor to AHL in nine other inbred mouse strains (Erway et al., 1993; Johnson et al., 1997; Johnson et al., 2000). However, it is not known how many different AHL genes are present collectively in each mouse strain and the differences in AHL onset and development across different species cannot be attributed to the allelic heterogeneity of the AHL genes (Johnson et al., 2000). Further, it is not known how these genes are related to the apoptotic cell death seen during AHL.

Previous studies have suggested that apoptosis is involved in degenerative cell death in brain as well as in aged cochleae (Zheng et al., 1998; Alam et al., 2001; Iwai et al., 2001; Spicer and Schulte, 2002; Pickles, 2004). A significant increase of caspase-3 with aging was reported in the organ of Corti, in SGNs, and in the lateral wall of the cochlea in gerbils (Zheng et al., 1998; Alam et al., 2001). The apoptosis pathway can be triggered by various mechanisms in the cochlea of aging gerbils, including accumulated damage from free-radicals and deteriorating mitochondrial function and structure (Zheng et al., 1998). Very recently, induction of apoptotic markers has been correlated with mutations in mitochondrial DNA (mtDNA) accumulated during aging, and with increased markers of oxidative stress (Kujoth et al., 2005). Accumulated mtDNA mutation has long been associated with presbycusis (Seidman et al., 2002; Ohlemiller, 2004). The correlation between the accumulation of mtDNA mutation and apoptotic markers suggests that the mtDNA mutation may promote apoptosis as a cause of cell death during aging (Kujoth et al., 2005). Although AHL is defined as a hearing loss due to aging without significant insults from hazardous factors, it is possible that cochlea presbycusis occurs as the consequence of interplay between hazardous environmental events and genes that govern protection and repair of the cochlea cells (Johnson et al., 2000). For example, several studies have showed that the AHL gene renders C57 mice more susceptible to noise induced hearing loss (NIHL). Nonetheless, it is clear that apoptosis plays an important role for the cochlea lesions accompanying the development of AHL.

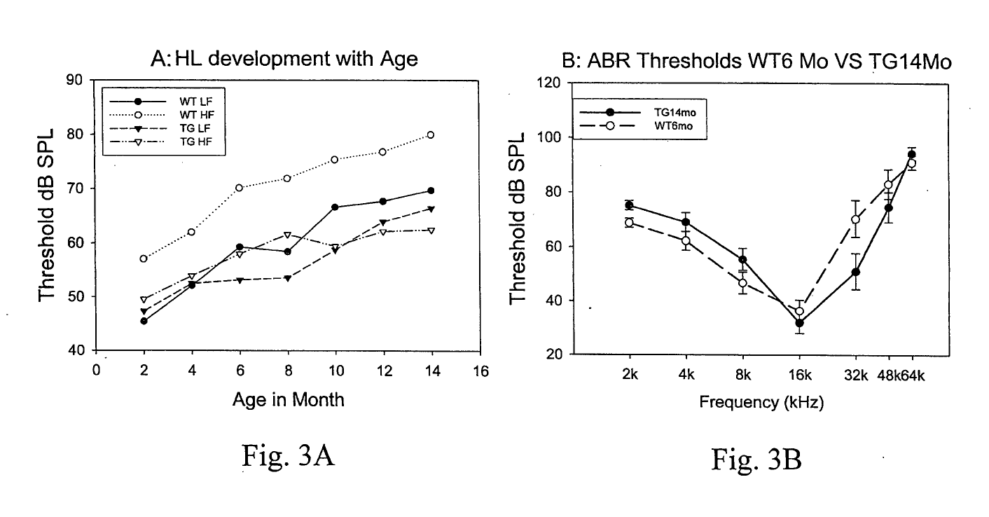

We have discovered that the aging-related hearing loss (AHL) in the C57BL/6J mouse strain can be significantly delayed by the over-expression of XIAP. This protection is particularly apparent at the high-frequency region in which hearing sensitivity in 14 month old TG mice is approximately the same as 6 month WT mice (FIG. 3B). The cytoprotective effect of XIAP is also clearly demonstrated by the significantly less hair cell loss in TG relative to WT mice evaluated at 14 months of age. This result suggests that the cochlea hair cells are dying, at least in part, during the process of aging through apoptosis.

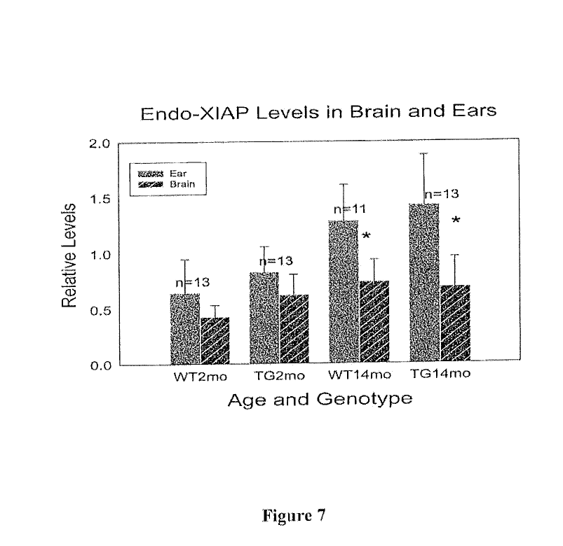

We have also discovered a unique age-related increase in endo-XIAP in the cochlea. This result suggests that the endo-XIAP accumulates in the cochlea in response to the activation of apoptosis with aging. Interestingly, the increasing endo-XIAP with aging was not seen in brain tissue from the same mice, suggesting that apoptosis is more predominant and severe in the cochlea than in the brain. This finding is consistent with the fact that the aging in the cochlea is quicker than in the brain in this strain and provides additional support to the role of apoptosis in AHL. XIAP is thought to be ubiquitously expressed and is translated to produce anti-apoptotic properties in response to a variety of apoptosis-inducing conditions. In contrast, other IAPs show either a more limited expression pattern or inhibit a relatively limited subset of apoptotic triggers. Therefore, a housekeeping function for XIAP may exist. Nevertheless, the level of XIAP in healthy tissue is generally low, as shown in the brain and ears of younger age mice in this experiment (FIG. 7), with an increase in the level of XIAP in response to different stressors. Inferring from the early onset of hearing loss, it appears that apoptosis is triggered more easily in the cochlea than in the brain in this strain, at this stage for known reasons.

We have further discovered that the level of XIAP-Myc, which arises from the transferred human xiap gene, appears to remain unchanged with age. This indicates that transgene may not be regulated in response to the apoptosis accompanying AHL. Rather, a stabilized expression is provided by the transgene. Without wishing to be bound by theory, we believe that it is this age-stabilized XIAP that provides the extra protection against apoptosis in the transgenic mice; and this stabilized expression does not suppress the expression of endogenous xiap gene, based on the fact that the endo-XIAP levels in the cochlea appears to be similar in the two genotype groups. It also appears that the endo-XIAP, despite its increase with aging, is not adequate to fully protect the cochlea from apoptotic cell death. The extra quantity of XIAP provided by the transgene seems to help to protect the cochlea from aging.

Although great efforts have been made to understand the biological control of XIAP expression, it is still not entirely clear how XIAP expression is regulated in responses to various insults during apoptosis. XIAP has been found to be under transcriptional control by the stress-inducible transcriptional activator NF-κB (Stehlik et al., 1998) and to be regulated at the level of protein synthesis by ubiquitination (post-translation regulating). However, it is also possible that transcription of the gene is promiscuous (Holcik, 2003), and that regulation is mostly post-transcriptional to allow for differential expression in tissues that require more or less XIAP protein. This possibility is supported by the extensive 5- and 3-untranslated regions (UTRs) in the messenger RNA of XIAP and the internal ribosome entry site (IRES) in the 5-UTR (Holcik et al., 1999). Several binding proteins have been described which regulate XIAP expression at the RNA level (see review by (Holcik, 2003)). It has been suggested that the IRES-mediated translation allows for enhanced expression of XIAP when cap-dependent translation protein synthesis is shut off or compromised following the induction of apoptosis (Hellen and Sarnow, 2001). Increased expression of endo-XIAP has been reported in various conditions of cellular stress (Holcik et al., 1999; Holcik et al., 2000a; Holcik et al, 2000b). XIAP activity has also been reported to be under the control of two negative regulators, termed XIAP associated factor 1 (XAF1) (Fong et al., 2000; Liston et al., 2001) and direct IAP binding protein with low pl (Smac/DIABLO) (Du et al., 2000; Verhagen et al., 2000) at post-translation level.

Because only the coding domains of the human xiap gene are transferred in the XIAP-Myc mice, it is possible that the finding of stabilized XIAP-Myc with aging is due to the lack of regulating sites in the mRNA from the transgene. The stabilized expression of XIAP-Myc with aging also suggests that this expression is not under the control of the two negative regulators mentioned above. In a recent study using viral vector for xiap gene transfection, it was suggested that the exogenous XIAP exerted its hair cell protection against cisplatin ototoxicity mainly by combining with one of the two negative regulators, namely Smac/DIABLO, based on the finding that the transgene modified to not contain the binding site for Smac/DIABLO did not provide this protection (Chan et al., 2007). This study suggests that the binding of exogenous XIAP with Smac/DIABLO frees the endo-XIAP to counteract apoptosis. However, this is unlikely to be the mechanism of protection provided by the XIAP-Myc in the aging cochleae in our experiments. Since there is no mechanism for the up-regulation of XIAP-Myc, binding with the negative regulator would likely exhaust this exogenous XIAP. However, we did not see an age-related decrease in XIAP-Myc level. The increase of endo-XIAP with age is likely due to the positive regulation through internal ribosome entry site (IRES) in the 5-UTR that occurs at a post-transcriptional level. Nevertheless, the total level of XIAP is increased with the addition of XIAP-Myc in the cochleae of in the TG group. This increase appears to be effective in delaying aging in the cochlea in this species.

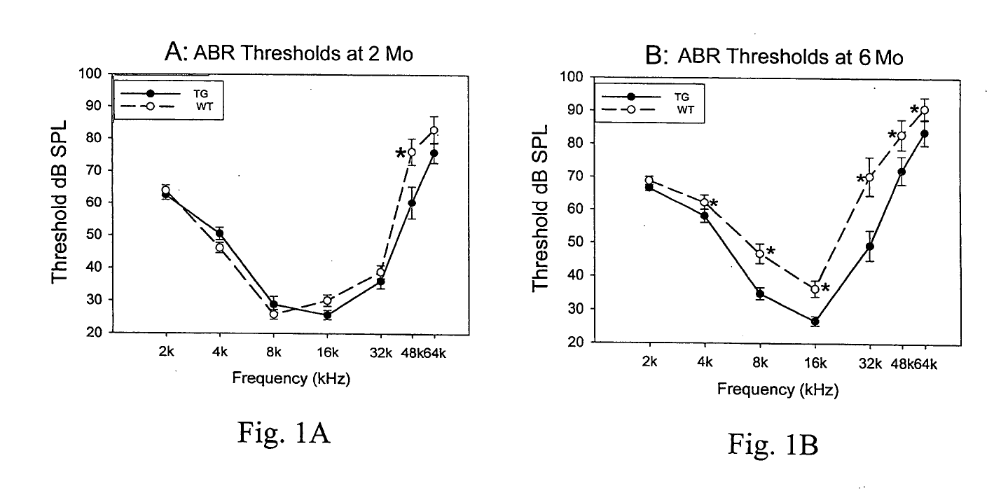

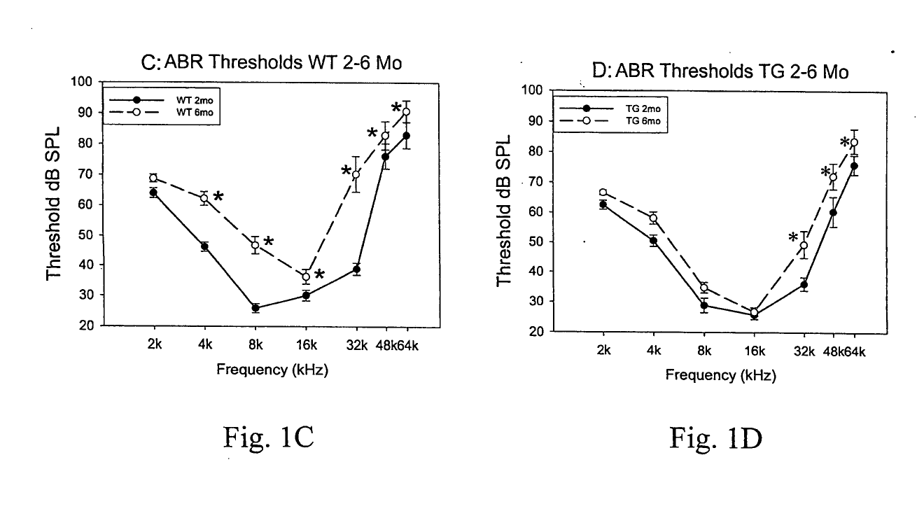

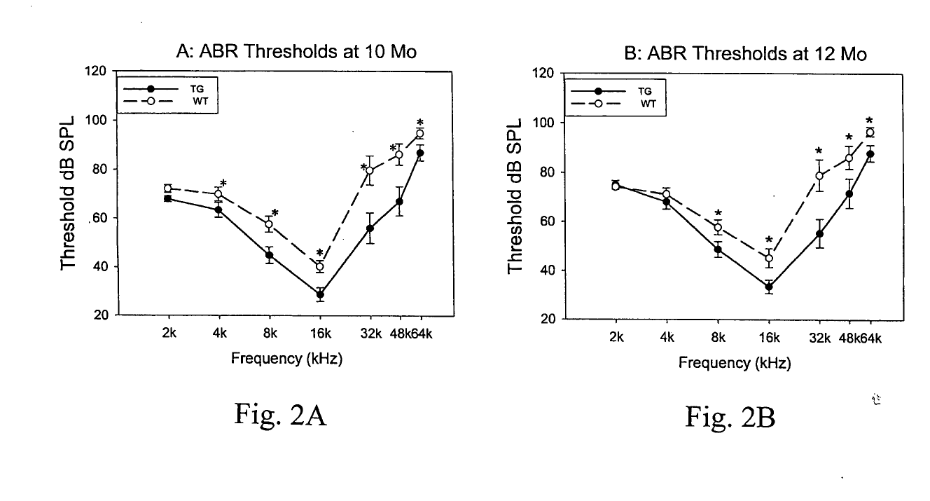

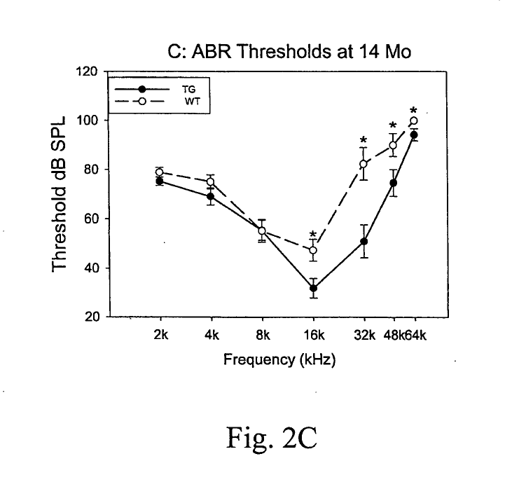

We have also discovered that XIAP-Myc provides better protection at the high-frequency region than in the low frequency region. Consistent with previous reports (Mikaelian, 1979; Li and Borg, 1991; McFadden et al., 2001), our ABR results show that the age-related hearing loss starts at the high-frequency end of the hearing range of the mice, and spreads out towards middle and low frequency regions with aging. A significant hearing loss in the low-to-middle frequency regions has also been reported in the previous studies and is confirmed in the present study. To our surprise, we found that the LF hearing loss is not a result of a downward spreading from the high-frequency regions, but rather is separately initiated in the low-frequency regions. This LF hearing loss was evident even when comparing the hearing of 2 and 4 month old mice, especially in WT groups (FIG. 1). The hearing loss in this region developed slightly later and progressed much slower in the TG group than in the WT group up to 8 months of age (FIG. 3A). But the LF loss was significant by the end of the experiment in both genotype groups. The LF hearing loss appears to accelerate after 8 months of age in TG groups so that there is no statistical difference between the two groups (FIGS. 2 and 3). More importantly, the amount of hair cell loss appears to match the degree of hearing loss in the high frequency region in the two groups. In the low frequency region, however, we generally see no hair cell loss in the TG group and only a slight hair cell loss in the WT group. Therefore, the LF hearing loss is not due to hair cell loss. A similar discrepancy between the hair cell loss and the elevation of the thresholds has also been demonstrated in previous studies (Spongr et al., 1997; McFadden et al., 2001). Since we did not observe pathology in other part of the cochleae, we do not know which degenerative changes are responsible for the LF hearing loss shown in this species during aging. Since the LF hearing loss is not protected by XIAP over-expression, the pathology or degenerative changes involved may not be due to apoptosis.

In the predominant conceptual framework for AHL or presbycusis proposed by Schuknecht (Schuknecht, 1964; Schuknecht and Gacek, 1993), the three major cochlea elements (organ of Corti, SGNs, and Stria vascularis (SV)) can degenerate separately, thereby contributing to AHL independently. The apical turn of the cochlea has been found to be prone to primary SGN loss in humans and many animal species including C57 mice (Covell and Rogers, 1957; Keithley et al., 1992; Felder and Schrott-Fischer, 1995; Dazert et al., 1996; Willott et al., 1998; Ohlemiller and Gagnon, 2004b). In some recent reports, pathology related to the SGN loss have been reported to show apical-to-basal gradient during the development of presbycusis (Ohlemiller, 2004; Ohlemiller and Gagnon, 2004a, 2004b). This is opposite to the HC loss that is develops from the basal turn to the apex. These include the abnormalities of the spiral limbus, pillar cells and Reissner's membrane. However, it is not clear how these changes are related to the death of SGNs and if these changes are due to apoptosis.

Even though we were unable to examine the anatomy of the SV and SGNs, we think that a postulated non-apoptotic lesion is more likely to occur in the strial vascularis (SV), but not in the SGNs. This is supported by the previous studies which suggest that apoptosis is always involved in aging related SGN death, while the degenerative changes in the SV can be atrophic in nature. In one report, for example, DNA fragmentation (an indicator of an endonuclease activation seen in apoptosis) was found predominantly in the OHCs and SGNs, but not in the stria cells, which showed a marked atrophy (Zheng et al., 1998). A later study reported that the expression of a particular protein in the apoptotic pathway (the caspase-3p20) increased with aging in the organ of Corti, SGNs, as well as lateral wall of the cochlea in gerbils. This increase in active caspase was claimed to be compatible with functional hearing deterioration (Alam et al., 2001). To our knowledge, data about the topographic quantification of degenerative changes in the SV is not available in the C57 species. In one study, SV atrophy was presented at the basal turn but not at the apical low-frequency region where the discrepancy occurred (Mikaelian, 1979).

Another possible explanation for the LF hearing loss that does not involve the HC is conductive hearing loss from middle ear pathology which is often low-frequencies biased. Although we did not see any obvious middle ear abnormalities or fluid by visual inspection when cochleae were harvested at the end of the experiments, this simple inspection cannot rule out entirely the possibility of aging related changes in the middle ear structures.

We have therefore demonstrated that over-expression of XIAP by genetic manipulation provides protection in C57 mice against age-related hearing loss, and this loss is probably is a result of the accumulation of apoptotic processes in the cochlea with aging. The transferred XIAP gene is not regulated in response to apoptosis, rather, it provides a steady baseline activity throughout the mouse's life span. The transferred gene does not interfere with the expression of the endo-XIAP gene. The endo-XIAP gene expression increases with aging in the cochlea but not in the brain of C57 mice, suggesting that the cochlea is the more predominant site for apoptosis in this species. Over-expression of XIAP provides protection against AHL in the high-frequency region but not in the low frequency region where the degenerative pathology may not be apoptotic, or inner ear related.

Our evidence suggest that apoptosis plays a significant role in age-related hearing loss (AHL) or presbycusis. We evaluated whether over-expression of the anti-apoptotic protein known as X-linked Inhibitor of Apoptosis protein (XIAP) slows the development of presbycusis. We compared the effect of aging on the hearing status of transgenic (TG) mice that over-express human xiap under control of the ubiquitin promoter on a pure C57BI/6 genetic background with wild-type (WT) littermates. In order to distinguish endogenous XIAP from that derived from the transgene, the transgenic XIAP was engineered to contain a 6-Myc tag (XIAP-Myc). Auditory brainstem responses (ABR) were measured every two months from 2 to 14 months of age. Hair cell loss in the cochlea was assessed by cochleograms following the final ABR testing. Comparison of the levels of endogenous XIAP (endo-XIAP) and XIAP-Myc over time demonstrated that the transgene elevated total XIAP by up to 50% in the cochlea and by 100% in the brain, and that XIAP-Myc level did not change with aging in either tissue. In contrast, endo-XIAP appears to increase with aging in the cochlea, but not in the brain. ABR measurements showed that WT mice developed hearing loss much faster than XIAP-Myc mice. XIAP over-expression reduced hearing loss associated with aging, particularly within the high-frequency range. The average total hair cell loss per-cochlea was 665.47±417.99 (mean±SD) in the WT group compared to 219.95±258.37 in the TG group (t-test, t=−4.221, p<0.001). Taken together, these results suggest that XIAP over-expression reduces age-related hearing loss and hair cell death in the cochlea. Treatment strategies based on elevation of XIAP may, therefore, have utility in the treatment of presbycusis.

Exposure to hazardous noise is the primary cause of hearing impairment in North America (Daniel, 2007). Noise induced hearing loss (NIHL) is thought to result primarily from damage to the organ of Corti (OC) in which hair cells, mainly outer hair cells (OHCs), are considered the most vulnerable (Bohne, 1976). Although OHC loss is a contributing factor to NIHL, hearing deficits are not closely correlated with the number of missing OHCs suggesting the death of other cell types in the cochlea play an important role in the development of NIHL (Chen et al., 2007; Liberman, 1990; Wang et al., 2002). For example, loss of spiral ganglion neurons (SGN) (Kujawa et al., 2008) and cell death in structures outside the OC such as the stria vascularis (SV) may also contribute to NIHL (Hukee et al., 1985; Masutani et al., 1995; Smith et al., 1985; Suzuki et al., 2002; Yamane et al., 1991). Both necrosis and apoptosis have been implicated in NIHL (Bohne et al., 2007; Nicotera et al., 2003; Yang et al., 2004). Their relative contribution to cochlear cell death appears to be a function of noise severity and distance from the region of the OC that is most severely damaged (Bohne et al., 2007; Hu et al., 2000; Nicotera et al., 2003; Yang et al., 2004). Nevertheless, apoptosis is thought to play a significant role in noise-induced damage of the cochlea (Hu et al., 2002; Hu et al., 2006; Yang et al., 2004).

Apoptosis may be executed by a family of cysteine proteases called caspases (Eldadah et al., 2000; Miller, 1997; Nicholson et al., 1997). To date 14 caspases have been identified that are broadly divided into two groups, initiator (caspase-6, -8, -9 and -10) and executioner (caspase-2, -3 and -7) (Eldadah et al., 2000; Van De Water et al., 2004). In NIHL, apoptosis resulting from activation of the extrinsic pathway (caspase-8) or the intrinsic pathway (caspase-9) has been observed (Zimmermann et al., 2001). These two pathways converge on caspase-3, the final executioner of apoptosis. Both the intrinsic and extrinsic pathways are inhibited by members of the inhibitor of apoptosis protein (IAPs) family (Deveraux et al., 1997; Deveraux et al., 1998; Roy et al., 1997). The IAP family consists of 8 members that all have at least one baculoviral of apoptosis repeat (BIR) domain. The importance of the IAPs for cell survival is indicated by a reduction of the apoptotic threshold when they are down-regulated (Ishigaki et al., 2002; Kominsky et al., 2002) and an increase when over-expressed (Schoemaker et al., 2002; Simons et al., 1999; Suzuki et al., 2001). Among the IAP family members, X-linked inhibitor of apoptosis (XIAP) is considered to be the most potent due to its ability to potently suppress caspase-3 activity (Deveraux et al., 1999a; Deveraux et al., 1999b). In the case of in vivo models for stroke and Parkinson's disease, XIAP over-expression not only renders neurons more resistant to cell death but preserves normal function (Crocker et al., 2003; Trapp et al., 2003; Xu et al., 1999).

The C57BL/6J mouse strain is known for its rapid development of hearing loss with age (Briner et al., 1989; Spongr et al., 1997a; Spongr et al., 1997b; Willott et al., 1988). Using C57BL/6J mice that over-express human XIAP under the control of the ubiquitin promoter (ubXIAP), we found that by comparison to wild-type littermates, ubXIAP mice showed a delay in the development of presbycusis that was associated with a reduction of cochlear hair cell loss (Wang et al., 2008). We used these transgenic mice to determine the effects of XIAP over-expression on NIHL and associated cochlear damage.

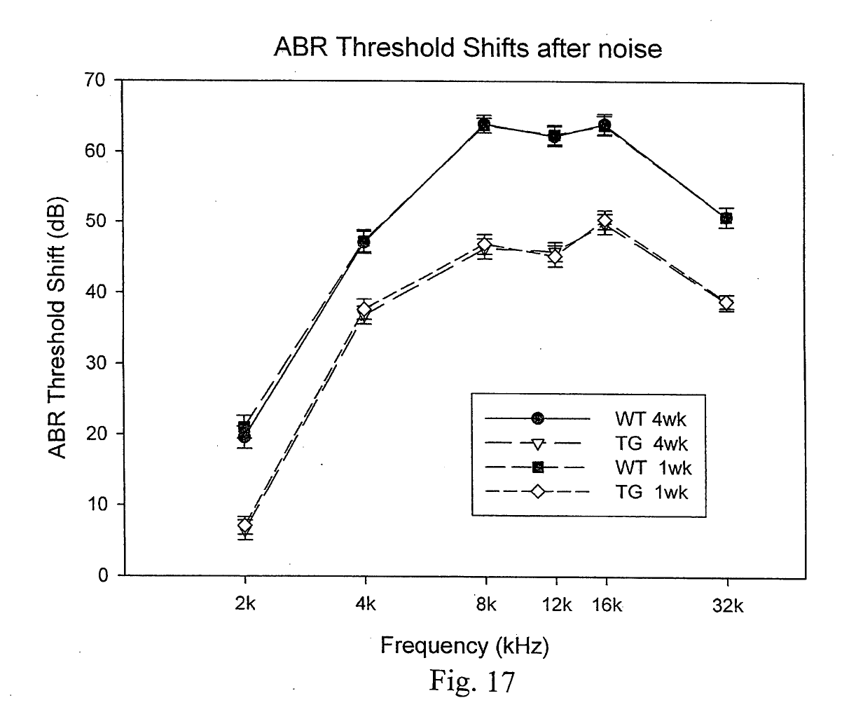

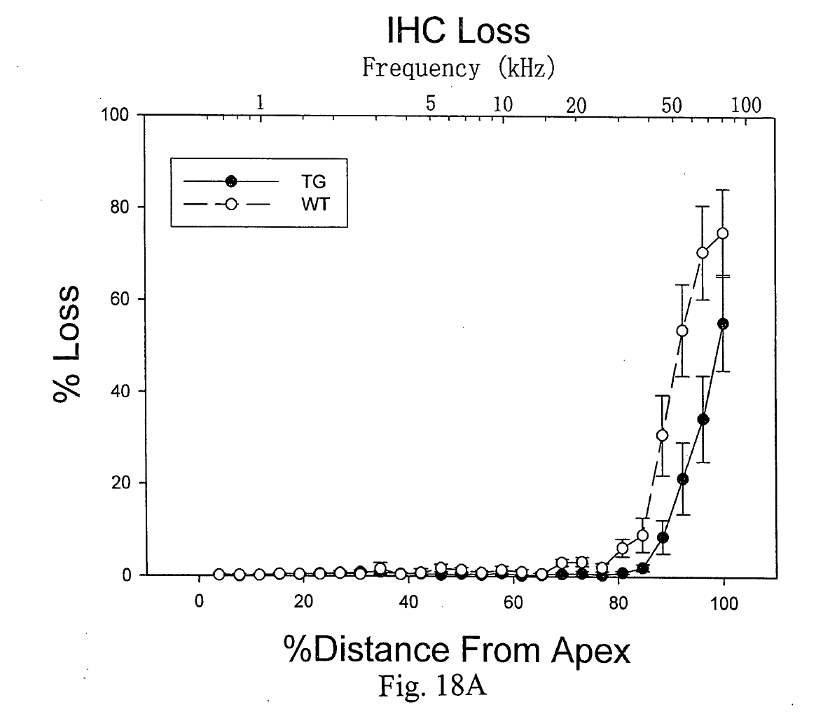

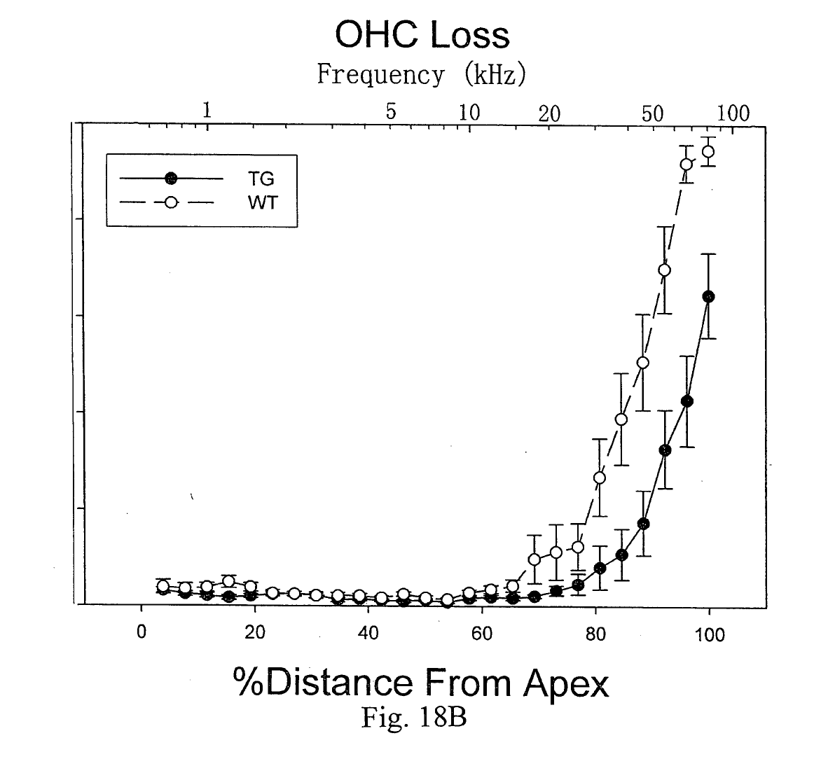

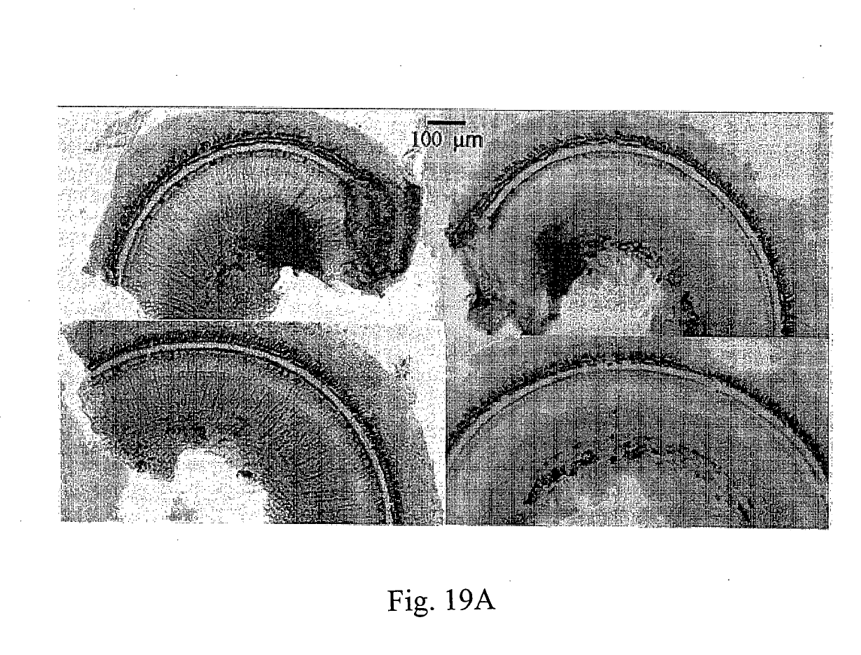

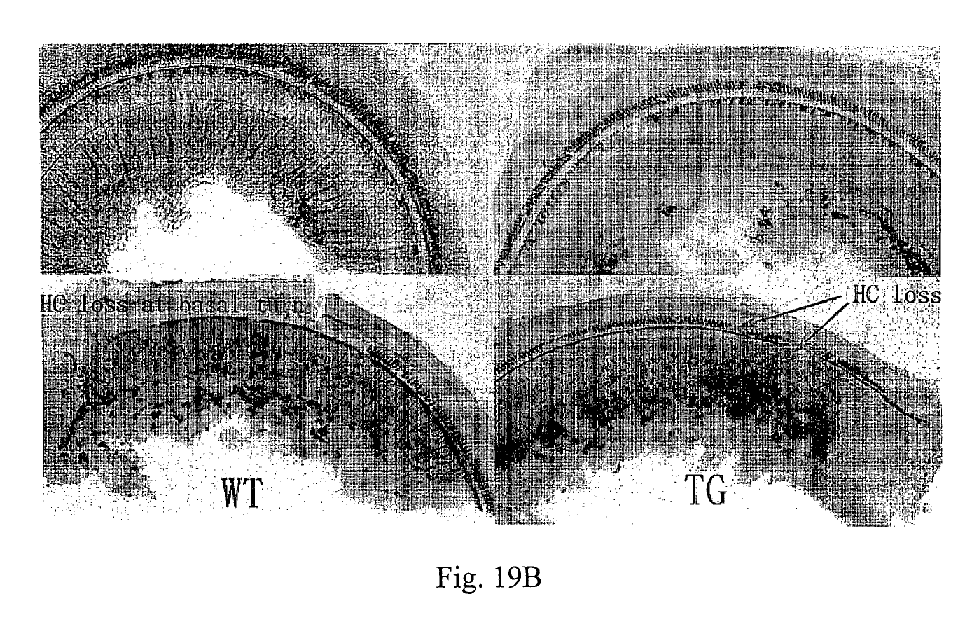

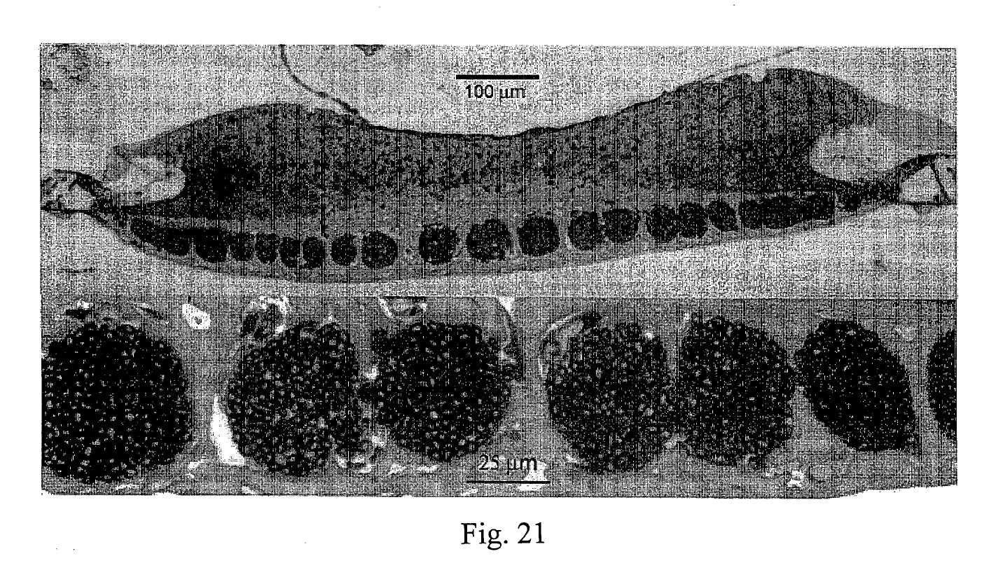

We have demonstrated that the over-expression of XIAP through the ubxiap transgene significantly mitigated the degree of NIHL in C57BL/6J mice. The WT mice developed greater threshold shifts after the noise trauma than the TG mice throughout the frequency range tested, except at 64 kHz at which the ceiling effect confounded the results (FIG. 17). Correspondingly, the hair cell loss (FIGS. 18 and 19) and the lesion to the SGNs and their dendrites to the HCs was significantly less in the TG groups (FIGS. 21, 22 and 23).

These results support the hypothesis that XIAP over-expression in C57BL/6J mice can provide a significant protection to the cochlea against noise induced lesions. However, the limits of this protection against NIHL were evident by the still significant cochlear dysfunction and pathologies noted in the TG group.

There are several potential reasons for this limitation in protection. First of all, the hearing loss induced by noise is not fully attributed to the loss of hair cells that died through apoptosis, and other mechanisms of damage are beyond the protection of XIAP. In addition to the fact that HCs may die through non-apoptotic pathways, lesions on other loci around HCs may cause hearing loss. Discrepancies between the actual amount and distribution of HC (especially OHC) death and the threshold shifts have been well reported (Borg, 1987; Chen et al., 2003; Chen et al., 2005; Kujawa et al., 2006; Liberman, 1990). Non-lethal damages to the HCs and accessory structures in the OC have long been considered to play accessory roles in NIHL. One of the most likely loci for this is the stereocilia and tip links (Canlon, 1988; Kujawa et al., 2006; Liberman, 1990; Wang et al., 2002). Another possibility is damage to the lateral walls of the OHCs, especially structures related to the function of prestin (Chen, 2006; Chen et al., 2003; Chen et al., 2005; Chen et al., 2007; Zhao et al., 1999), the motor protein critical for the function of OHCs as a mechanical amplifier (Liu et al., 2003; Zheng et al., 2003a; Zheng et al., 2000; Zheng et al., 2003b).

The noise used in this experiment was intense and produced a wide-spread threshold shift, although larger threshold shifts were seen above 4 kHz. At the lowest frequency tested (2 kHz), the threshold shifts in both groups were small. Such deterioration in hearing function across a large frequency area is more likely due to the damage to structures for cochlear transduction and mechanical amplification. Such damage may not initiate apoptosis and therefore beyond the scope of the protection exerted by XIAP over-expression. In the present study, we did not assess these more subtle indicators of damage in this study, since we our focus was on cell death.

Data in several recent reports and herein show wide spread noise induced lesions in the 8^{th} N terminal and SGN loss in mice (Kujawa et al., 2006; Kujawa et al., 2008; Wang et al., 2002). This is interesting because the damage to the dendrites of SGNs, and even death of SGNs, were seen in regions of the cochlea where there was no significant HC loss and limited or no functional hearing loss. However, more experiments are needed to see if such lesion can occur in other species with bigger cochleae. In the present study, a similar loss in 8^{th} N fibers was found one month after the noise exposure. However, the partial loss of SGNs may not be responsible for the threshold shift. Measurements taken at this time identified a smaller loss of SGNs than of nerve fibers. More SGN loss is expected at longer intervals after noise exposure, according to previously published data (Kujawa et al., 2008).

A second possible reason for the limited protection in this transgenic model is due to the limited elevation of the XIAP level and potential interaction between the ubXIAP and the endo-XIAP. The exogenous xiap gene promoted by ubiquitin resulted in a roughly 50 increase of total XIAP level in the cochlea in the control condition. Since apoptosis is an important physiological mechanism for controlling early development, it would be a concern to increase the XIAP too highly. Otherwise, animals may not go through normal development. In addition, the level of the ubXIAP was constant due to the lack of regulating zones in the transgene and therefore it did not respond to the stress of noise. Furthermore, the noise-induced elevation of endo-XIAP level is relatively small in the TG group, indicating potentially a negative feedback from the ubXIAP to the regulating mechanism for the endo-XIAP

The noise exposure used in this experiment is relatively intense. It is possible that the internal protective mechanisms mediated by XIAP are not activated promptly enough to protect the cochlear cells. Therefore, a constant high level of exogenous XIAP may be required for an ideal protection. This can be achieved by local gene transfection, thus avoiding the potential impact on development and other side effects (such as the tumour growth) of transgenic manipulation at genome level.

In conclusion, we have demonstrated that C57BL/6J mice carrying ub-XIAP provides strong but not complete protection again NIHL and a reduction in measured damage to the cochlea. This protection resulted in smaller threshold shifts and a reduction in the loss of IHCs, OHCs, SGNs and auditory nerve fibers in TG mice. The shifts in auditory thresholds were present across the entire frequency range; however, HC damage was restricted to the basal end of the cochlea demonstrating an indirect relationship between HO loss and the functional measures. An elevation of the endo-XIAP level was found in western blot, while the ubXIAP level remained to be unchanged.

In addition to the loss of hair cells and spiral ganglion neurons in the cochleae, degeneration of the strial vasularis and spiral ligament may also contribute to aging-related hearing loss (presbycusis) (Ohlemiller, K. K. (2009) Brain Research 1277: 70-83). The degeneration of strial vascularis and spiral ligament is largely due to apoptosis (Spicer, S. S. and B. A. Schulte, Spiral ligament pathology in quiet-aged gerbils. Hear Res, 2002. 172(1-2): 172-85; Zheng, Y., et al., Endonuclease cleavage of DNA in the aged cochlea of Mongolian gerbil. Hear Res, 1998. 126(1-2): 11-18) and can therefore can be prevented or delayed by XIAP-based gene therapy.

### III. Pharmaceutical Compositions

The vector used with some embodiments as described herein can be incorporated into pharmaceutical compositions suitable for administration to a subject. In some particular embodiments, the pharmaceutical composition comprises the vectors described herein and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it can be preferable to include isotonic agents, for example, sugars, polyalcohols such as mannitol, sorbitol, or sodium chloride in the composition. Pharmaceutically acceptable carriers can further comprise minor amounts of auxiliary substances such as wetting or emulsifying agents, preservatives or buffers, which enhance the shelf life or effectiveness of the vector or pharmaceutical composition.

The compositions described herein may be in a variety of forms. These include, for example, liquid, semi-solid and solid dosage forms, such as liquid solutions (e.g., injectable and infusible solutions), dispersions or suspensions, tablets, pills, powders, liposomes and suppositories. The form used depends on the intended mode of administration and therapeutic application. Typical compositions are in the form of injectable or infusible solutions, such as compositions similar to those used for passive immunization of humans. The typical mode of administration is intratympanic (in the middle ear), intracochlear, parenteral (e.g., intravenous, subcutaneous, intraperitoneal, intramuscular, intrathecal). In one example, the vector is administered by intravenous infusion or injection. In another example, the vector is administered by intramuscular or subcutaneous injection. In another example, the vector is administered perorally. In yet another example, the vector is delivered to a specific location using stereostatic delivery, particularly through the tympanic membrane or mastoid into the middle ear.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the vector in the required amount in an appropriate solvent with one or a combination of ingredients enumerated above, as required, followed by filtered sterilization.

Generally, dispersions are prepared by incorporating the vector into a sterile vehicle that contains a basic dispersion medium and the required other ingredients from those enumerated above. In the case of sterile lyophilized powders for the preparation of sterile injectable solutions, the preferred methods of preparation are vacuum drying and spray-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be achieved by including an agent in the composition that delays absorption, for example, monostearate salts and gelatin.

The vectors described herein can be administered by a variety of methods known in the art. As will be appreciated by the skilled artisan, the route and/or mode of administration will vary depending upon the desired results. In certain embodiments, the vector may be prepared with a carrier that will protect the vector against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are generally known to those skilled in the art.

The pharmaceutical compositions described herein can include a “therapeutically effective amount” or a “prophylactically effective amount” of the vectors described herein. A “therapeutically effective amount” refers to an amount effective, at dosages and for periods of time necessary, to achieve the desired therapeutic result, in this case for both prophylaxis and treatment of hearing loss or impairment of balance without unacceptable toxicity or undesirable side effects.

A therapeutically effective amount of the vector can vary according to factors such as the disease state, age, sex, and weight of the individual, and the ability of the vector to elicit a desired response in the individual. A therapeutically effective amount can also be one in which any toxic or detrimental effects of the vector are outweighed by the therapeutically beneficial effects. A “prophylactically effective amount” refers to an amount effective, at dosages and for periods of time necessary, to achieve the desired prophylactic result. Typically, since a prophylactic dose can be used in subjects prior to or at an earlier stage of disease, the prophylactically effective amount can be less than the therapeutically effective amount.

Dosage regimens can be adjusted to provide the optimum desired response (e.g., a therapeutic or prophylactic response). For example, a single bolus can be administered, several divided doses can be administered over time or the dose can be proportionally reduced or increased as indicated by the exigencies of the therapeutic situation. It can be especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of vector calculated to produce the desired therapeutic effect in association with the required pharmaceutical carrier. The specification for the dosage unit forms can be dictated by and directly dependent on (a) the unique characteristics of the vector and the particular therapeutic or prophylactic effect to be achieved, and (b) the limitations inherent in the art of formulating such vector for treating or preventing hearing loss or impaired balance in a subject.

It is known that in the auditory system, three major viral vectors have been investigated for cochlear gene transfection: (1) lentivirus, (2) adenovirus and (3) Adeno-associated virus (AAV). The gene transfected by adenovirus vector has limited expression time and the vector has been associated with adverse immune reactions (Staecker, Brough, Praetorius, & Baker, 2004). The lentivirus vector, although capable of maintaining long term expression, is particularly suited for targeting neurons, but not hair cells (Federico, 1999). Since the AAV vector has several advantages such as long lasting expression of synthesized genes (Cooper et al, 2006), and low risk for pathogenic reactions (because they are artificially manufactured and not ototoxic) (Kaplitt et al., 1994), it is likely to be the best choice of viral vector for cochlear protection by gene therapy.

Cochlear gene transfection in animals has utilized several approaches for vector delivery: (1) direct injection through round window membrane (RWM) into the perilymph, (2) intracochlear infusion through cochleostomy, and (3) transfusion through an intact RWM (Aarnisalo, Aarnisalo, Pietola, Wahlfors, & Jero, 2006). The third approach (transfusion through intact RWM) is least invasive and most likely to be accepted in human application. Until now, it was known that RWM is not permeable to AAV (Jero et al, 2001).

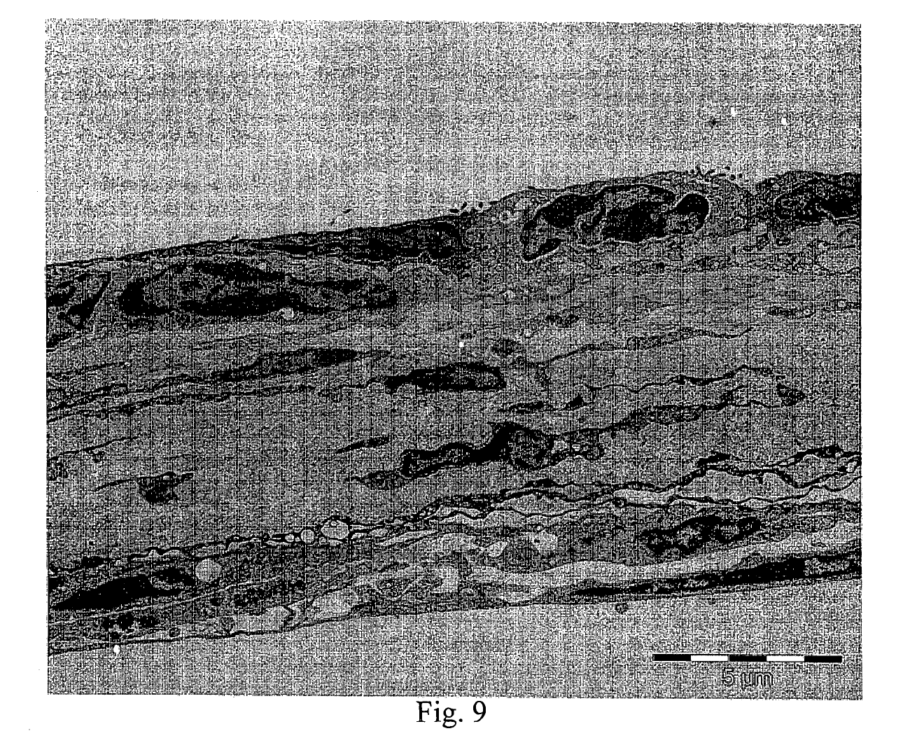

The intact RWM consists of three layers: two epithelia layers separated by a layer of connective tissue (FIG. 9), with collagen being a major component of the RWM. We have now demonstrated that the permeability of RWM can be increased temporarily by digestion of the membrane with collagenase. We then investigated whether (1) the digestion of RWM with the enzyme could facilitate the gene transfection of inner ear cells, and (2) if the digestion was safe to inner ear function and the structure of RWM.

### EXAMPLES

The following examples are designed to illustrate the discovery and are not to be construed as limiting in any way.

The following is a list of Abbreviations used hereinabove and hereinbelow
ABR—Auditory Brainstem Response
ANOVA—Analysis of Variance
ATP—Adenosine Triphosphate
HC—Hair Cell
IAP—Inhibitor of Apoptosis Protein
IHC—Inner Hair Cell
OHC—Outer Hair Cell
NIHL—Noise Induced Hearing Loss
OC—Organ of Corti
ROS—Reactive Oxygen Species
SGN—Spiral Ganglion Neuron
SL—Spiral Ligament
SV—Stria Vascularis
TG—Transgenic
TNF—Tumour Necrosis Family
UB-XIAP—Ubiquitous X-linked Inhibitor of Apoptosis Protein
WT—Wild Type
XIAP—X-linked Inhibitor of Apoptosis Protein

### 1: Transgenic Mouse and Expression Vector Production

Transgenic founders were generated by microinjection of a linearized plasmid construct consisting of the Ubiquitin C promoter, 6 repeats of the 9E10 myc epitope tag fused to the amino terminus of the human XIAP coding region, and a polyadenylation signal from SV40. The construct was microinjected into the male pronucleus of C57BI/6 X C3H F1 zygotes. All lines were maintained in the heterozygous state by cross breeding with wild type C57BI/6 mice. Transgene status within the colony was determined by PCR targeting 6-myc tag.

The XIAP-Myc C57 transgenic (TG) mice and wild-type (WT) littermates were bred in the animal facility at Dalhousie University. In total, 48 mice were recruited into this study for longitudinal observation of the development of hearing loss with time. There were 24 in each of the WT and TG groups with matched number of mice of each gender in the two groups. During the 14 months of observation, some mice died for various reasons. At the end of the experiment, 17 TG and 15 WT mice survived. Hearing status was evaluated using frequency-specific auditory brainstem responses (ABR) that were performed every two months from the ages of 2 months to 14 months. After the final ABR testing, the animals were sacrificed and the both cochleae were harvested; one was used for evaluation of hair cell loss and the other for the quantification of XIAPs. In total, 19 cochleae were taken from each group for cytocochleograms. Western blotting was employed for the quantification of both XIAP-Myc and endogenous XIAP. A piece of brain tissue was also taken from each animal for XIAP testing as well. Western blotting was successful in 14 cochleae in the TG group and 11 in the WT group. Correspondingly, Western blotting was performed using brain tissue from the same mice in each group. To investigate in more detail the effects of aging on XIAP expression, an additional 34 young mice (2 months old, 17 in each genotype group) were recruited to evaluate XIAP expression with Western blotting.

For administration of a vector to treat age-related hearing loss, the adeno-associated virus serotype 2 (AAV-2) construct is used. The adeno-associated virus serotype 2 (AAV-2) construct includes a myosin7a promoter to drive expression of XIAP in the outer hair cells that are necessary for high frequency hearing and lost with aging. It should be noted that outer hair cell loss and inner hair cell loss can be targeted using the vector. Moreover, the expression promoter may be ubiquitin. This is done in two ways 1) by direct injection of the AAV2-1-XIAP using saline as a vehicle, as a composition, into the cochlea or by simply injecting this construct past the tympanic membrane so that it will be taken up by the round window membrane of the cochlea in sufficient amounts to infect the outer hair cells. In other methods, the agent may be applied onto an absorbable material such as Gelfoam® that is placed against the round window, and delivers the vector to the round window. In other methods of delivery, an active controlled release pump is used to direct the agent in solution at a predefined rate to the round window area. In other methods of delivery, a passive wick is placed against the round window membrane, and the agent is applied to the lateral end of this wick for delivery by wicking action to the round window membrane. Direct routes to the cochlea may include a fenestra into the stapes footplate, round window membrane, labyrinth (semicircular canals), promontory, or via the internal auditory canal through CSF and neural pathways.

MV Vector preparations are produced by the plasmid cotransfection method [S. Zolotukhin et. al. Gene therapy 1999]. Briefly, one cell factory (Nalgene Nunc International, Rochester, N.Y., USA) with approximately 1×10⁹ HEK 293 cells is cultured in Dulbecco's Modified Eagle's Medium supplemented with 5% fetal bovine serum and antibiotics (cDMEM). A CaPO4 transfection precipitation is set up by mixing a 1:1 molar ratio of rAAV vector plasmid DNA and serotype specific rep-cap helper plasmid DNA. This precipitate is added to 1100 mL of cDMEM and the mixture is applied to the cell monolayer. The transfection is allowed to incubate at 37° C. for 60 h. The cells are then harvested and lysed by three freeze/thaw cycles. The crude lysate is clarified by centrifugation and the resulting vector-containing supernatant is divided among four discontinuous iodixanol step gradients. The gradients are centrifuged at 350,000 g for 1 h, and 5 ml of the 60-40% step interface is removed from each gradient and combined.

This iodixanol fraction is further purified and concentrated by column chromatography on a 5-ml HiTrap Q Sepharose column using a Pharmacia AKTA FPLC system (Amersham Biosciences, Piscataway, N.J., USA). The vector is eluted from the column using 215 mM NaCl, pH 8.0, and the rAAV peak collected. Vector-containing fractions are then concentrated and buffer exchanged in Alcon BSS with 0.014% Tween 20, using a Biomax 100K concentrator (Millipore, Billerica, Mass., USA). Vector is then titered for DNase-resistant vector genomes by Real-Time PCR relative to a standard. Finally, the purity of the vector is validated by silver-stained SDS-PAGE (the three AAV capsid proteins are the only visible protein bands in an acceptable prep), assayed for sterility and lack of endotoxin, and then aliquoted and stored at −80° C.

### 2: ABR Measurement

The mouse was anesthetized with a ketamine and Xylacine mixture (60-80 mg/kg+10 mg/kg respectively i.p.) and put on a thermostatic heating pad to keep the body temperature at 38.5° C. Signal generation and ABR acquisition employed Tucker-Davis hardware and BioSig software (Tucker-DavisTechnology system III). The stimuli consisted of tone bursts at 2, 4, 8, 16, 32, 48 and 64 kHz, with a duration of 10 ms and rise/fall of 1 ms (Blackman window). The stimulation rate was of 21.1/sec, and 1000 evoked responses were averaged for each trial. At each frequency, the ABR was tested by starting with 90 dB sound pressure level (SPL) and then decreasing stimulation SPL in 5-10 dB steps until the threshold for detecting a repeatable response was reached. The evoked responses were recorded by sub-dermal electrodes, band-pass filtered between 100-3000 Hz, before amplification. If the evoked response was not detected at the highest sound presentation level (90 dB SPL) at any given frequency, the threshold at this frequency was labeled as 100 dB SPL

### 3: Cytocochleogram

The methods for determining cochlea morphology were similar to those reported by others in the past (Ding et al., 1999b; Ding et al., 2001). The cytocochleogram was determined by the spatial-percentage count of missing hair cells along the cochlea duct. To do this, the mouse was deeply anesthetized with an over-dose of Ketamine, and the cochlea rapidly harvested after the final ABR test. Surrounding soft tissues were removed, and the round window and oval window were both opened. A small hole was made with a needle at the apex of the cochlea for perfusion and staining. The staining solution for succinate dehydrogenase (SDH) histochemistry was freshly prepared by mixing 0.2 M sodium succinate (2.5 ml), phosphate buffered saline (2.5 ml) and nitro-tetranitro blue tetrazolium (nitro-BT, 5 ml). The cochlea was gently perfused through the hole at the cochlea apex and the opened round and oval windows. Following this, the cochlea was immersed in the SDH solution for 45 min at 37° C., and then fixed with 10% formalin for 4 hours. After fixation, the cochlea was decalcified with 5% EDTA solution for 72 hours. The organ of Corti was dissected and surface preparations were made on slides. Cytocochleograms were established using normative data for C57 mice with custom-made software.

### 4: Western Blotting

Western blotting was employed to quantify the both endogenous XIAP and XIAP-Myc in both cochleae and brain tissues. Soft tissue was harvested as much as possible from each cochlea, and a 2 mm3 piece of brain tissue was also taken from the temporal lobe of each mouse. Tissues were homogenized in RIPA buffer (1% Triton X-100, 1% SDS, 8.77% NaCl, 2.42 Tris-HCl base and 5% Deoxycholic acid, pH 8) and then centrifuged at 14,000 g for 10 min at 4° C. Supernatants were transferred to a new 1.5 ml tube. Protein concentrations were estimated using Bio-Rad reagent and a microplate reader (ELx 800 UV, Bio-tek Instrument Inc.). Following this, 20 μg of protein from each sample was transferred into a tube containing RIPA, 2×SDS sample buffer (7.5 μL each) and DTT (15 mg/mL). The sample was stored at −80° C. for later use. The sample was then separated by 10-15% SDS-polyacrylamide gel electrophoresis in running buffer and then transferred to PDVF membrane. The membrane was blocked in blocking solution (containing 1M Tris-HCl 25 ml, 1M NaCl 150 ml and Tween-20 500 μl, 5% non-fat milk powder in 1 Liter) overnight at 4° C. The blots were then probed with a primary antibody directed against the epitope of both endogenous the XIAP and the XIAP-Myc (1:1,500, XIAP Ab mouse, BD Biosciences 610762), in addition to an antibody for β-actin (1:20,000; Sigma A5441), followed by anti-mouse IgG horseradish peroxidase-linked antibody (1:10,000; Vector Laboratories, PI-2000). Band detection was achieved using ECL Plus Kit (GE Health Care) and read with the Storm 840 gel analysis system. The β-actin band was used as internal reference for the level of both endo-XIAP and XIAP-Myc.

The level of XIAP (both endo-XIAP and XIAP-Myc) was calculated as a volume ratio between the XIAP and β-actin. The expression of endogenous XIAP was evaluated against three factors (age: 2 verses 14 months, genotype: TG versus WT, and tissue: cochlea versus brain) in 3 way ANOVA using a=0.05 as significance level. Post Hoc paired tests were performed within the age and tissue factors, because these factors achieved significance on ANOVA testing. The expression of XIAP-Myc was also evaluated against age and tissue type in two-way ANOVA (p<0.05).

### 5: Data Analysis

Hearing was documented in audiograms, which show the ABR thresholds as a function of frequency, and compared between the two groups over time. Two-way ANOVA was performed against the factors of genotype and frequency at each time point when hearing sensitivity was evaluated with ABR. If a significant effect of genotype factor was found, post-Hoc tests will be done to verify at which frequencies, the difference between the two groups would be significant. Hair cell loss was documented in the cochleograms of the surviving 17 mice in the TG group and the 15 mice in the WT group. Loss of both inner hair cells (IHCs) and outer hair cells (OHCs) was counted, and the total loss was compared between the two groups.

### 6: Injection Methods

For animal modeling, the following techniques can be used.

### A. Round Window Method

Adult guinea pigs or rats are anesthetized. A dorsal postauricular incision is made and the bone medial to the tympanic ring is exposed. A hole is drilled exposing the middle ear space medial to the tympanic ring. The round window niche and the bone overhanging the niche are both identified. The bone is scraped away revealing the round window membrane. Vector injections are carried out using a microsyringe. A needle is used to puncture the round window membrane using a micromanipulator while the animals are immobilized to minimize injection trauma. After the injection, the round window is patched with a small piece of muscle tissue or fascia. For each approach, control animals are injected with artificial perilymph to control for damage induced by hydraulic forces. Hearing is tested by ABR audiometry.

For the round window route, it is also possible to use transtympanic injection, and to allow either active or passive uptake of the vector via the round window membrane without puncture. In other methods, the agent may be applied onto an absorbable material such as Gelfoam® that is placed against the round window, and delivers the vector to the round window. In other methods of delivery, an active controlled release pump is used to direct the agent in solution at a predefined rate to the round window area. In other methods of delivery, a passive wick is placed against the round window membrane, and the agent is applied to the lateral end of this wick for delivery by wicking action to the round window membrane.

### B. Semicircular Canal Method

After surgically exposing the temporal bone, the superior semicircular canal is identified. An argon, KTP, CO₂ or other laser, or drilling with a fine drill is used to create an opening in the bone. Injection of the vector and data acquisition is carried out.

### C. Cochleostomy

Animals are prepared as previously described above. The promontory overlying the basal turn of the cochlea was identified. An argon, KTP, CO₂ or other laser or a fine drill is used to create a cochleostomy anterior to the round window, or inferior to the oval window. Injection of vector is then carried out. The cochleostomy is sealed and the animal is allowed to recover.

Additionally, the vector can be injected into the cerebro-spinal fluid (CSF) and monitor the vector's progress via cochlear aqueduct or internal auditory canal into inner ear.

### 7: Vector Delivery and Transfection in Humans

For human subjects, injection may be done via the lateral semicircular canal, which can be accessed either by drilling or by use of a laser, such as for example, a CO₂ laser. In one example, a saline solution of the vector which encodes for X-linked inhibitory protein (XIAP) is injected into the inner ear via a hole prepared as above. Transfection of XIAP causes diminution of the apoptosis and loss of hearing associated with aging and can allow surgical intervention days after the initial transfection, as the transfected hair cells will be producing XIAP. Additionally, the vector can be administered to a human subject by a stapedotomy or stapedectomy or via diffusion of the vector across the round window membrane, including placing the vector onto an absorbable carrier such as gel foam, or non-absorbable carrier in contact with the round window membrane or via an active micro-pump or passive wicking system to the round window.

8. Enzyme Facilitated Cochlear Gene Transfection with AAV

Under appropriate anesthesia, the round window of the guinea pig was surgically exposed and inspected visually under a surgical microscope. Freshly prepared collagenase solution of 2-3 ul was applied to the round window niche with the help of a microinjection pump. The reaction time was 10 minutes. The residual solution was then sucked out and the RWM was washed with saline. The collagenase was used at a concentration of between 50 unit/ml to 200 unit/ml. In one example, the concentration used was 150 unit/ml. In the example provided here, the collagenase was collagenase from Clostridium histolyticum, Type II (available from Sigma). Other proteolytic enzymes such as, for example, papain, trypsin, pepsin, chymotrypsin or elastase could also be used to partially digest the round window membrane. Also, a miniosmotic pump can be used to continuously deliver the MV in the vicinity of the round window membrane so as to saturate the membrane with the AAV vector.

To evaluate the damage of the digesting solution on RWW, the animal was killed immediately after the digestion and the cochleae were taken out and fixed with standard protocol for electronic microscopy. To evaluate if the damaged RWM heals spontaneously, some other animals were allowed to survive for 3 weeks before the RWMs were taken for examination.

For inner ear gene transfection, a piece of gelfoam (1-2 mm³) was placed gently on RWM. 5 ul AAV solution was applied to the gelfoam. The cochlea was taken out for cochlea immunostaining against GFP 2-4 weeks after the surgical application of AAV vectors.

ABR was tested in animals underwent gene transfection before and just before the animal was killed for morphology.

This procedure is also applicable in the use of the adeno-associated viral expression vectors, or mutants thereof, which encode XIAP so as to locally transfect the cells.

### 9. Surgical Procedure for AAV Injection

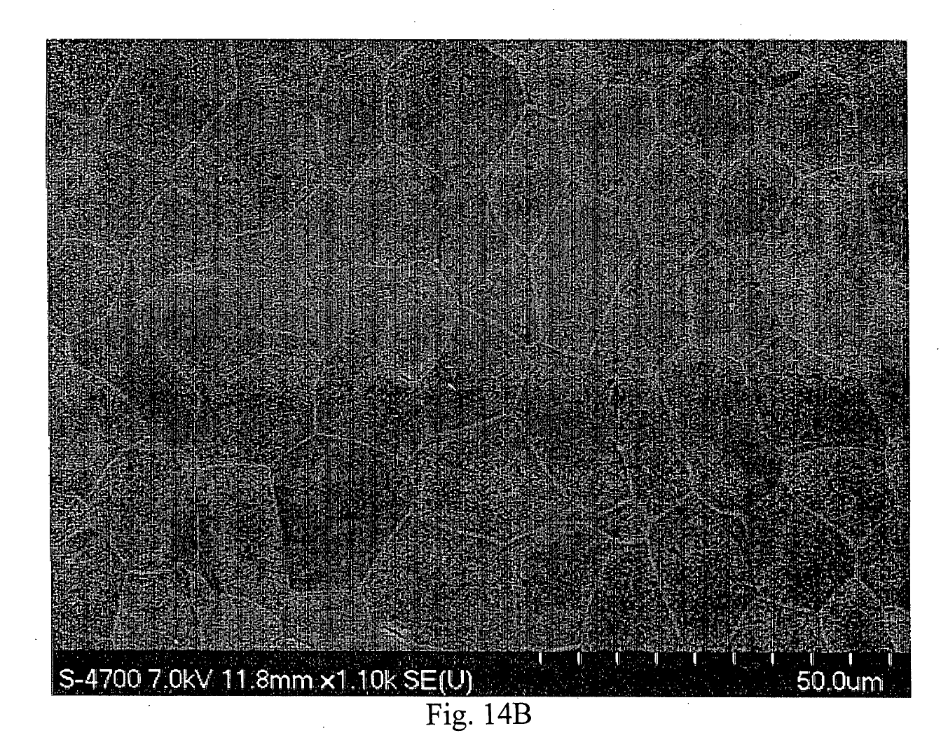

Patients identified as suffering from aging related hearing loss would be eligible for XIAP gene therapy. The cochlea would be accessed via the round window membrane (RWM) that has been partially permeabilized using a proteolytic enzyme such as collagenase. The RWM in turn would be accessed via a small incision in the tympanic membrane. Specifically, a myringotomy (surgical procedure in which a small incision is created in the eardrum) will be performed to access the round window membrane (RWM) under local anesthetic as an out patient procedure in adults. Collagenase will be applied to the round window membrane (10 ul) at a concentration of 150 unit/mL for 10 min. After this time, the collagenase solution in contact with the round window membrane is aspirated and the membrane washed with sterile solution to remove any residual enzyme. Our analysis of the round window membrane by transmission electron microscopy has shown that the damage produced by collagenase treatment is temporary and by 3 weeks the round window membrane (RWM) has nearly completely healed (FIG. 14). 20 ul AAV solution (tyrosine mutant AAV serotypes 2-8, titer 1-5E13) will be applied to gelfoam. For transfection of sensory hair cells and related conduction cells, the AAV containing gelfoam (5 mm3) will be placed gently on the RWM. The gelfoam will be allowed to remain in contact with the RWM where it is slowly broken down and absorbed into the surrounding tissue in a non-injurious manner. This procedure is also applicable in the use of the adeno-associated viral expression vectors, or mutants thereof, which encode XIAP so as to locally transfect the cells. Other methods of delivery include active micropumps, and passive wicking systems. The Gelfoam may be covered with a layer of fascia to prevent any perilymphatic fistulae after the treatment.

### 10. Delivery of Candidate Genes Encoded by Tyrosine Mutant AAVs Across the Round Window Membrane:

Under appropriate anesthesia, the round window of the guinea pig is surgically exposed and inspected visually under a surgical microscope. A freshly prepared solution composed of an enzyme, an encapsulated small molecule, a biocompatible detergent or other reagent designed to enhance the permeability of the round window membrane described herein, is applied to the round window niche at a flow rate of 5 ul/ml with the help of a microinjection pump. The reaction time is 10-40 minutes. These treatments serve to increase the permeability of the RWM. The residual solution is then sucked out and the RWM washed with saline (10 ul). A biodegradable matrix containing a tyrosine mutant AAV (1×10⁸⁻¹³ particles) encoding an ototoprotective and/or ototoregenerative gene(s) is applied to the RWM. This procedure will increase the expression of genes listed in 1-5 in sensory hair cells and spiral ganglion of the cochlea and vestibular system.

Improved penetration of tyrosine mutant AAV into the inner ear by the passage of air over the RWM causing a mild drying effect (Milkulec et al. Otol Neurotol 2008 Oct. 29(7): 1020-1026). A #3 French suction is placed near the round window niche to avoid direct trauma to the RW membrane, and suction applied for a period of 2 minutes. A biodegradable matrix containing a tyrosine mutant AAV will then be applied to the RWM as described in 1.

Under appropriate anesthesia, the round window of the guinea pig is surgically exposed and inspected visually under a surgical microscope. A freshly prepared solution composed of an enzyme, an encapsulated small molecule, a biocompatible detergent or other reagent designed to enhance the permeability of the round window membrane described herein, is applied to the round window niche at a flow rate of 5 ul/ml with the help of a microinjection pump. The reaction time is 10-40 minutes. These treatments serve to increase the permeability of the RWM. The RWM is then washed with saline (10 ul). A biodegradable matrix saturated with liposomes is then applied to the RWM. This procedure will increase the delivery of microencapsulated compound to the RWM and reduce hearing triggered by acoustic trauma, ototoxic drugs such as cisplatin or aging related hearing loss.

### 11. OHC Transfection Using Mutant AAV Vector.

The transfection is through intact round window membrane (RWM). Under appropriate anesthesia, a cut of 2 cm is made posterior to the earlap and the soft tissue is distracted to expose the mastoid. The round window of the guinea pig is exposed by drilling a small hole of 2-3 mm in diameter on the mastoid. Solution of collagenase II is freshly prepared at concentration of 150 units/ml. With the help of a microinjection pump, a total of 10 μl enzyme solution is applied to the round window niche at a flow rate of 10 nl/sec. After reaction time of 10 minutes, residual enzyme solution is sucked out first using suction pump and then use gelfoam. A piece of gelfoam (2-3 mm³) is placed upon RWM and then 5 μl AAV-GFP is applied to the gelfoam. The hole of mastoid is covered by suturing the soft tissue. Hearing status is evaluated using audiotory brainstem response (ABR). The surgery does not cause significant hearing loss.

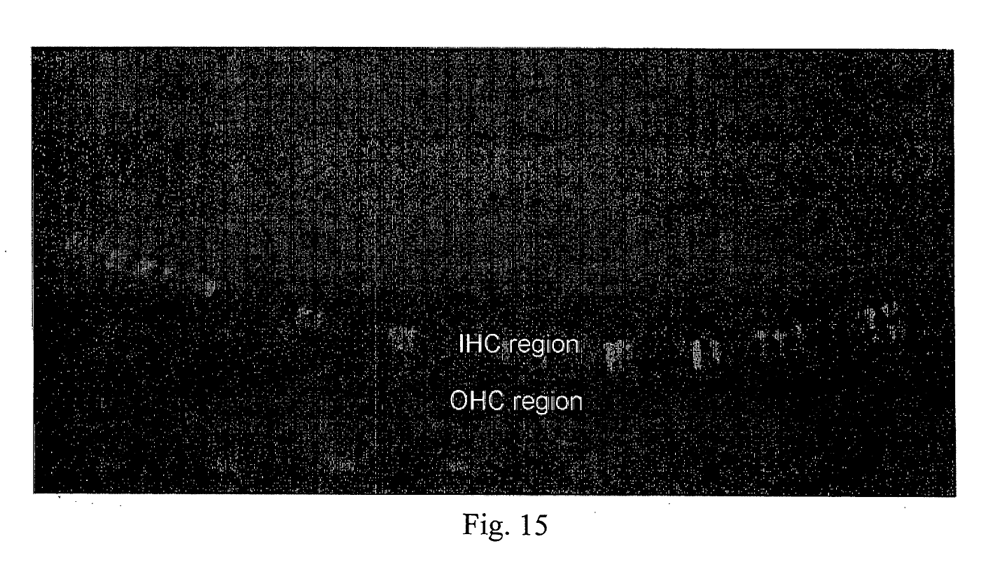

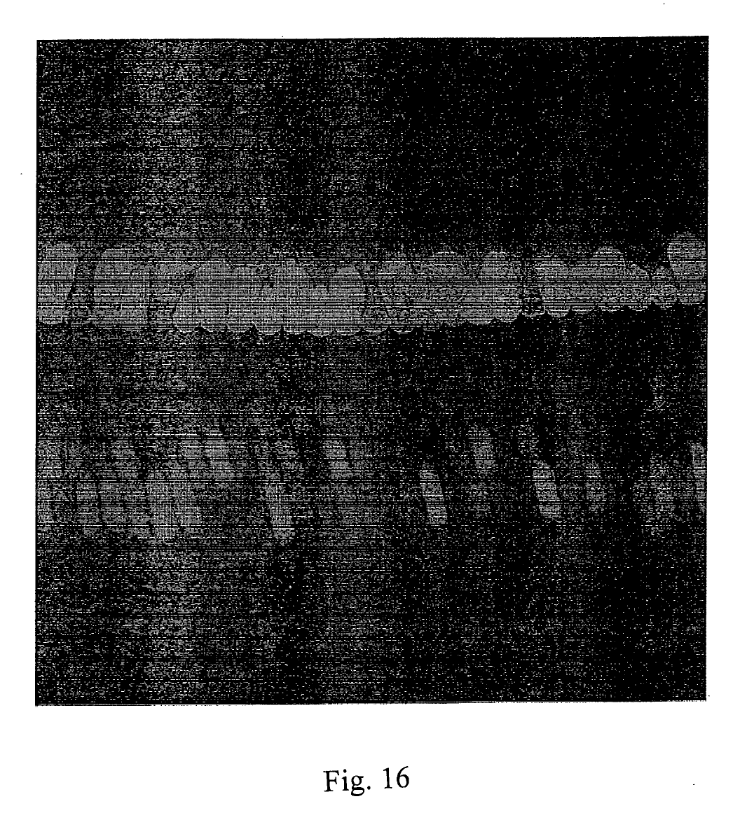

The transfection is evaluated 2 weeks after the surgery. Briefly, the cochlea is harvested and basilar membrane (with the organ of Corti) is dissected after fixation. Immunohistochemistry staining against GFP is performed and the tissue is spread on cover slid in the form of surface preparation. The sample is then observed using fluorescent microscope. GFP positive hair cells are counted along the length of the cochlear duct. The results are shown in FIGS. 15 and 16. Both images are taken from corresponding basal regions of cochlea.

12. Use of Ultrasound as a Delivery Method for Gene Therapeutics into the Cochlea.

To transport viruses or other gene therapies into the cochlea without sacrificing the integrity of the round window membrane, ultrasound is contemplated as a method for enhancing the permeability of the round window membrane. Recently, it has been shown that microbubble and nanoparticle contrast agents have the ability to increase the effectiveness of high-intensity focused ultrasound (HIFU) therapy by ablating tissues through the disruption of cavitation bubbles (Ken-Ichi Kawabata, Rei Asami, Takashi Azuma, Hideki Yoshikawa and Shin-Ichiro Umemura, “High Intensity Focused Ultrasound (HIFU) Therapy with Nano Droplets and Microbubbles,” Ultrasound in Medicine and Bio. Vol. 35, num. 8S, 2009). Ablating tissues through cavitation bubbles is an attractive alternative to the conventional thermal ablation achieved with HIFU therapy since there is no destruction of neighboring tissues that surround the bubble cloud. It has also been recently suggested that the permeability of vascular walls and other low permeability tissues such as the neural retina may be greatly increased by disrupting nanoparticles near the membrane boundary (U.S. Pat. No. 6,165,440, “Radiation and nanoparticles for enhancement of drug delivery in solid tumors,” Dec. 6 2000; and Liesbeth Peeters, Ine Lentacker, Roosmarijn E. Vandenbroucke, Bart Lucas, Joseph Demeester, Niek N. Sanders, and Stefaan C. De Smedt, “Can Ultrasound Solve the Transport Barrier of the Neural Retina?,” Pharmaceutical Research, Vol. 25, No. 11, November 2008). This would suggest that the permeability of the round window membrane of the cochlea may also be increased through the disruption nanoparticles near the membrane boundary.

Nanoparticles have the added advantage that they may be loaded with antibodies, and/or other drugs/genes/viruses. Therefore, if a nanoparticle can be developed such that it is loaded with a therapeutic for the inner-ear, it is reasonable to assume that upon disruption of the nanparticles in the vicinity of the round window membrane, the resulting perforation of the round window and subsequent release of the therapeutic agent will greatly increase the payload inside the cochlea. If the nanoparticles are easily disrupted with pulsed ultrasound waves (i.e. perfluorocarbon Droplets (PFC)), then the particles have the added advantage of being “pushed” toward the round window by the radiation forces inherent to the ultrasound waves. In addition, it has also recently been shown that nanoparticles such as PFC droplets can be electrically “charged”. This could potentially increase the permeability of the round window membrane as well.

### 13. Use of Ultrasound and Nanoparticles to Increase RWM Permeability Enabling Better Penetration of Tyrosine Mutant AAV

Under appropriate anesthesia, the round window of the guinea pig is surgically exposed and inspected visually under a surgical microscope. A freshly prepared saline solution composed of nanoparticles (for example, perfluorocarbon, polystrene) coated with Pluronic F-127 (Sigma-Aldrich) at a concentration of 1×10¹⁴ particles/μl are applied to the round window niche (10 nl). The reaction time is 60-120 minutes. A high frequency sonic probe (2 mm diameter) is then placed against the RWM. Ultrasound is then applied at a frequency of 1-50 MHz and an intensity of 0.5-10 W/cm2 for 30-120 seconds to create peak pressures ranging from 1-10 Mpa at the surface of the RWM. This treatment increases the permeability of the RWM by causing cavitation and bubble disruption. Next, a biodegradable matrix containing a tyrosine mutant AAV (1×10⁸-12 particles) encoding an ototoprotective and/or regenerative gene(s) is applied to the RWM. This procedure will increase the expression of genes listed in 1-5 in sensory hair cells and spiral ganglion of the cochlea and vestibular system.

### 14. Use of High Osmolarity to Increase RWM Permeability Enabling Better Penetration of Tyrosine Mutant AAV

Under appropriate anesthesia, the round window of the guinea pig is surgically exposed and inspected visually under a surgical microscope. A freshly prepared high osmolarity solution (620 mOsm) composed of (mM) NaCl (127.5), KCl (3.5), NaHCO₃ (25), CaCl₂ (1.3), MgCl₂ (1.2), NaH₂PO₄ (0.75), Glucose (11), mannitol (320) is used to irrigate the RWM for 40 minutes at a rate of 5 μl/min. Next, a biodegradable matrix containing a tyrosine mutant AAV (1×108-12 particles) encoding an ototoprotective and/or ototregenerative gene(s) is applied to the RWM. This procedure will increase the expression of genes listed in 1-5 in sensory hair cells and spiral ganglion of the cochlea and vestibular system.

### 15. Protective Effect of X-Linked Inhibitor of Apoptosis Protein Against Noise Induced Cochlear Lesion in C57 Mice

### Subjects and Experiment Overview

Transgenic founders were generated as previously described (Wang et al., 2008). Briefly, a linearized plasmid construct consisting of the Ubiquitin C promoter, 6 repeats of the 9E10 myc epitope tag fused to the amino terminus of the human XIAP coding region, and a polyadenylation signal from SV40 was microinjected into the male pronucleus of C57BL/6J X C3H F1 zygotes. C57BL/6J X C3H F1 offspring were backcrossed over 15 generations against the wild-type (WT) C57BL/6J mice to obtain ubXIAP transgenic animals on a pure C57BL/6J genetic background (TG mice). To obtain wild-type (WT) littermates as controls, ubXIAP animals were crossed with WT C57BL/6J mice (F10) to obtain TG mice on a C57BL/6J background. Transgenic status within the colony was determined by PCR targeting the 6-myc tag. All transgenic mice and their wild-type littermates used in this experiment were bred in the Facility for Animal Care, Dalhousie University. Mice were 2-4 months of age during the experiment. The two experimental groups were matched for age and gender.

The impact of noise on hearing function and cochlear morphology was examined in two groups of mice, one composed of 15 ubXIAP (TG) animals and the other 15 WT littermates. Frequency-specific auditory brainstem responses (ABR) were recorded as an index of hearing status before and at different time points up to one month after the noise exposure. Those animals with abnormal hearing, verified by a baseline test, were excluded. After the final ABR test, all mice were sacrificed under deep anaesthesia and their cochleae were harvested. From the first 5 mice in each group, both ears were used for cytocochleograms in surface preparation for hair cell (HC) loss. In the later 10 mice in each group, one ear from each mouse was used for cytocochleograms and the other for evaluating the damage to spiral ganglion neurons (SGNs) and nerve fibers by cross-sectioning. In addition, 6 cochleae from 6 mice experiencing no noise exposure were used to establish control norms for the axon fibers and SGN counts. To evaluate the impact of noise exposure on the level of XIAPs (both endogenous and that from the transgene), an additional 40 animals (20 WT and 20 TG) were used for Western blot analysis. In each genotype, 10 cochleae from 5 mice were used as no-noise controls and the other 30 cochleae from 15 mice were harvested 24 hours after exposure to the noise.

### ABR Recording

For ABR recordings, mice were anaesthetized with Ketamine+Xylazine (80-100 mg/kg+10 mg/kg respectively i.p.). An additional one fourth of the initial dose was administered as needed. The mice were placed on a thermostatic heating pad to keep the body temperature at 38.5° C. during the procedure. Tucker-Davis hardware (Tucker-Davis Technology system III) and BioSig software were used for the signal generation and acquisition of ABR in response to tone bursts of 2-64 kHz in octave steps, with a duration of 10 ms and rise/fall of 1 ms. The signal was delivered through an broadband electrostatic speaker (ES1 from TDT) which was placed 10 cm in front of the animal's head in a sound-proof booth. At each frequency, the signal was presented from 90 dB SPL down to 10 dB SPL in 5-10-dB steps. The sound level was calibrated using a ¼-inch B&K condenser microphone (Mode 4939) that was placed at the position that would be occupied by the head of the animal. The output of the microphone was examined using SigCal software from TDT.

Sub-dermal electrodes were used for ABR recording, with the active recording electrode on the vertex of the skull and the reference and ground behind each ear. The responses were band-pass filtered between 100-3000 Hz, amplified and averaged over 1000 times with a repetition rate of 21.1/s. The threshold was judged as the lowest SPL at which a repeatable response was visible. If no waveform was identified at the highest presentation level (90 dB SPL) for a particular frequency, the threshold was then documented as 100 dB SPL.

### Noise Exposure

Mice were exposed to noise in a sound proof booth, unanesthetized and unrestrained, numbering five animals per each cage. The noise consisted of two pure tones at 1 and 6 kHz respectively and with equal intensity to make the total level of 125 dB SPL.

### Cytocochleogram

The methods for determining cochlear morphology are similar to those reported by others (Ding et al., 2001; Ding et al., 1999). The cytocochleogram was determined by the spatial-percentage count of missing HCs along the cochlear duct. After the final ABR test, the mice were deeply anaesthetized with an over-dose of ketamine, and the cochleae rapidly harvested. Surrounding soft tissues were removed and the round window and oval window were both opened. A small hole was made with a needle at the apex of the cochlea for perfusion and staining. The staining solution for succinate dehydrogenase (SDH) histochemistry was freshly prepared by mixing 0.2 mol sodium succinate (2.5 ml), phosphate buffered saline (2.5 ml) and nitro-tetranitro blue tetrazolium (nitro-BT; 5 ml). The cochlea was gently perfused through the hole at the cochlear apex and the opened round and oval windows. Following this, the cochlea was immersed in the SDH solution for 45 min at 37° C., and then fixed with 10% formalin for 4 hours. After fixation, the cochlea was decalcified with 5% EDTA solution for 3 days. The organ of Corti was dissected and surface preparations were made on slides. Cytocochleograms were established against the norm for C57 mice using custom software (as previously reported by Wang, Ding & Salvi, 2003). HC loss was then measured in the prepared sections of the OC.

### Cochlear Cross-Section

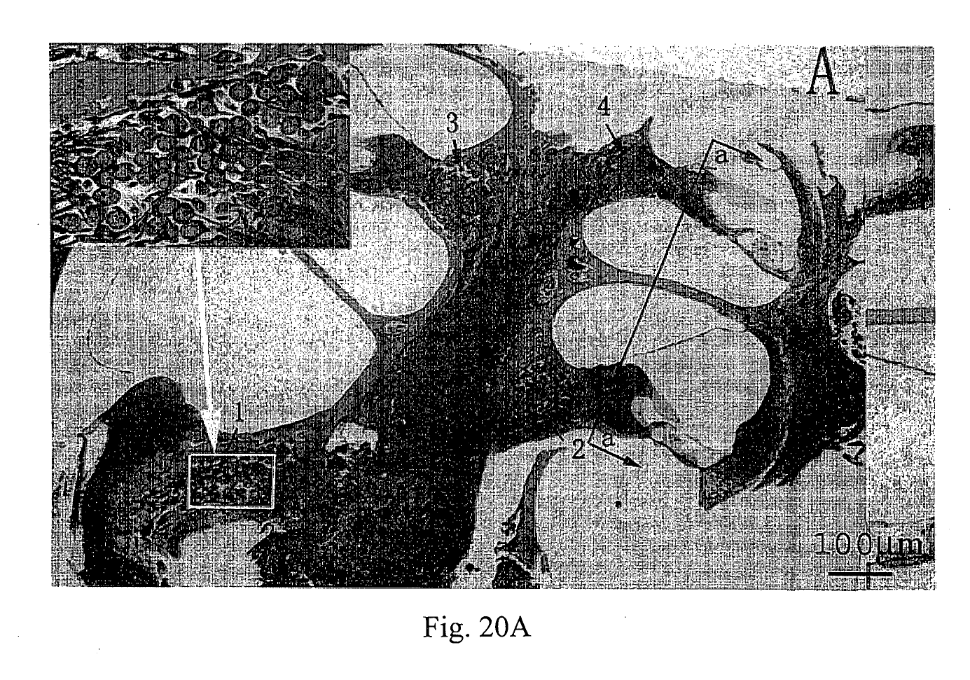

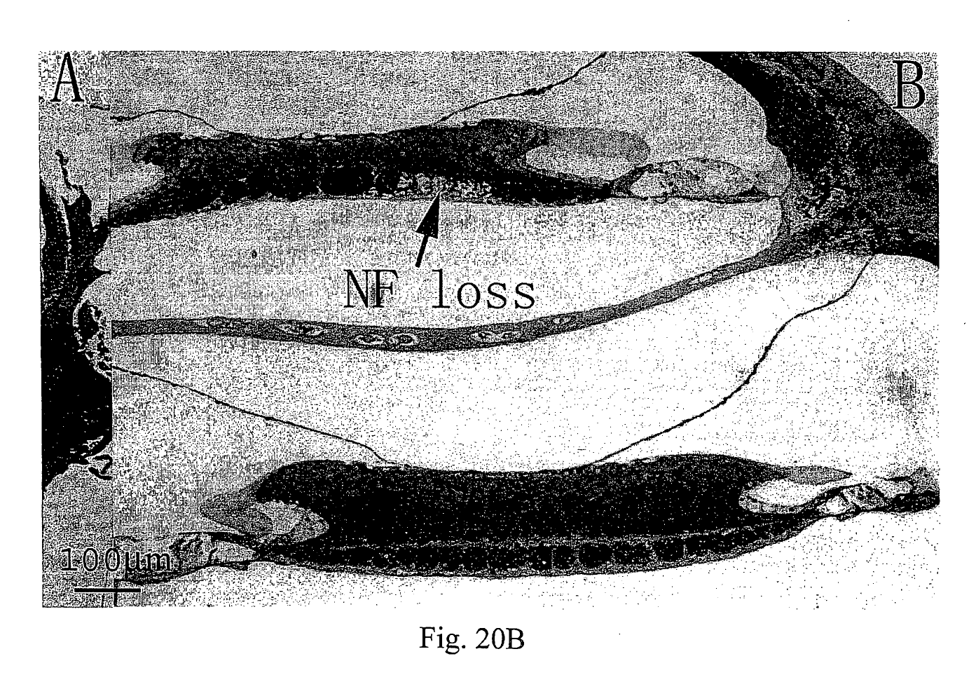

Histological preparations for the examination of the SGN lesion were similar to that of the cytocochleogram preparation with slight modifications. Briefly, the cochlea was perfused with 2% glutaraldehyde in PBS buffer for fixation. After the perfusion, the cochlea was immersed in the fixative for 6 hours at 4° C. followed by decalcification in 5% EDTA for 3 days. The cochlea was further fixed in 1% osmium acid for one hour at room temperature and then dehydrated in grade ethanol. Then, the sample was infiltrated with a 1:1 volume ratio of propylene oxide+Epon at room temperature overnight and then transferred into 100% Epon for 4 hours. Next, the sample was immersed in 100% Epon in container to be hardened in oven at 60° C. for more than 12 hours. Semi-thin cross-sections of 1.5-2 μm were made along the axes of modiolus with microtome equipment and were transferred to a glass slide, stained with 1% Toluidine blue for 1 minute, and then examined under a light microscope. The number of SGN cell bodies was counted in the Rosenthal canal at four locations corresponding to turns along the cochlear duct (two positions each for the basal and apical turns of the cochlea; four positions in total, see FIG. 20). At each location, 10 sections were taken to cover a distance over 0.4 mm and the SGN cell body counts averaged from the 10. The numbers of auditory nerve fibers (dendrites from the SGN) were also counted in the sections crossing the Hebanular perforate. For this measure, dendrites in Hebanular perforates immediately proximal to the Rosenthal canal were quantified. The number of nerve fibers was averaged from 10 Hebanular perforates in each turn for each ear. The counting of nerve fibers (dendrites originating from SGNs) and SGN cell bodies was carried out using the cell counter function of ImageJ software (NIH, USA).

### Western Blotting

Western blotting analysis was used to quantify the levels of both endogenous XIAP (wild-type, WT) and human XIAP derived from the ubXIAP transgene (TG mice) in the cochlea. Electrophoresis was performed in the Western blot apparatus to separate the proteins in a membrane according to their mass. For this electrophoresis analysis, proteins were extracted from the soft tissue of the cochlea and a piece of brain, measuring 2 mm³, from every animal using a standard protocol. Tissues were homogenized in RIPA buffer (1% Triton X-100, 1% SDS, 8.77% NaCl, 2.42 Tris-HCL base and 5% Deoxycholic acid, pH8) and then centrifuged at 14,000 g for 10 min at 4° C. Supernatants were transferred to a new 1.5-ml tube. Protein concentrations were estimated using Bio-Rad reagent and a microplate reader (Elx 800 UV, Bio-tek Instrument Inc.). Next, 20 μg of protein from each sample was transferred into a tube containing RIPA, 2×SDS sample buffer (7.5 μL each), and DTT (15 mg/mL). The sample was then separated by 10-15% SDS-polyacrylamide gel electrophoresis in running buffer then transferred to PDVF membrane. The membrane was blocked in a blocking solution (containing 1 mol Tris-HCL 25 ml, 1 mol NaCl 150 ml and Tween-20 500 μl, 5% non-fat milk powder in 1 L) overnight at 4° C. After the proteins were separated, they are transferred to Whatman paper and incubated in a solution containing the primary antibody that recognizes a region conserved in both mouse XIAP (WT) and human XIAP (1:1,500, XIAP antibody mouse, from BD Biosciences). After adequate rinsing, the membrane was incubated with the secondary antibody (anti-mouse IgG horseradish peroxidase-linked antibody, 1:10,000, from Vector Laboratories, PI-2000) for a minimum of one hour. This secondary antibody will cause the targeted bands to be coloured in the membrane. The XIAPs (human and mouse) were then quantified by analyzing the bands on the membrane using a method of density analysis in 6 WT and 6 TG mice.

### Data Analyses

The primary focus of the data analysis was to test whether XIAP over-expression resulted in reduced noise-induced cochlear cell death (namely, HCs and SGNs), and its effect in preventing NIHL. For this purpose, we first evaluated the shift of hearing thresholds measured by ABR between the two different groups of WT and TG by an analysis of covariance (ANCOVA). The genotype group was a fixed factor and frequency a continuous covariate. When a result was statistically significant at the level of α<0.05 or better, post hoc tests (paired t-test with the Bonferroni correction) were used to further evaluate the significance. ABR testing was used to verify whether XIAP over-expression reduces hearing loss due to intense noise exposure over the long term by examining thresholds at both 1 and 4 weeks post noise exposure.

In histological examinations, the evaluation of HC loss was conducted by cytocochleograms. We did not focus on the pattern of cell death but performed a spatial percentage HC count. The Student t-test was used to evaluate the significance of differences of total HC loss between the WT and TG groups. SGN and nerve fiber counts were conducted and compared across three groups of samples (no-noise control and noise-damaged cochleae from both TG and WT mice) using a one-way analysis of variance (ANOVA). When a result was statistically significant at the level of α<0.05 or better, post hoc tests (paired t-test) were used to further evaluate the significance.

The information gained from Western Blotting analysis gave additional insights into the protective effects of XIAP over-expression by the examination of the density of the protein bands. The first series of analyses determined if there was any difference in the level of endogenous XIAP in the noise-exposed WT and TG groups versus the controls. A within-subject ANOVA was performed to answer the following questions: (1) Does endo-XIAP differ between WT and TG groups in none-noise-treated animal? (2) Does noise exposure induce increases in endo-XIAP? These analyses provided insight as to whether or not the transferred XIAP gene interfered with expression of the endo-XIAP gene.

Data was qualified as significant at the level of α<0.05 and presented in the form of the mean±SEM.

### 16. Cochlear Staining

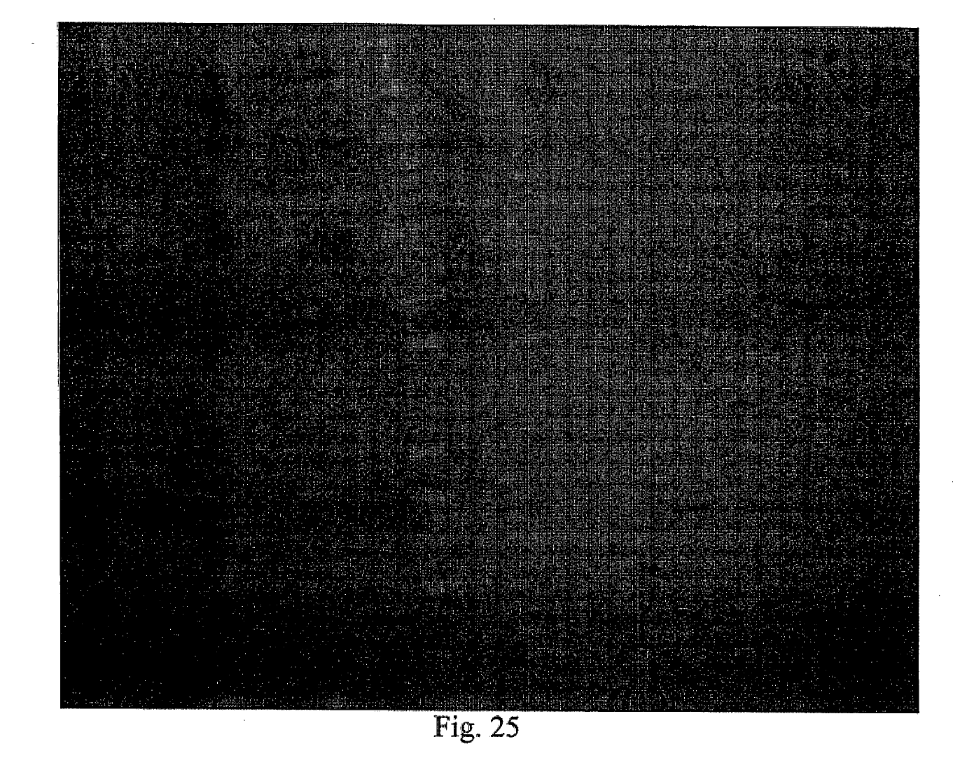

A post-auricular approach was used to expose the tympanic bony bulla. After local analgesia with lidocaine, a retro-auricular incision was made to expose the mastoid. A hole of 2-3 mm in diameter was drilled on the mastoid to expose the RW niche and the bony wall of the cochlea immediately inferior to the RW niche. A micropump (Micro4™ from WPI, FL USA) was used to drive a microsyringe for the application of solution. For the digestive treatment, the solution of collagenase (either type I or 11, C0130 or C6885 respectively from Sigma) was freshly prepared in distilled water just before the surgery. The duration for the digestive treatment varied from 5 to 10 minutes and the quantity of the digestive solution 5 to 10 μl. In any case, the solution was first injected at a high speed and the injection was observed under surgical microscope to ensure that the RW niche was filled with the solution. Then, the injection was continued at a low speed so that the desired volume was given out during the desired period of time. Then the residual solution inside the RW niche and in the surrounding area was removed using suction by paper tips. A piece of Gelfoam (5-10 mm³) was inserted gently to make contact with RW. For the safety observation, the surgery was stopped here and the opening of the bulla and the skin incision were closed by sutures. For gene transfection, 5-10 μl of viral vectors were applied into the Gelfoam before the suture. For the transfection through cochleostomy, a small hole of 0.3 mm in diameter was drilled through the bony shell of basal turn of the cochlea into the scala tympani. rAAV2 or 8 vectors were obtained from Vector Gene Technology Company Limited of China (rAAV2-EGFP vector promoted by hCMV) or supplied as a collaboration of research by Dr. Williams W. Hauswirth's laboratory of Retina Gene Therapy Group, University of Florida USA (rAAV8-GFP mut733). See FIG. 25, which illustrate the images of XIAP staining from a cochlea receiving AAV8-mut-XIAP-6myc. The immunostaining is made against the 6myc tag in the vector. The image show IHC transfection. The location of the sample is from the cochlea: basalturn, 2nd and 3rd turn are from basal, high-frequency region to low frequency region.

### 17. Methods for Transfection Surgery

The subjects were anesthetized in a similar method to that described above for ABR testing. The animal's head was fixed with a stereotaxic restraint, and surgery performed under sterile conditions. A post-auricular approach was used to expose the tympanic bony bulla. After local analgesia with lidocaine, a retro-auricular incision was made to expose the mastoid. A hole of 2-3 mm in diameter was drilled into the mastoid to expose the RW niche and the bony wall of the cochlea immediately inferior to the RW niche. A micropump (Micro4™ from WPI, FL USA) was used to drive a microsyringe to apply the solution. For the digestive treatment arm, a solution of collagenase (either type I or II, C0130 or C6885 respectively from Sigma) was freshly prepared in distilled water just before the surgery. The duration of exposure for the digestive treatment varied from 5 to 10 minutes and the quantity of the digestive solution from 5 to 10 μl. The solution was first injected at a high rate and observed under a surgical microscope to ensure that the RW niche was filled with the solution. The injection was then continued at a low rate to ensure the desired volume was applied during the desired time period. Following this, residual solution inside the RW niche and in the surrounding area was removed using fine paper wicking. A piece of Gelfoam (5-10 mm³) was then inserted gently to make contact with RW. In those animals enrolled in the safety evaluation arm, surgery stopped here and the bulla opening and skin incision were closed. For the gene transfection arm, 5-10 μl of viral vector was applied onto the Gelfoam before closing. In the cochleostomy arm, a small hole of 0.3 mm in diameter was drilled through the bony shell of basal turn of the cochlea into the scala tympani. rAAV2 or rAAV8 vectors were obtained from Vector Gene Technology Company Limited of China (rAAV2-EGFP vector promoted by hCMV) or supplied as a collaboration with Dr. Williams W. Hauswirth's laboratory in the Retina Gene Therapy Group, University of Florida USA (rAAV8-GFP mut733).

### 18. Tissue Preparation for Morphology and Immunostaining

Under deep anesthesia with an overdose of pentobarbital (100 mg/kg), the animal was decapitated and the temporal bone was rapidly removed and the cochlea extracted. To examine the RWM, the cochlea was immersed into a fixative of 2.5% glutaraldehyde overnight at 4° C., and post fixed in 2% OsO4 for 2 hours. The attachment of RWM to the bone ring of the niche was cut in a half circle to reduce the tension in the sample before dehydration with a graded series of ethanol concentrations, concluding with 100%). For the SEM observations, the RWM samples were critical-point dried with liquid CO2, and sputter-coated with gold. Photographic images were taken with a Hitachi S-4700 SEM system. For the TEM observations, the RWM specimens were dehydrated in 100% ethanol, and then embedded in Epon 812, and then 70-nm-thick sections were cut and examined under the TEM microscope (JEM-1230, Japan).

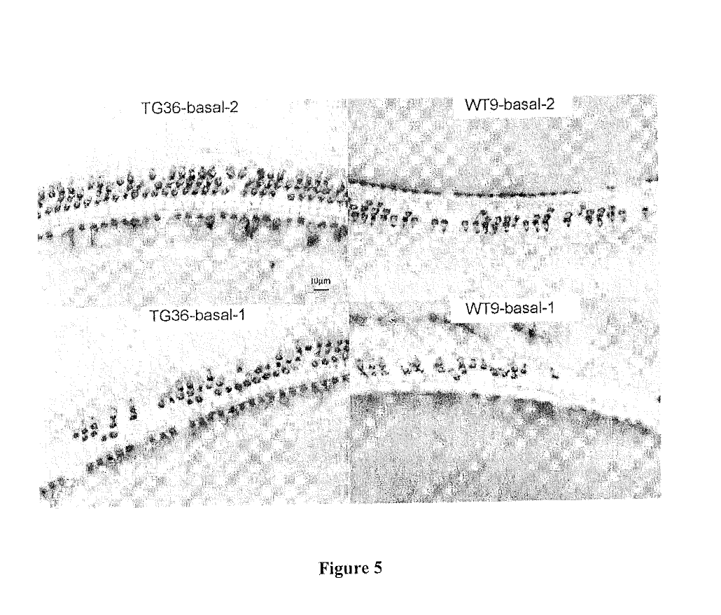

To determine gene expression of green fluorescent protein (GFP, the marker gene carried by the vector), the apex of the cochlea and the round window were punctured and then the cochlea was perfused through the apical hole with fluid exit through the round window with a fixative of 2.5% paraformaldehyde and immersed in the fixative over night at 4° C. The basilar membrane and the organs of Corti were then dissected under a microscope. The remaining portion of the cochlea containing the modiolus was decalcified for 4 days or longer as needed in 0.1 M EDTA, and immersed in 15% and 30% sucrose over night at 4° C., then embedded in OCT for 2 hours. To examine the morphology and transfection of SGNs, 8-wm-thick sections were cut parallel to the midline of the modiolus to visualize Rosenthal's canal. The samples taken from the cochleae that were transfected using the viral vectors (rAAV-GFP) supplied from the Retina Gene Therapy Group (Florida University, USA) were first treated with 0.25% triton X-100 for 1 hr, and then blocked by 10% goat serum for 1 hr, followed by primary antibody against GFP (Polyclonal from rabbit, NB600-308, Novus Biologicals, USA) at a concentration of 1:200 overnight at 4° C. After washing three times in 1×PBS, the samples was treated with the 2^{nd} antibody (goat anti rabbit, 70243-Cy™2 from Jackson ImmunoResearch Laboratories Inc, PA, USL) at 1:200 for 2 hrs at 4° C. and then washed again 3 times in the PBS in a dark room. To distinguish transfection of hair cells versus supporting cells, some samples were double stained with phalloidin-TRITC (P1951, Sigma, USA) at concentration of 50 μg/ml for 10 min at room temperature. This was followed by a final three washes in PBS. After all staining procedures, samples were mounted in glycerin on glass slides and were then ready for examination. In the cochleae that were transfected with rAAV2-EGFP, the signal from EGFP was directly visible without immunostaining. Samples were observed under fluorescent microscopy (Axioplan2, Zeiss) or confocal microscopy (Laser Scanning, ZEISS LSM510 META). Only cells with green emissions clearly standing out from the background were counted as EGFP positive (FIG. 5). The transfection cochleogram for HCs was then established by counting the EGFP positive cells with the microscope focusing on the level of HCs in the organ of Corti.

### 19. Treatment for Patient Undergoing Cisplatinum Treatment or for Ushers Syndrome Treatment

The treatment regimens in a patient undergoing Cisplatininum treatment or for Usher's syndrome would be essentially identical. The patient is placed supine, both ears will be treated. Firstly, the head is turned away from the clinician, and a speculum used to visualize the eardrum under a surgical microscope. The eardrum is anesthetized by any common method (e.g. EMLA® cream, or other local anesthetic creams, local anesthetic into the ear canal, or direct phenol application).

Using aseptic technique, the posterior inferior part of the tympanic membrane is slit in a linear method to expose the round window niche. Mucosal folds are picked away to expose the round window membrane area. The collagenase digestive solution is then placed on a small piece of porous gelatin sponge, such as Gelfoam®, or other similar carrier, such as hyaluronic acid sponge (e.g. Merogel®), and placed in contact with the round window membrane for 5-10 mins to partially digest this membrane.

After this time, the sponge is removed, and any remaining liquid is suctioned out of the round window niche. Following this, a fresh piece of sponge with the viral vectors is placed in contact with the round window membrane. This will be left in place, and is absorbed. The edges of the incision in the eardrum (myringotomy) are approximated, and if wanted, a piece of Gelfoam® or similar is used to stabilize them.

The speculum is then removed, and the procedure is terminated on one ear, and a similar procedure is repeated in the contralateral ear.

### Results

I. Hearing Loss Progress with Age

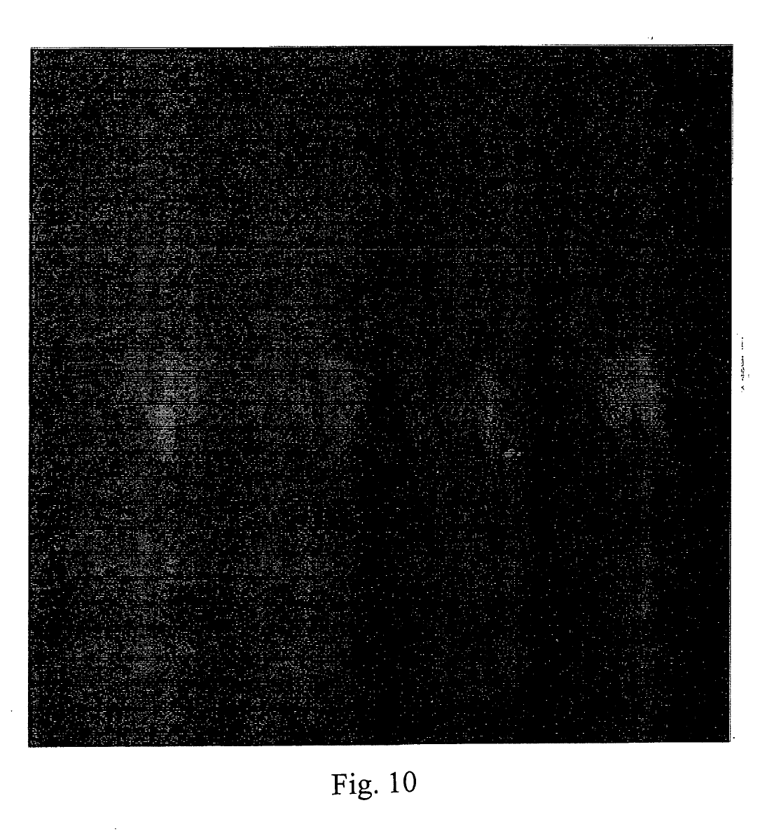

In general, C57BL/6 mice rapidly developed hearing loss starting at very early age (e.g., 2 months). In comparison to WT littermates, TG mice displayed better ABR thresholds at 2 months of age. At this age, hearing loss predominately affected the high frequency regions (FIG. 1A). The differences in averaged thresholds were found to be larger than 5 dB only at the two highest frequencies (48 and 64 kHz) tested. The thresholds were 76.19 and 83.04 dB SPL at 48 and 64 kHz for WT mice, 60.41 and 75.93 dB SPL for TG mice at these two frequencies. A two-way ANOVA identified a significant effect of genotype. The difference between the groups was found to be significant at 48 kHz (Mann-Whitney Rank Sum Test, p<0.05, as indicated by the asterisk in FIG. 1A) but not at 64 kHz. The development of hearing loss was found to be slower in the TG group. This was demonstrated in two different ways in FIG. 1. FIG. 1B shows the ABR audiograms at the age of six months. A significant difference was found in favor of the TG group at frequencies of 8 kHz and above indicating a slower development of high-frequency hearing loss in the TG group. FIGS. 1C and D show the changes of ABR thresholds from 2 months to 6 months in the two groups. In the TG group, the ABR thresholds generally remain unchanged at frequencies below 16 kHz from the values at 2 months of age (FIG. 10). In the WT group, however, the threshold elevation was found to be larger than 5 dB at all frequencies and the change was statistically significant at the frequencies indicated by asterisks (FIG. 10).

In the later stages of the experiment, the threshold differences between the two groups were greater in the high frequency regions but less in the low frequency regions. FIG. 2 shows the ABR threshold changes observed at 10, 12 and 14 months. At 10 months, TG mice showed superior thresholds across all the frequencies tested. Comparison of the data in A, B and C reveal that TG animals retain superior ABR thresholds in the high-frequency region (16, 32, 48 and 64 kHz) relative to WT mice; however, within the low frequency region (2, 4 and 8 kHz) the differences become smaller with further aging, and disappear by 14 months.

This difference between the TG and WT mice is again illustrated in FIG. 3, which presents the data in two different ways. In 3A, the ABR thresholds were averaged across two separated frequency segments (2, 4 and 8 kHz as the low-frequency (LF) region (solid lines), and 16, 32, 48 and 64 kHz as the high-frequency (HF) region (dashed lines)). It appears that, the between-group difference in the high-frequency region is already apparent at 2 months of age in favor of the TG group. In the TG group, the averaged HF threshold slowly increases with age, roughly parallel with the WT group up to 6-8 months of age, and then stabilizes, whereas the HF thresholds in the WT group continue to deteriorate, albeit at a slightly slower rate than that seen from 2 to 8 months. This results in an increasing HF differential between the two groups. The averaged thresholds in the LF region are very close to each other between the two groups early in life (2 and 4 months). Later, the development of LF hearing loss is slower during the 4-8 months age period in TG group, resulting in a larger difference between the groups during this period. In contrast to the HF region, LFHL does not seem to stabilize in the TG group after 8 months, but rather continues to progress in a higher rate than in WT group. Therefore, LF hearing loss in the TG group catches up after 8 months of age and becomes closer to the values from the WT group by 14 months.

To further appreciate the protective effect of XIAP-Myc on hearing, the averaged ABR-threshold audiogram in WT group obtained at 6 months is compared with that from the TG groups at 14 months in FIG. 3B. Clearly, the thresholds at the three high-frequencies (16, 32 and 48 kHz) obtained at 14-month TG mice are better than those from 6 month WT mice, suggesting that the HF hearing loss was delayed by more than 8 months.

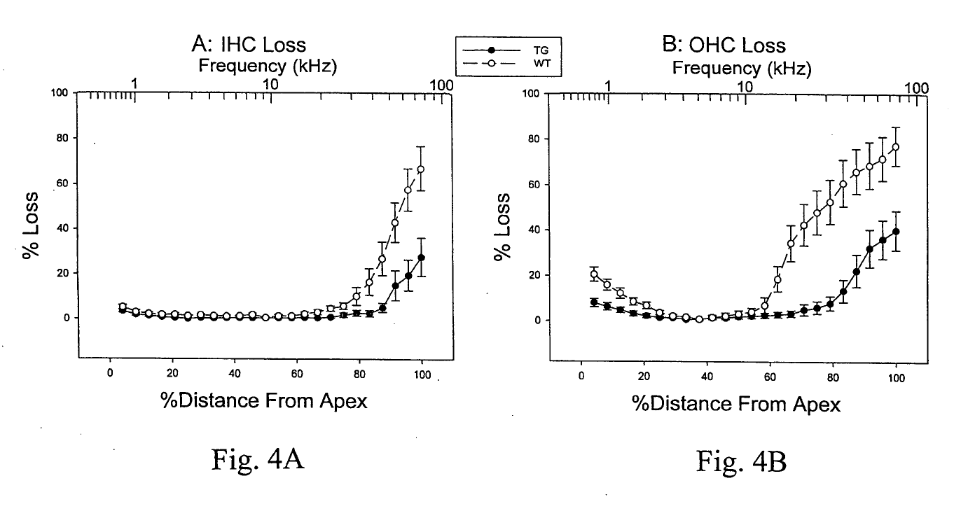

Hair cell loss was evaluated from 19 cochleae in each group. FIG. 4 compares the averaged losses of both IHCs (4A) and OHCs (4B) between the groups. Generally, the IHC loss is much less than OHC loss in the two groups and is only seen at the high-frequency end of the cochleae. In the WT group (right panel), the OHC loss is above 70% for the basal (HF) 10% end of the cochleae, spreading to the middle of the cochlea duct. In the TG group (Left panel), the OHC loss is less than 30% for the basal 10% end of the cochlea duct and the loss is restricted more to the high-frequency region. Putting the IHC and OHC losses together, the averaged hair cell loss in WT group is 665.47±417.99 cells per cochlea (mean±SD), while the value in TG group is 219.95±258.4 cells per cochlea. The difference is strongly significant (t-test, t=3.982, p<0.001).

FIG. 5 shows representative cochlea surface preparation images from two mice (two in the left panel from a TG mouse, and two in the right from a WT mouse). A smaller degree of OHC loss is seen in the TG cochlea at the very basal location (basal-1, 10% of the distance from the basal end) and the loss decreases when the as image is moved slightly towards the apex (basal-2, 10-20% of the distance from the basal end). Only scattered IHC loss is seen at the very basal end of the cochlea. In the two images taken from the WT cochlea at comparative locations, the OHC loss is much more severe. In addition, significant INC loss is also seen in these two images of the WT cochlea (FIG. 5, right panel).

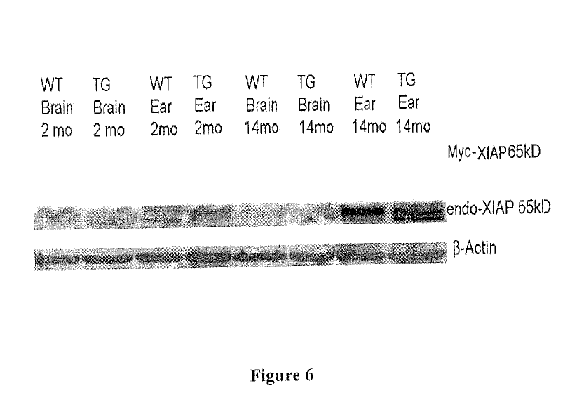

Western blot analysis was successful for 14 TG and 11 WT cochleae obtained from the mice that had been observed for 14 months and had completed their 14 month ABR. To explore further the effects of age on the expression of both endo-XIAP and XIAP-Myc, the same numbers of young mice (2 month old) in each genotype were tested. Therefore, the Western samples are divided into four groups according to ages and genotypes (young-TG, young-WT, old-TG, and old-WT). From each animal, the tissue from one cochlea and a piece of brain at the temporal lobe were used for detecting both XIAP-Myc and endo-XIAP. Relative levels of both XIAPs were calculated against the volume of β-actin to generate the volume ratio. To avoid confounding from potential technical variations across different gels, the samples were arranged, as indicated in FIG. 6, so that each gel contained 8 samples from both (2) brain tissue and ear tissue from all 4 groups (2×4). Interestingly, it was found that, while the XIAP-Myc expression seemed not change with age, the endo-XIAP appeared to increase in older ears (14 months) but not in the older brain. As shown in FIG. 7, the endo-XIAP appears to be higher in the ears than in the brain for both genotype groups. This difference is much higher in older mice. A 3-way ANOVA was performed to identify the impact of age (2 versus 14 months), genotype (WT versus TG) and tissue (brain versus cochlea) on the endo-XIAP levels. A significant effect was found for both age and tissue factors (p<0.001), but not for genotype factor (p=0.1). The null effect of genotype suggested that the transferred ub-xiap gene did not interfere with the expression of endo-xiap gene. Because of this, we analyzed the age effect by grouping the samples from both genotype groups stratified by age. Since there is no proven genotype effect on endo-XIAP level, cochleae and brain tissues from the two genotype groups are pooled for the analysis of age effect. Within the age factor, the endo-XIAP level was compared between the 25 old cochleae (from 14 month old mice after chronological ABR testing) and these from 2 month old mice of the same sample size. It appears that the endo-XIAP is much higher in the older cochlea (1.359±0.4 for 14 month cochleae versus 0.729±0.282 for 2 month-old cochleae (mean±SD), t=6.437, p<0.001). By contrast, a paired t-test failed to show a significant difference in levels of endo-XIAP in the brain for the two ages (0.525±0.178 for 2 months and 0.709±0.528 for 14 months, n=24 in each age group). Therefore, the significant age effect was largely due to the increase of endo-XIAP in the cochlea with age. Also, a significant tissue effect was indicated by the fact that the level of endo-XIAP was found to be higher in the cochlea than in the brain at both ages (t=2.921 and p=0.007 for the age of 2 months; t=4.354 and p<0.001 for the age of 14 months).

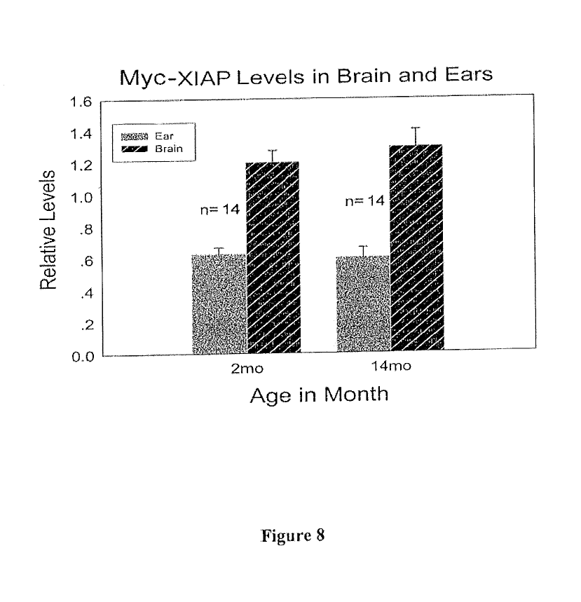

The level of XIAP-Myc appears to be independent of age, but different in the two types of tissues. FIG. 8 shows the expression of XIAP-Myc in both ears and brains for two age groups of TG mice. A 2-way ANOVA was performed against the two factors (age and tissue) that could potentially impact the level of XIAP-Myc. A significant effect of tissue was found (P<0.001). Unlike the endo-XIAP, the XIAP-Myc was found to be at a higher level in the brain than in the ear. For example, at 14 months, the XIAP-Myc is 0.601±0.24 (mean±SD) in the ear and 1.295±0.442 in the brain (t=6.621, p<0.001). However, the effect of age was not significant, suggesting that the transferred ub-xiap gene is expressed in a stable manner, which does not change with age.

II. Enzyme Facilitated Cochlear Gene Transfection with AAV

RWM becomes permeable to AAVs after digestion with the enzyme for a short period of time. Cochlear gene transfection is verified by immunostaining against GFP in the ears that are treated with the enzyme but not the untreated ear. FIG. 10 shows the transfection of a treated ear. The density of transfected cells is comparable with the ear transfected through cochleostomy. Successful transfection using this method was seen in both guinea pigs and rats.

The RWM treatment with the enzyme is functionally safe. The treatment does not cause significant hearing loss (less than 15 dB threshold shift was seen at the high end of the frequency range (32, 48 kHz), and no hearing loss at other frequencies tested. This conclusion is drawn from the results of auditory brainstem response (ABR) tests in 12 ears before and 2-4 weeks after the surgery.

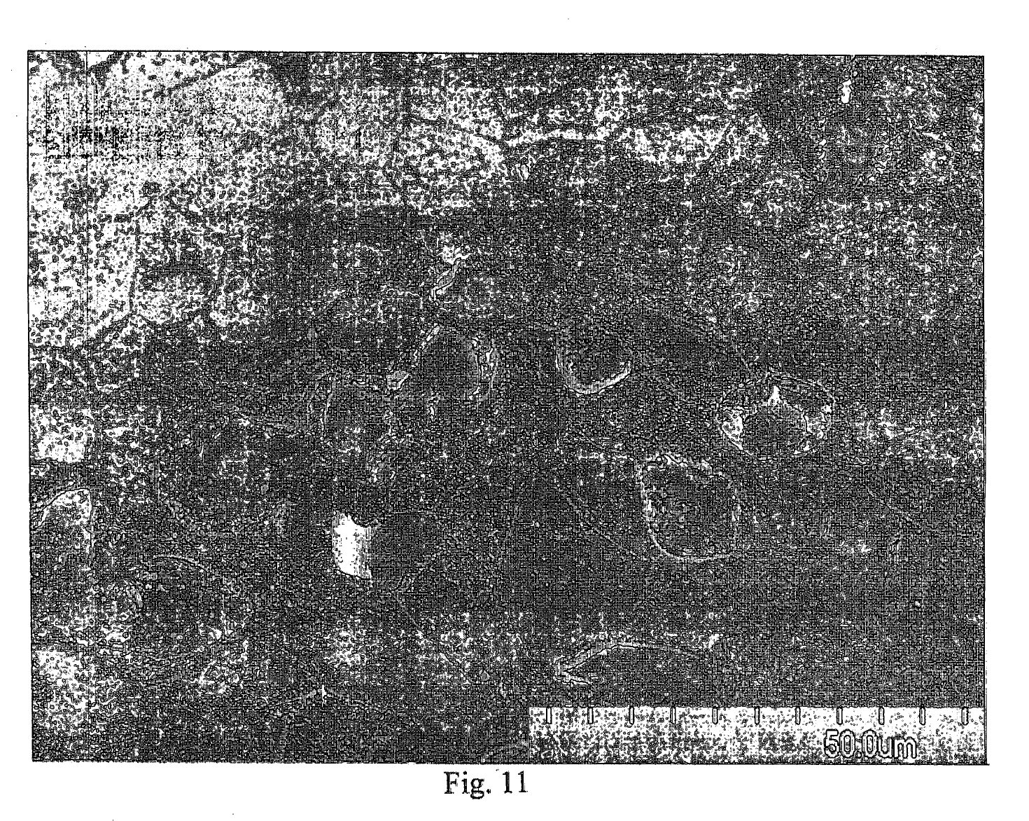

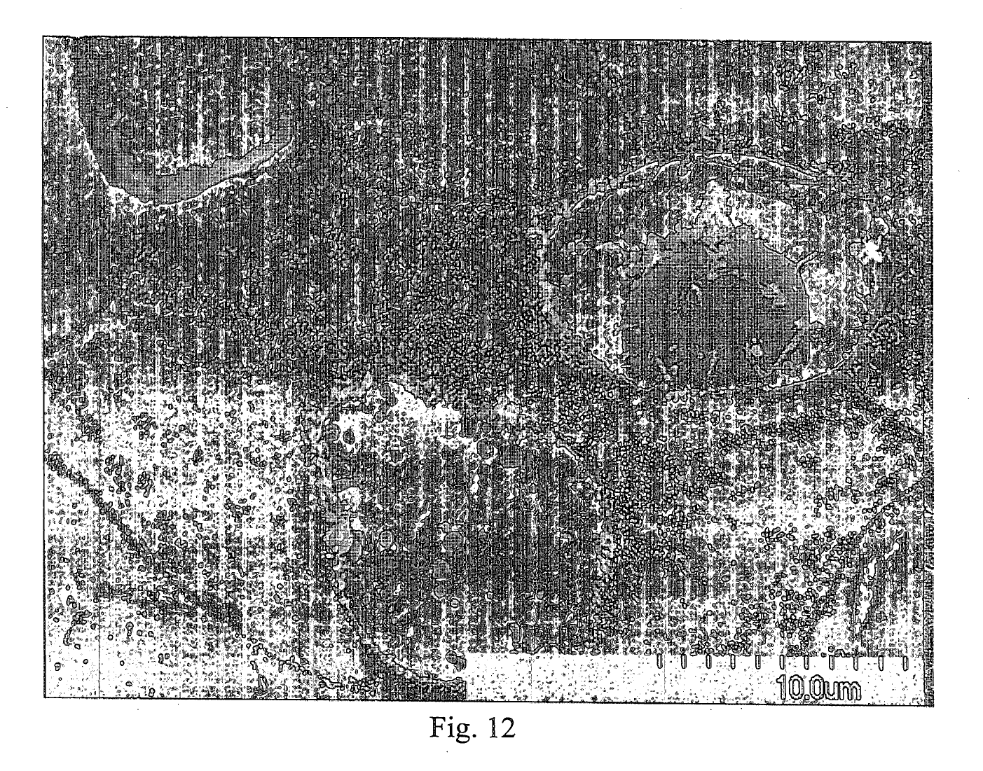

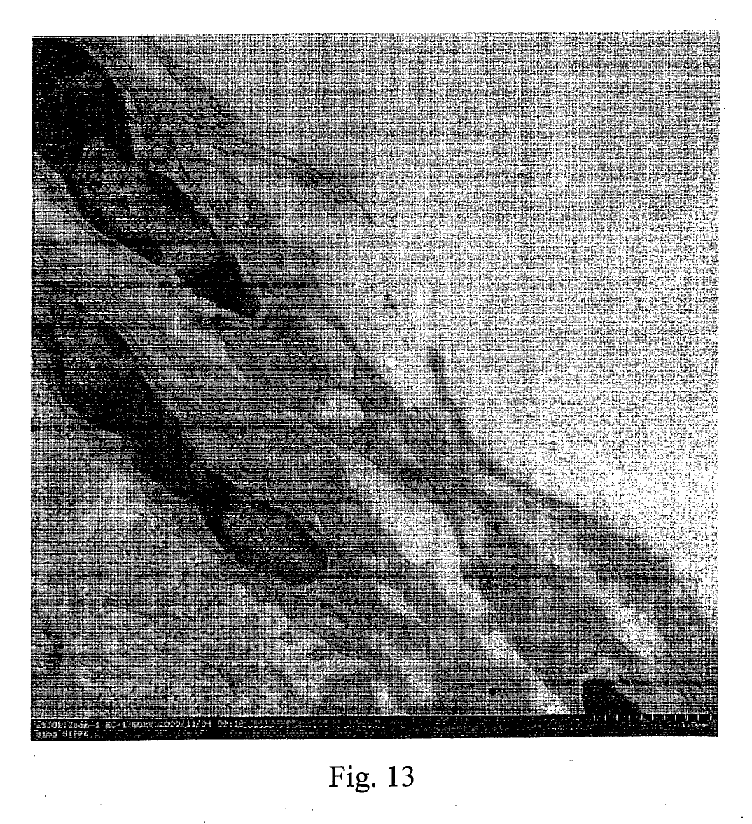

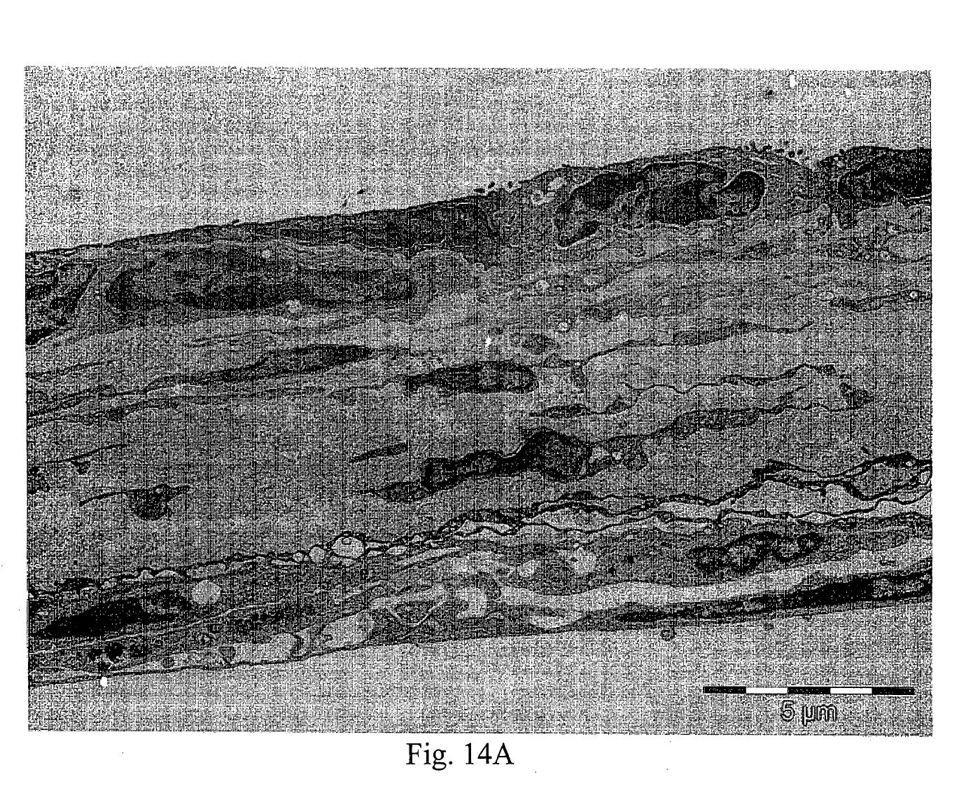

The enzyme treatment causes damage to the epithelia facing middle ear. Such damage can be seen in both SEM and TEM. FIGS. 11 and 12 show the SEM images of damaged RWM facing middle ear in two different magnitudes.

The damage to RWM by the enzyme is, however, temporary. Images of RWMs 3 weeks after the treatment show no difference from normal control. FIG. 14 shows the samples at this time point (Left: TEM, Right: SEM).

Animal models have been used previously as indicators of hearing loss in humans (see for example: Howard W. Francis et al. “The functional age of hearing loss in a mouse model of presbycusis. II. Neuroanatomical correlates.” Hearing Research 183 (2003) 29-36.; and Ohlemiller K K. “Mechanisms and genes in human strial presbycusis from animal models”. Brain Res. 2009 Jun. 24; 1277:70-83).

We found recently that the hearing sensitivity of 14 month ubXIAP mice was similar (or comparable) to that of 6 month WT mice. Hearing loss in ubXAIP mice at 6 months of age was delayed by 8 months, which is approximately ⅓ life span of this strain, which assume that a mouse lives on average for 18 months. Given that the average life expectancy for a human subject is about 80 years then we would expect to get about 25 more years of “normal” hearing using the techniques described hereinabove.

### III. Protective Effect of X-Linked Inhibitor of Apoptosis Protein Against Noise Induced Cochlear Lesion in C57 Mice

ABR Threshold Shifts after Noise Exposure

FIG. 17 shows the ABR threshold shifts 1 and 4 weeks after the exposure the noise at 25 dB SPL for 6 hours. Noise exposure resulted in threshold shifts in mid to high frequencies (4 kHz and above). There was a rapid initial partial recovery in ABR thresholds shortly after the noise exposure (data not shown), however, no further recovery was found after one week so that the two sensitivity curves for each group obtained at 1 and 4 weeks after the noise exposure were virtually identical. This shows the test-retest reliability of our ABR methodology was good. Most importantly, threshold shifts were much less (10 to 25 dB) in the TG, than the WT, group (FIG. 17). An analysis of covariance (ANCOVA) (fixed factor: genotype; covariate: frequency) identified a significant effect of genotype (F_{1,22}=260.812, p<0.001). Post-hoc tests (paired t-test with the Bonferroni correction) showed that significance was found between groups at all frequencies at both time points (1 and 4 weeks). These results indicate that TG mice engineered to express higher than normal levels of XIAP in the cochlea were protected against noise-induced hearing loss.

### Hair Cell Loss

Cytocochleograms were successfully established for 19 cochleae from TG mice group and 16 cochleae from WT controls. FIG. 18 shows the average IHC (left) and OHC (right) loss resulting from noise exposure as a function of percent distance from the cochlear apex. Unlike the wide spread of hearing loss to the low-frequency region of the cochlea, the HC losses in both IHCs and OHCs in both groups were limited to the basal end of the cochlea (within the region for sound frequency above 30 kHz). A significant amount of HC loss was seen in both groups, although HC loss was much less in TG than WT mice. This indicates that compared to WT mice, TG mice that over-express XIAP displayed reduced HCs loss after noise exposure.

FIG. 19 shows representative images of surface preparations of the cochlea in which sensory hair cells from both WT and TG mice exposed to noise are visible. These 8 low-magnification images represent 4 distinct descending levels from the apical (top four panels) to basal (bottom four panels) turns of the cochlea. These four segments from each cochlea cover more than 90% of the entire cochlea. In the representative WT mouse, HC loss (mostly the OHCs) was restricted to the extreme basal end of the cochlea that encompassed the hooked region (bottom left panel). HC loss was great in WT (bottom left panel) than TG mice (bottom right panel).

Correspondingly, the averaged total HC loss per cochlea (IHC+OHC loss) in the WT group was 501.76±247.68 (mean±S.E.M.) per ear, while the corresponding number for the TG mice was 240.37±207.80 per ear. This difference was statistically significant (t₃₃=3.645, p=<0.001). The loss of both IHCs and OHCs showed a similar trend indicating that XIAP protected both ICHs and OHCs against noise-induced death.

### Damage to SGNs and Their Fibers

The cochlear lesion induced by noise trauma was further evaluated in terms of the damage to SGN cell bodies and their dendrites to IHCs (nerve fibers). FIG. 20 shows typical images of cochlear cross-sections stained with Toluidine blue. SGN cell bodies were counted at 4 locations as indicated in FIG. 20A (1-4). The insert shows a magnified image of location 1 where SGN cell body counts were performed. The a-a line in FIG. 20A indicates the location and the orientation of the cross-section revealing Hebanular perforates in which the number of eighth nerve fibers (SGN dendrites) were counted.

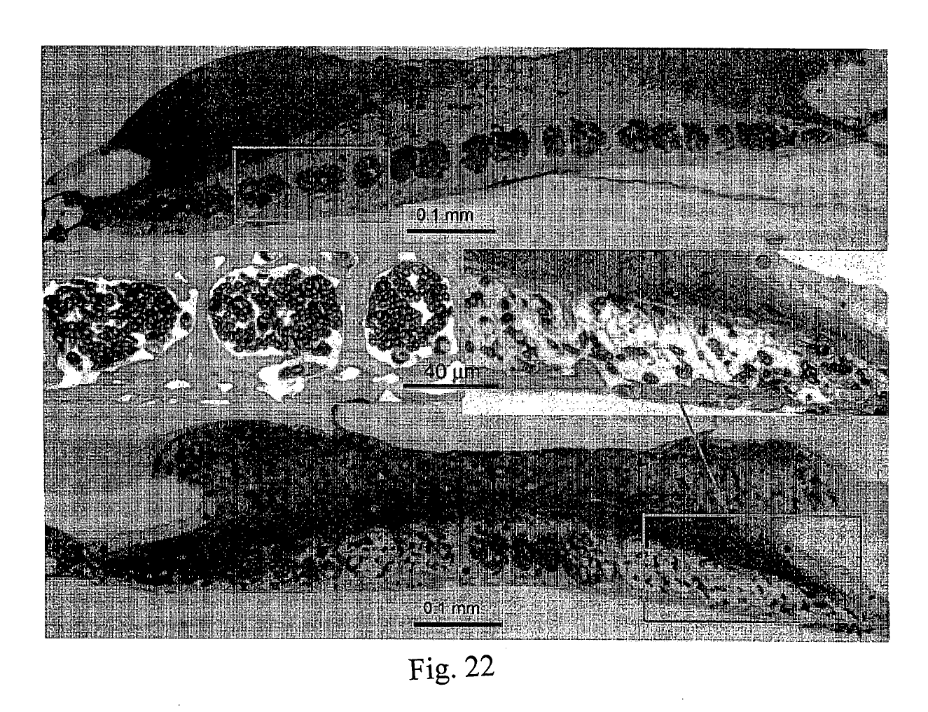

The first finding from this cross-section analysis was that noise trauma produced a reduction of the number of dendrites across a larger region of the cochlea compared to HC loss seen in the cytocochleogram. In FIG. 20B, the arrow indicates Hebanular perforates in the apical turn where there was clear dendrites loss but less threshold shift in ABR and no sensory hair cell loss. FIG. 21 demonstrates exemplary images of the Hebanular perforate from the basal turn of a cochlea of a no-noise control at low (top panel) and high magnification (bottom panel). Individual dendrites can be seen in the bottom panel of FIG. 21 that were counted to assess fiber loss. FIG. 22 shows the images taken at the levels of the basal (upper panel) and apical turns (lower panel) of a cochlea damaged by noise. In FIG. 22 (middle panels), a massive loss of dendrites was evident in both turns (left middle panel, basal turn; right middle panel, apical turn). Therefore, unlike the loss of HCs that was restricted to a small region of the cochlea (FIG. 19, lower left panel), noise exposure produced a wide spread loss of SGN dendrites in the cochlea (FIG. 22).

To quantitatively evaluate the severity of the auditory nerve lesion, the number of SGN dendrites were counted in 10 Hebanular perforates at the apical and basal turns for each cochlea. The total number of dendritic fibers counted in the 20 Hebanular perforates were 2160±48 (n=6), 1300±113 (n=10), and 1586±53 (n=10) for the no-noise control, WT and TG groups, respectively. While fiber numbers for both noise-treated groups (WT and TG) were significantly lower than the non-noise control, TG mice had significantly more dendritic fibers than WT mice (F_{2,27}=15.195, p<0.01). Hence, XIAP over-expression also protected against the loss of dendritic fibers arising from spiral ganglion neurons.

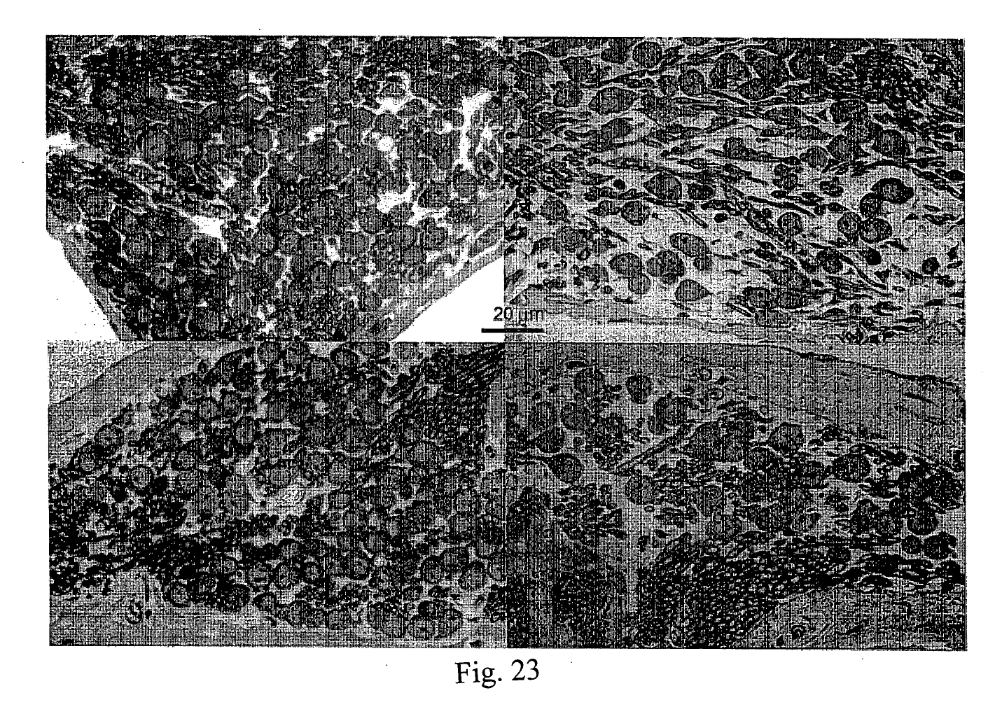

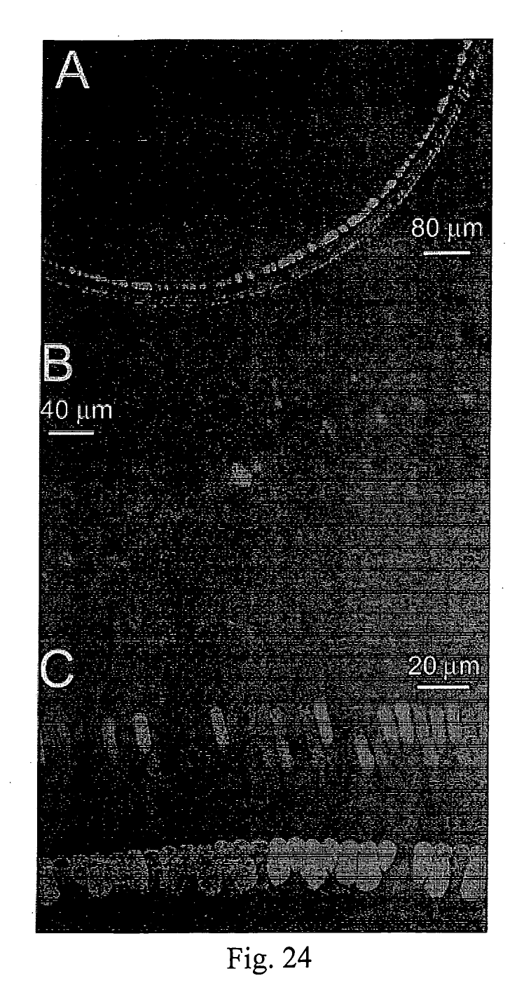

Similar to the loss of SGN dendrites there was a massive loss of SGN cell bodies following noise exposure. FIG. 23 shows images of SGN cell bodies in a cochlear cross-section through the Rosenthal canal of a no-noise control (left panels) and WT animal exposed to noise (right panels). The upper panels show images from apical turn and the lower panels correspond to the basal turn of the cochlea. In each cochlea, SGN cell bodies were counted at four levels: twice at both the basal and apical turn. At each of these levels the numbers of SGN cell bodies were averaged from two sections that were separated by over 100 μm in distance. The total of SGN cell body counts from the four levels was used as the index of SGN cell body density for each cochlea. The averaged counts for each ear were 308±11 (n=6), 222±7 (n=10) and 261±4 (n=10) for the control, WT and TG groups, respectively. Although noise exposure reduced the number of SGN cell bodies in both WT and TG groups relative to no noise controls, the reduction in SGN cell bodies for the TG group was significantly smaller (F_{2,27}=7.703, p<0.01) than that of the WT group. Consequently, elevated XIAP levels in TG mice also reduced the loss of SGN cell bodies compared to WT littermates exposed to noise.

### REFERENCES

Bohne, B. A., Harding, G. W., Lee, S. C. 2007. Death pathways in noise-damaged outer hair cells. Hear Res 223, 61-70.
Borg, E. 1987. Loss of hair cells and threshold sensitivity during prolonged noise exposure in normotensive albino rats. Hear Res 30, 119-26.
Briner, W., Willott, J. F. 1989. Ultrastructural features of neurons in the C57BL/6J mouse anteroventral cochlear nucleus: young mice versus old mice with chronic presbycusis. [Review] [29 refs]. Neurobiology of Aging 10, 295-303.
Canlon, B. 1988. The effect of acoustic trauma on the tectorial membrane, stereocilia, and hearing sensitivity: possible mechanisms underlying damage, recovery, and protection. [Review] [168 refs]. Scandinavian Audblogy. Supplementum 27, 1-45.
Chen, G. D. 2006. Prestin gene expression in the rat cochlea following intense noise exposure. Hear Res 222, 54-61.
Chen, G. D., Fechter, L. D. 2003. The relationship between noise-induced hearing loss and hair cell loss in rats. Hear Res 177, 81-90.
Chen, G. D., Liu, Y. 2005. Mechanisms of noise-induced hearing loss potentiation by hypoxia. Hear Res 200, 1-9.
Chen, G. D., Zhao, H. B. 2007. Effects of intense noise exposure on the outer hair cell plasma membrane fluidity. Hear Res 226, 14-21.
Crocker, S. J., Liston, P., Anisman, H., Lee, C. J., Smith, RD., Earl, N., Thompson, C. S., Park, D. S., Korneluk, R. G., Robertson, G. S. 2003. Attenuation of MPTP-induced neurotoxicity and behavioural impairment in NSE-XIAP transgenic mice. Neurobiol Dis 12, 150-61.
Daniel, E. 2007. Noise and hearing loss: a review. J Sch Health 77, 225-31.
Deveraux, Q. L., Reed, J. C. 1999a. IAP family proteins—suppressors of apoptosis. Genes Dev 13, 239-52.
Deveraux, Q. L., Takahashi, R., Salvesen, G. S., Reed, J. C. 1997. X-linked IAP is a direct inhibitor of cell-death proteases. Nature 388, 300-4.
Deveraux, Q. L., Stennicke, H. R., Salvesen, G. S., Reed, J. C. 1999b. Endogenous inhibitors of caspases. J Clin Immunol 19, 388-98.
Deveraux, Q. L., Roy, N., Stennicke, H. R., Van Arsdale, T., Zhou, Q., Srinivasula, S. M., Alnemri, E. S., Salvesen, G. S., Reed, J. C. 1998. IAPs block apoptotic events induced by caspase-8 and cytochrome c by direct inhibition of distinct caspases. Embo J 17, 2215-23.
Ding, D., McFadden, S., Salvi, R. 2001. Cochlear Hair Cell Densities and Inner-Ear Staining Techniques. In: Willott, J. F., (Ed.), Handbood of mouse auditory research-From behavior to molecular biology. CRC Press, New York. pp. 189-204.
Ding, D. L., McFadden, S. L., Wang, J., Hu, B. H., Salvi, R. J. 1999. Age- and strain-related differences in dehydrogenase activity and glycogen levels in CBA and C57 mouse cochleas. Audiol Neurootol 4, 55-63.
Eldadah, B. A., Faden, A. I. 2000. Caspase pathways, neuronal apoptosis, and CNS injury. J Neurotrauma 17, 811-29.
Hu, B. H., Henderson, D., Nicotera, T. M. 2002. Involvement of apoptosis in progression of cochlear lesion following exposure to intense noise. Hear Res 166, 62-71.
Hu, B. H., Henderson, D., Nicotera, T. M. 2006. Extremely rapid induction of outer hair cell apoptosis in the chinchilla cochlea following exposure to impulse noise. Hear Res 211, 16-25.
Hu, B. H., Guo, W., Wang, P. Y., Henderson, D., Jiang, S. C. 2000. Intense noise-induced apoptosis in hair cells of guinea pig cochleae. Acta Otolaryngol 120, 19-24.
Hukee, M. J., Duvall, A. J., 3rd. 1985. Cochlear vessel permeability to horseradish peroxidase in the normal and acoustically traumatized chinchilla: a reevaluation. Ann Otol Rhinol Laryngol 94, 297-303.
Ishigaki, S., Liang, Y., Yamamoto, M., Niwa, J., Ando, Y., Yoshihara, T., Takeuchi, H., Doyu, M., Sobue, G. 2002. X-Linked inhibitor of apoptosis protein is involved in mutant SOD1-mediated neuronal degeneration. J Neurochem 82, 576-84.
Kominsky, D. J., Bickel, R. J., Tyler, K. L. 2002. Reovirus-induced apoptosis requires mitochondrial release of Smac/DIABLO and involves reduction of cellular inhibitor of apoptosis protein levels. J Virol 76, 11414-24.
Kujawa, S. G., Liberman, M. C. 2006. Acceleration of age-related hearing loss by early noise exposure: evidence of a misspent youth. J Neurosci 26, 2115-23.
Kujawa, S. G., Liberman, M. C. 2008. Noise-induced primary neuronal degeneration with complete threshold recovery. In: Santi, P. A., (Ed.), Thirty-First Annual Mid-Winter Research Meeting of the Association for Research in Otolaryngology, Vol. 31. Association for Research in Otolaryngology, Phoenix, Ariz., USA. pp. 251.
Liberman, M. C. 1990. Quantitative assessment of inner ear pathology following ototoxic drugs or acoustic trauma. Toxicol Pathol 18, 138-48.
Liu, X. Z., Ouyang, X. M., Xia, X. J., Zheng, J., Pandya, A., Li, F., Du, L. L., Welch, K. O., Petit, C., Smith, R. J., Webb, B. T., Yan, D., Arnos, K. S., Corey, D., Dallos, P., Nance, W. E., Chen, Z. Y. 2003. Prestin, a cochlear motor protein, is defective in non-syndromic hearing loss. Hum Mol Genet. 12, 1155-62.
Masutani, H., Nakai, Y., Kato, A. 1995. Microvascular disorder of the stria vascularis in endolymphatic hydrops. Acta Otolaryngol Suppl 519, 74-7.
Miller, D. K. 1997. The role of the Caspase family of cysteine proteases in apoptosis. Semin Immunol 9, 35-49.
Nicholson, D. W., Thornberry, N. A. 1997. Caspases: killer proteases. Trends Biochem Sci 22, 299-306.
Nicotera, T. M., Hu, B. H., Henderson, D. 2003. The caspase pathway in noise-induced apoptosis of the chinchilla cochlea. J Assoc Res Otolaryngol 4, 466-77.
Roy, N., Deveraux, Q. L., Takahashi, R., Salvesen, G. S., Reed, J. C. 1997. The c-IAP-1 and c-IAP-2 proteins are direct inhibitors of specific caspases. Embo J 16, 6914-25.
Schoemaker, M. H., Ros, J. E., Homan, M., Trautwein, C., Liston, P., Poelstra, K., van Goor, H., Jansen, P. L., Moshage, H. 2002. Cytokine regulation of pro- and anti-apoptotic genes in rat hepatocytes: NF-kappaB-regulated inhibitor of apoptosis protein 2 (cIAP2) prevents apoptosis. J Hepatol 36, 742-50.
Simons, M., Beinroth, S., Gleichmann, M., Liston, P., Korneluk, R. G., MacKenzie, A. E., Bahr, M., Klockgether, T., Robertson, G. S., Weller, M., Schulz, J. B. 1999. Adenovirus-mediated gene transfer of inhibitors of apoptosis protein delays apoptosis in cerebellar granule neurons. J Neurochem 72, 292-301.
Smith, D. I., Lawrence, M., Hawkins, W E., Jr. 1985. Effects of noise and quinine on the vessels of the stria vascularis: an image analysis study. Am J Otolaryngol 6, 280-9.
Spongr, V., Walton, J. P., Frisina, R. D., Kazee, A. M., Flood, D. G., Salvi, R. J. 1997a. Hair cell loss and synaptic loss in inferior colliculus of C57BL/6 mice: Relationship to abnormal temporal processing. In: Syka, J., (Ed.), Auditory Signal Processing in the Central Auditory Pathway. Plenum Publishing Corp., New York.
Spongr, V. P., Flood, D. G., Frisina, R. D., Salvi, R. J. 1997b. Quantitative measure of hair cell loss in CBA and C57BL/6 mice throughout their life spans. J Acoust Soc Am 101, 3546-3553.
Suzuki, M., Yamasoba, T., Ishibashi, T., Miller, J. M., Kaga, K. 2002. Effect of noise exposure on blood-labyrinth barrier in guinea pigs. Hear Res 164, 12-8.
Suzuki, Y., Nakabayashi, Y., Takahashi, R. 2001. Ubiquitin-protein ligase activity of X-linked inhibitor of apoptosis protein promotes proteasomal degradation of caspase-3 and enhances its anti-apoptotic effect in Fas-induced cell death. Proc Natl Acad Sci USA 98, 8662-7.
Trapp, T., Korhonen, L., Besselmann, M., Martinez, R., Mercer, E. A., Lindholm, D. 2003. Transgenic mice overexpressing XIAP in neurons show better outcome after transient cerebral ischemia. Mol Cell Neurosci 23, 302-13.
Van De Water, T. R., Lallemend, F., Eshraghi, A. A., Ahsan, S., He, J., Guzman, J., Polak, M., Malgrange, B., Lefebvre, P. P., Staecker, H., Balkany, T. J. 2004. Caspases, the enemy within, and their role in oxidative stress-induced apoptosis of inner ear sensory cells. Otol Neurotol 25, 627-32.
Wang, J., Menchenton, T., Yin, S. K., Yu, Z., Bance, M., Mories, D. P., Moore, C. S., Korneluk, R. G., Robertson, G. S. 2008. Over-expression of X-linked inhibitor of apoptosis protein slows presbycusis in C57BL/6J mice. Neurobiol Aging, (online).
Wang, Y., Hirose, K., Liberman, M. C. 2002. Dynamics of noise-induced cellular injury and repair in the mouse cochlea. J Assoc Res Otolaryngol 3, 248-68.
Willott, J. F., Parham, K., Hunter, K. P. 1988. Response properties of inferior colliculus neurons in middle-aged C57BL/6J mice with presbycusis. Hear Res 37, 15-27.
Xu, D., Bureau, Y., McIntyre, D. C., Nicholson, D. W., Liston, P., Zhu, Y., Fong, W. G., Crocker, S. J., Korneluk, R. G., Robertson, G. S. 1999. Attenuation of ischemia-induced cellular and behavioral deficits by X chromosome-linked inhibitor of apoptosis protein overexpression in the rat hippocampus. J Neurosci 19, 5026-33.
Yamane, H., Nakai, Y., Konishi, K., Sakamoto, H., Matsuda, Y., Iguchi, H. 1991. Strial circulation impairment due to acoustic trauma. Acta Otolaryngol 111, 85-93.
Yang, W. P., Henderson, D., Hu, B. H., Nicotera, T. M. 2004. Quantitative analysis of apoptotic and necrotic outer hair cells after exposure to different levels of continuous noise. Hear Res 196, 69-76.
Zhao, H. B., Santos-Sacchi, J. 1999. Auditory collusion and a coupled couple of outer hair cells. Nature 399, 359-62.
Zheng, J., Richter, C. P., Cheatham, M. A. 2003a. Prestin expression in the cochlea of the reeler mouse. Neurosci Lett 347, 13-6.
Zheng, J., Shen, W., He, D. Z., Long, K. B., Madison, L. D., Dallos, P. 2000. Prestin is the motor protein of cochlear outer hair cells. Nature 405, 149-55.
Zheng, J., Long, K. B., Matsuda, K. B., Madison, L. D., Ryan, A. D., Dallos, P. D. 2003b. Genomic characterization and expression of mouse prestin, the motor protein of outer hair cells. Mamm Genome 14, 87-96.
Zimmermann, K. C., Bonzon, C., Green, D. R. 2001. The machinery of programmed cell death. Pharmacol Ther 92, 57-70.

One skilled in the art can appreciate further features and advantages of the discovery based on the above-described embodiments.

1. A method of delivering a mutated tyrosine adeno-associated viral vector or a pharmaceutically active agent to an inner ear, the method comprising: contacting the round window membrane with the mutated tyrosine adeno-associated viral vector or the pharmaceutically active agent, the permeability of the round window membrane having been enhanced to allow transport of the mutated tyrosine adeno-associated viral vector or the pharmaceutically active agent across the round window membrane so as to deliver the mutated tyrosine adeno-associated viral vector or the pharmaceutically active agent to the inner ear.

2. The method, according to claim 1, in which the mutated tyrosine adeno-associated viral expression vector expresses an ototoregenerative gene or an ototoprotective gene, the ototoregenerative gene or the ototoprotective gene being positioned in the mutated tyrosine adeno-associated viral vector for expression in an inner ear organ, or associated neural structures.

3. The method, according to claim 1, in which the permeability of the round window membrane is enhanced by contacting it with a protease or a biocompatible detergent for a time sufficient to cause the round window membrane to become partially disrupted to permit the mutated tyrosine adeno-associated viral vector or the pharmaceutically active agent to be transported thereacross.

4. The method, according to claim 3, in which the protease partially digests the membrane.

5. The method, according to claim 3, in which the protease is selected from the group consisting of: serine proteases (chymotrypsin, trypsin, elastase), threonine proteases (proteasome hydrolases), cysteine proteases (actinidain, bromelain, calpains, caspases, cathepsins, Mir1-CP, papain), aspartate proteases (cathepsin D, pepsin, chymosin), metalloproteases (collagenase, elastase, gelatinase), and glutamic acid proteases.

6. The method, according to claim 3, in which the biocompatible detergent is selected from the group consisting of: Triton X-100, Triton X-114, NP-40, Brij-35; Brij-58, Tween 20, Tween 80, Octyl glucoside, Octyl thioglucoside, SDS, CHAPS, CHAPSO, Pluronic F-127, and surfactants (Teepol, Lissapol, Alconox).

7. The method, according to claim 1, in which the permeability of the round window membrane is enhanced by disruption thereof using electroporation or electropermeabilization.

8. The method, according to claim 1, in which the permeability of the round window membrane is enhanced by contacting it with a solution containing an agent that promotes lipid peroxidation for a time sufficient to cause the round window membrane to become partially disrupted.

9. The method, according to claim 1, in which the permeability of the round window membrane is enhanced by irrigating the round window membrane with artificial perilymph for a time sufficient to cause the round window membrane to become partially disrupted.

10. The method, according to claim 1, in which the permeability of the round window membrane is enhanced by contacting the round window membrane with hyperosmolar or hyposmolar liquids or solids for a time sufficient to cause the round window membrane to become partially disrupted.

11. The method, according to claim 1, in which the permeability of the round window membrane is enhanced by passing air over it causing a mild drying effect.

12. The method, according to claim 1, in which the mutated tyrosine adeno-associated viral vector is mutated at one or more surface-exposed tyrosine residues on capsid proteins.

13. The method, according to claim 12, in which the mutated tyrosine adeno-associated viral vector is selected from the group consisting of: Tyr252 to Phe272 (Y252F), Tyr272 to Phe272 (Y272F), Tyr444 to Phe444 (Y444F), Tyr500 to Phe500 (Y500F), Tyr700 to Phe700 (Y700F), Tyr704 to Phe704), Tyr730 to Phe730 (Y730F), and Tyr 733 to Phe733 (Y733F).

14. The method, according to claim 13, in which the mutated tyrosine adeno-associated viral vector is Tyr 733 to Phe733 (Y733F).

15. The method, according to claim 2, in which the otoprotective gene is an anti-apoptotic gene, a gene encoding anti-oxidant enzymes belonging to the superoxide dismutase (SOD) family, a gene encoding neurotrophic/neuroprotective factors, a gene encoding anti-inflammatory proteins, or a gene that promotes hair cell regeneration in the vestibular system.

16. The method, according to claim 15, in which the otoprotective gene is selected from the group consisting of: Birc1a (NAIP), Birc2 (c-IAP1/HIAP-2), Birc3 (cIAP-2/HIAP-1), Birc4 (XIAP), Birc5 (survivin), Birc6 (apollon), Birc7 (livin), Birc8 (TsIAP); members of the Bcl-2 family: Bcl-2, Bcl-XL, Bcl-w, Mcl-1, Bcl-2L10, BFL-1; endogenous inhibitors of the c-Jun N-terminus kinase (JNK) known as Jun-interacting protein (JIP), JIP-1, JIP-2, JIP-3, JIP-4; SOD1, SOD2; catalase; peroxiredoxin-1, peroxiredoxin-2, glutathione preoxidase 1 (Gpx1), Gpx2, Gpx3, or Gpx4; NGF, BDNF, CNTF, GDNF, Growth/differentiation factor-15 (GDF-15), erythropoietin or vascular endothelial growth factor (VEGF); interleukin-10 (IL-10); glutathione S-transferase, Annexin-1 (ANXA1), or inhibitor of NF-κB (IκB); and ATOH-1.

17. The method, according to claim 16, in which the ototoprotective gene is full length human XIAP.

18. The method, according to claim 17, in which a ubiquitin promoter is used to drive expression of XIAP in cochlea cells.

19. A method of treating or preventing hearing loss in a subject, the method comprising: contacting the round window membrane with a mutated tyrosine adeno-associated viral vector or a pharmaceutically active agent, the permeability of the round window membrane having been enhanced to allow transport of the mutated tyrosine adeno-associated viral vector or the pharmaceutically active agent across the round window membrane so as to deliver the mutated tyrosine adeno-associated viral vector or the pharmaceutically active agent to an inner ear thereby treating or preventing the hearing loss.

20. The method, according to claim 19, in which the mutated tyrosine adeno-associated viral expression vector expresses an ototoregenerative gene or an ototoprotective gene, the ototoregenerative gene or the ototoprotective gene being positioned in the mutated tyrosine adeno-associated viral vector for expression in an inner ear organ, or associated neural structures.

21. The method, according to claim 19, in which the permeability of the round window membrane is enhanced by contacting it with a protease or a biocompatible detergent for a time sufficient to cause the round window membrane to become partially disrupted to permit the mutated tyrosine adeno-associated viral vector or the pharmaceutically active agent to be transported thereacross.

22. The method, according to claim 21, in which the protease partially digests the membrane.

23. The method, according to claim 21, in which the protease is selected from the group consisting of: serine proteases (chymotrypsin, trypsin, elastase), threonine proteases (proteasome hydrolases), cysteine proteases (actinidain, bromelain, calpains, caspases, cathepsins, Mir1-CP, papain), aspartate proteases (cathepsin D, pepsin, chymosin), metalloproteases (collagenase, elastase, gelatinase), and glutamic acid proteases.

24. The method, according to claim 21, in which the biocompatible detergent is selected from the group consisting of; Triton X-100, Triton X-114, NP-40, Brij-35; Brij-58, Tween 20, Tween 80, Octyl glucoside, Octyl thioglucoside, SDS, CHAPS, CHAPSO, Pluronic F-127, and surfactants (Teepol, Lissapol, Alconox).

25. The method, according to claim 19, in which the permeability of the round window membrane is enhanced by disruption thereof using electroporation or electropermeabilization.

26. The method, according to claim 19, in which the permeability of the round window membrane is enhanced by contacting it with a solution containing an agent that promotes lipid peroxidation for a time sufficient to cause the round window membrane to become partially disrupted.

27. The method, according to claim 19, in which the permeability of the round window membrane is enhanced by irrigating the round window membrane with artificial perilymph for a time sufficient to cause the round window membrane to become partially disrupted.

28. The method, according to claim 19, in which the permeability of the round window membrane is enhanced by contacting the round window membrane with hyperosmolar or hyposmolar liquids or solids for a time sufficient to cause the round window membrane to become partially disrupted.

29. The method, according to claim 19, in which the permeability of the round window membrane is enhanced by passing air over it causing a mild drying effect.

30. The method, according to claim 19, in which the mutated tyrosine adeno-associated viral vector is mutated at one or more surface-exposed tyrosine residues on capsid proteins.

31. The method, according to claim 30, in which the mutated tyrosine adeno-associated viral vector is selected from the group consisting of: Tyr252 to Phe272 (Y252F), Tyr272 to Phe272 (Y272F), Tyr444 to Phe444 (Y444F), Tyr500 to Phe500 (Y500F), Tyr700 to Phe700 (Y700F), Tyr704 to Phe704), Tyr730 to Phe730 (Y730F), and Tyr 733 to Phe733 (Y733F).

32. The method, according to claim 31, in which the mutated tyrosine adeno-associated viral vector is Tyr 733 to Phe733 (Y733F).

33. The method, according to claim 20, in which the otoprotective gene is an anti-apoptotic gene, a gene encoding anti-oxidant enzymes belonging to the superoxide dismutase (SOD) family, a gene encoding neurotrophic/neuroprotective factors, a gene encoding anti-inflammatory proteins, or a gene that promotes hair cell regeneration in the vestibular system.

34. The method, according to claim 33, in which the otoprotective gene is selected from the group consisting of: Birc1a (NAIP), Birc2 (c-IAP1/HIAP-2), Birc3 (cIAP-2/HIAP-1), Birc4 (XIAP), Birc5 (survivin), Birc6 (apollon), Birc7 (livin), Birc8 (TsIAP); members of the Bcl-2 family: Bcl-2, Bcl-XL, Bcl-w, Mcl-1, Bcl-2L10, BFL-1; endogenous inhibitors of the c-Jun N-terminus kinase (JNK) known as Jun-interacting protein (HP), JIP-1, JIP-2, JIP-3, JIP-4; SOD1, SOD2; catalase; peroxiredoxin-1, peroxiredoxin-2, glutathione preoxidase 1 (Gpx1), Gpx2, Gpx3, or Gpx4; NGF, BDNF, CNTF, GDNF, Growth/differentiation factor-15 (GDF-15), erythropoietin or vascular endothelial growth factor (VEGF); interleukin-10 (IL-10); glutathione S-transferase, Annexin-1 (ANXA1), or inhibitor of NF-κB (IκB); and ATOH-1.

35. The method, according to claim 34, in which the ototoprotective gene is full length human XIAP.

36. The method, according to claim 35, in which a ubiquitin promoter is used to drive expression of XIAP in cochlea cells.

37. The method, according to claim 19, in which the hearing loss is presbycusis.

38. The method, according to claim 19, in which the hearing loss is high-frequency hearing loss.

39. The method, according to claim 38, in which the high-frequency hearing loss is at 2 kHz and above.

40. The method, according to claim 19, in which the hearing loss is due to ototoxicity, noise induced hearing loss, viral infections of the inner ear, autoimmune inner ear diseases, genetic hearing losses, inner ear barotrauma; physical trauma, or surgical trauma; or inflammation.

41. The method, according to claim 40, in which the ototoxicity results from cisplatin treatment of the subject suffering from cancer.

42. The method, according to claim 2 or 20, in which the inner ear organ includes the inner ear hair cell and the outer ear hair cell.

43. The method, according to claim 42, in which the inner ear cell is a hair cell, a supporting cell, inner ear mechanical structure or a spiral ganglion neuron.

44. A method of treating hereditary hearing loss in a subject, the method comprising: contacting the round window membrane of the subject with a mutated tyrosine adeno-associated viral expression vector expressing a gene responsible for hereditary hearing loss, the permeability of the round window membrane having been enhanced to allow transport of the vector across the round window membrane, the gene responsible for hereditary hearing loss being positioned in the mutated tyrosine adeno-associated expression vector for expression in an inner ear organ, or associated neural structures, of the subject so as to treat or prevent the hearing loss.

45. The method, according to claim 44, in which the permeability of the round window membrane is enhanced by contacting it with a protease or a biocompatible detergent for a time sufficient to cause the round window membrane to become partially disrupted to permit the mutated tyrosine adeno-associated viral vector or the pharmaceutically active agent to be transported thereacross.

46. The method, according to claim 45, in which the protease partially digests the membrane.

47. The method, according to claim 46 in which the protease is selected from the group consisting of: serine proteases (chymotrypsin, trypsin, elastase), threonine proteases (proteasome hydrolases), cysteine proteases (actinidain, bromelain, calpains, caspases, cathepsins, Mir1-CP, papain), aspartate proteases (cathepsin D, pepsin, chymosin), metalloproteases (collagenase, elastase, gelatinase), and glutamic acid proteases.

48. The method, according to claim 47, in which the biocompatible detergent is selected from the group consisting of: Triton X-100, Triton X-114, NP-40, Brij-35; Brij-58, Tween 20, Tween 80, Octyl glucoside, Octyl thioglucoside, SDS, CHAPS, CHAPSO, Pluronic F-127, and surfactants (Teepol, Lissapol, Alconox).

49. The method, according to claim 44, in which the permeability of the round window membrane is enhanced by disruption thereof using electroporation or electropermeabilization.

50. The method, according to claim 44, in which the permeability of the round window membrane is enhanced by contacting it with a solution containing an agent that promotes lipid peroxidation for a time sufficient to cause the round window membrane to become partially disrupted.

51. The method, according to claim 44, in which the permeability of the round window membrane is enhanced by irrigating the round window membrane with artificial perilymph for a time sufficient to cause the round window membrane to become partially disrupted.

52. The method, according to claim 44, in which the permeability of the round window membrane is enhanced by contacting the round window membrane with hyperosmolar or hyposmolar liquids or solids for a time sufficient to cause the round window membrane to become partially disrupted.

53. The method, according to claim 44, in which the permeability of the round window membrane is enhanced by passing air over it causing a mild drying effect.

54. The method, according to claim 44, in which the mutated tyrosine adeno-associated viral vector is mutated at one or more surface-exposed tyrosine residues on capsid proteins.

55. The method, according to claim 54, in which the mutated tyrosine adeno-associated viral vector is selected from the group consisting of: Tyr252 to Phe272 (Y252F), Tyr272 to Phe272 (Y272F), Tyr444 to Phe444 (Y444F), Tyr500 to Phe500 (Y500F), Tyr700 to Phe700 (Y700F), Tyr704 to Phe704), Tyr730 to Phe730 (Y730F), and Tyr 733 to Phe733 (Y733F).

56. The method, according to claim 55, in which the mutated tyrosine adeno-associated viral vector is Tyr 733 to Phe733 (Y733F).

57. The method, according to claim 44, in which the gene is selected from the group consisting of: ACTG1, ATP2B2, CDH23, CLDN14, COCH, COL11A2, DFNA5, DFNB31, DFNB59, ESPN, EYA4, GJB2, GJB3, GJB6, KCNQ4, LHFPL5, MT-RNR1, MT-TS1, MYO1A, MYO6, MYO7A, MYO15A, OTOF, PCDH15, POU3F4, SLC26A4, STRC, TECTA, TMC1, TMIE, TMPRSS3, TRIOBP, USH1C and WFS1.

58. The method, according to claim 44, in which the hereditary hearing loss is Usher's I syndrome, Usher's II syndrome or Usher's III syndrome.

59. A method of treating or preventing impaired balance or impaired vestibular function in a subject, the method comprising: contacting the round window membrane of the subject with a mutated tyrosine adeno-associated viral expression vector, the permeability of the round window membrane having been enhanced to allow transport of the mutated tyrosine adeno-associated viral vector across the round window membrane so as to deliver the mutated tyrosine adeno-associated viral expression vector to a cell of the vestibular organ or associated neural structures, thereby treating or preventing impaired balance or impaired vestibular function.

60. The method, according to claim 59, in which the mutated tyrosine adeno-associated viral expression vector expresses an ototoregenerative gene or an ototoprotective gene, the ototoregenerative gene or the ototoprotective gene being positioned in the mutated tyrosine adeno-associated viral vector for expression in an inner ear organ, or associated neural structures.

61. The method, according to claim 59, in which the permeability of the round window membrane is enhanced by contacting it with a protease or a biocompatible detergent for a time sufficient to cause the round window membrane to become partially disrupted to permit the mutated tyrosine adeno-associated viral vector or the pharmaceutically active agent to be transported thereacross.

62. The method, according to claim 61, in which the protease partially digests the membrane.

63. The method, according to claim 62, in which the protease is selected from the group consisting of: serine proteases (chymotrypsin, trypsin, elastase), threonine proteases (proteasome hydrolases), cysteine proteases (actinidain, bromelain, calpains, caspases, cathepsins, Mir1-CP, papain), aspartate proteases (cathepsin D, pepsin, chymosin), metalloproteases (collagenase, elastase, gelatinase), and glutamic acid proteases.

64. The method, according to claim 61, in which the biocompatible detergent is selected from the group consisting of: Triton X-100, Triton X-114, NP-40, Brij-35; Brij-58, Tween 20, Tween 80, Octyl glucoside, Octyl thioglucoside, SDS, CHAPS, CHAPSO, Pluronic F-127, and surfactants (Teepol, Lissapol, Alconox).

65. The method, according to claim 59, in which the permeability of the round window membrane is enhanced by disruption thereof using electroporation or electropermeabilization.

66. The method, according to claim 59, in which the permeability of the round window membrane is enhanced by contacting it with a solution containing an agent that promotes lipid peroxidation for a time sufficient to cause the round window membrane to become partially disrupted.

67. The method, according to claim 59, in which the permeability of the round window membrane is enhanced by irrigating the round window membrane with artificial perilymph for a time sufficient to cause the round window membrane to become partially disrupted.

68. The method, according to claim 59, in which the permeability of the round window membrane is enhanced by contacting the round window membrane with hyperosmolar or hyposmolar liquids or solids for a time sufficient to cause the round window membrane to become partially disrupted.

69. The method, according to claim 59, in which the permeability of the round window membrane is enhanced by passing air over it causing a mild drying effect.

70. The method, according to claim 59, in which the mutated tyrosine adeno-associated viral vector is mutated at one or more surface-exposed tyrosine residues on capsid proteins.

71. The method, according to claim 70, in which the mutated tyrosine adeno-associated viral vector is selected from the group consisting of: Tyr252 to Phe272 (Y252F), Tyr272 to Phe272 (Y272F), Tyr444 to Phe444 (Y444F), Tyr500 to Phe500 (Y500F), Tyr700 to Phe700 (Y700F), Tyr704 to Phe704), Tyr730 to Phe730 (Y730F), and Tyr 733 to Phe733 (Y733F).

72. The method, according to claim 71, in which the mutated tyrosine adeno-associated viral vector is Tyr 733 to Phe733 (Y733F).

73. The method, according to claim 60, in which the otoprotective gene is an anti-apoptotic gene, a gene encoding anti-oxidant enzymes belonging to the superoxide dismutase (SOD) family, a gene encoding neurotrophic/neuroprotective factors, a gene encoding anti-inflammatory proteins, or a gene that promotes hair cell regeneration in the vestibular system.

74. The method, according to claim 73, in which the otoprotective gene is selected from the group consisting of: Birc1a (NAIP), Birc2 (c-IAP1/HIAP-2), Birc3 (cIAP-2/HIAP-1), Birc4 (XIAP), Birc5 (survivin), Birc6 (apollon), Birc7 (livin), Birc8 (TsIAP); members of the Bcl-2 family: Bcl-2, Bcl-XL, Bcl-w, Mcl-1, Bcl-2L10, BFL-1; endogenous inhibitors of the c-Jun N-terminus kinase (JNK) known as Jun-interacting protein (JIP), JIP-1, JIP-2, JIP-3, JIP-4; SOD1, SOD2; catalase; peroxiredoxin-1, peroxiredoxin-2, glutathione preoxidase 1 (Gpx1), Gpx2, Gpx3, or Gpx4; NGF, BDNF, CNTF, GDNF, Growth/differentiation factor-15 (GDF-15), erythropoietin or vascular endothelial growth factor (VEGF); interleukin-10 (IL-10); glutathione S-transferase, Annexin-1 (ANXA1), or inhibitor of NF-κB (IκB); and ATOH-1.

75. The method, according to claim 74, in which the ototoprotective gene is full length human XIAP.

76. The method, according to claim 59 in which the impaired balance is in a subject who is aging.

77. The method, according to claim 59 in which the impaired vestibular function is result of vestibular organ degeneration.

78. The method, according to claim 77, in which the vestibular organ regeneration is due to ototoxicity, viral infections of the inner ear, autoimmune inner ear diseases, genetic vestibular losses, inner ear barotraumas; or physical trauma, or surgical trauma.

79. The method, according to any one of the above claims, in which the subject is human.
